SEQUENCE LISTING (56 sequences)

SEQ ID NO: 1 - protein, 178 aa, human
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Artificial Sequence
  lipocalin mutein
QDSTSDLIPAPPLSKVPLQQNFQDNQFHGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - protein, 272 aa, human
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 15 aa, Artificial Sequence
  linker
GGGGSGGGGSGGGGS

SEQ ID NO: 5 - protein, 18 aa, Artificial Sequence
  linker
PSTPPTNSSSTPPTPSPS

SEQ ID NO: 6 - protein, 14 aa, Artificial Sequence
  linker
GGSGNSSGSGGSPV

SEQ ID NO: 7 - protein, 20 aa, Artificial Sequence
  linker
ASPAAPAPASPAAPAPSAPA

SEQ ID NO: 8 - protein, 66 aa, Artificial Sequence
  linker
AGSGGSGGSGGSPVPSTPPTPSPSTPPTPSPSGGSGNSSGSGGSPVPSTPPTPSPSTPPTPSPSAS

SEQ ID NO: 9 - protein, 32 aa, Artificial Sequence
  linker
PSTPPTPSPSTPPTPSPSGGSGNSSGSGGSPV

SEQ ID NO: 10 - protein, 74 aa, Artificial Sequence
  linker
AGSGGSGGSGGSPVPSTPPTNSSSTPPTPSPSPVPSTPPTNSSSTPPTPSPSPVPSTPPTNSSSTPPTPSPSAS

SEQ ID NO: 11 - protein, 40 aa, Artificial Sequence
  linker
ASPAAPAPASPAAPAPSAPAASPAAPAPASPAAPAPSAPA

SEQ ID NO: 12 - protein, 10 aa, Artificial Sequence
  linker
VDDIEGRMDE

SEQ ID NO: 13 - protein, 11 aa, Artificial Sequence
  linker
ENLYFQGRMDE

SEQ ID NO: 14 - protein, 10 aa, Artificial Sequence
  linker
GGGGSGGGGS

SEQ ID NO: 15 - protein, 327 aa, Artificial Sequence
  antibody heavy chain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 214 aa, Artificial Sequence
  antibody light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 228 aa, Artificial Sequence
  antibody Fc region
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 228 aa, Artificial Sequence
  antibody Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 228 aa, Artificial Sequence
  antibody Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 20 - protein, 228 aa, Artificial Sequence
  antibody Fc region
ESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYNVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 22 - protein, 178 aa, Artificial Sequence
  lipocalin mutein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 23 - protein, 178 aa, Artificial Sequence
  lipocalin mutein
QDSTSDLIPAPPLSKVPLQQNFQDNQFHGKWYVVGQAGNIRLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 24 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 25 - protein, 175 aa, Artificial Sequence
  lipocalin mutein
QDSTSDLIPAPPLSKVPLQQNFQDNQFHGKWYVVGQAGNIKLREDSKMMATIYELKEDKSYDVTGVSFDDKKCTYAIMTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 26 - protein, 178 aa, Artificial Sequence
  lipocalin mutein
QDSTSDLIPAPPLSKVPLQQNFQDNQFHGKWYVVGQAGNIKLREDKDPVKMMATIYELKEDKSYDVTGVTFDDKKCRYDISTFVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 27 - protein, 178 aa, Artificial Sequence
  lipocalin mutein
QDSTSDLIPAPPLSKVPLQQNFQDNQFHGKWYVVGQAGNIRLREDKDPHKMMATIYELKEDKSYDVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 28 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYDVTGVTFDDKKCTYAISTLVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 29 - protein, 178 aa, Artificial Sequence
  lipocalin mutein
QDSTSDLIPAPPLSKVPLQQNFQDNQFHGKWYVVGQAGNIRLREDKDPSKMMATIYELKEDKSYDVTAVTFDDKKCNYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 30 - protein, 178 aa, Artificial Sequence
  lipocalin mutein
QDSTSDLIPAPPLSKVPLQQNFQDNQFHGKWYVVGMAGNNLLREDKDPHKMSATIYELKEDKSYNVTDVMFLDKKCQYIIWTFVPGSQPGEFTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 31 - protein, 178 aa, Artificial Sequence
  lipocalin mutein
QDSTSDLIPAPPLSKVPLQQNFQDNQFQGKWYVVGMAGNNLLREDKDPHKMSATIYELKEDKSYNVTDVMFLDKKCQYIIWTFVPGSQPGELTLGFIRSDLGHTSYLVRVVSTNYNQHAMVFFKSVIQNREWFGITLYGRTKELTSELKENFIRFSKSLGLPENHIVFPVPIDQCIDG

SEQ ID NO: 32 - protein, 178 aa, Artificial Sequence
  lipocalin mutein
QDSTSDLIPAPPLSKVPLQQNFQDYQFQGKWYVVGMAGNNLLREDKDPHKMGATIYELKEDKSYNVTDVMLLDKKCQYIIQTFVPGSQPGESTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 33 - protein, 178 aa, Artificial Sequence
  lipocalin mutein
QDSTSDLIPAPPLSKVPLQQNFQDNQFQGKWYVVGMAGNNLLREDKDPHKMGATIYELKEDKSYNVTDVMFLDKKCQHIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 34 - protein, 178 aa, Artificial Sequence
  lipocalin mutein
QDSTSDLIPAPPLSKVPLQQNFQDDQFQGKWYVVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 35 - protein, 178 aa, Artificial Sequence
  lipocalin mutein
QDSTSDLIPAPPLSKVPLQQNFQDNQFQGKWYIVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 36 - protein, 178 aa, Artificial Sequence
  lipocalin mutein
QDSTSDLIPAPPLSKVPLQRNFQDNQFQGKWYVVGMAGNNLLRVDKDPHKMGATIYELKEDKSYNVTDVMFLDKKCQYIIWTFVPGSQPGELTLGFIKSDPGHTSYLVRVVSTNYNQHAMVYFKSVIQNREWFGITLYGRTKELTSELKENFIRFSKSLGLPENHIVFPVPIDQCIDG

SEQ ID NO: 37 - protein, 178 aa, Artificial Sequence
  lipocalin mutein
QDSTSDLIPAPPLSKVPLQQNFQDNQFQGKWYVVGMAGNNLLREDKDPHKMSATIYELKEDKSYNVTDVMFLDKKCQYINWPFVPGSQPGEFTLGFIKSDLGPTSYLVRVVSTNYNQHAMVFFKSVIQNREWFGITLYGRTKELTSELKENFIRFSKSLGLPENHIVFPVPIDQCIDG

SEQ ID NO: 38 - protein, 178 aa, Artificial Sequence
  lipocalin mutein
QDSTSDLIPAPPLSKVPLQQNFQDNQFQGKWYVVGMAGNNLLREDKDPHKMGATIYELNEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 39 - protein, 178 aa, Artificial Sequence
  lipocalin mutein
QDSTSDLIPAPPLSKVPLQQNFQDNQFQGKWYVVGMAGNNLLRDDKDPHKMSATIYELKEDKSYNVTDVMLLDKKCHYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 40 - protein, 10 aa, Artificial Sequence
  antibody CDR
GFNIKDTYIH

SEQ ID NO: 41 - protein, 18 aa, Artificial Sequence
  antibody CDR
RIYPTNGYTRYADSVKGR

SEQ ID NO: 42 - protein, 11 aa, Artificial Sequence
  antibody CDR
WGGDGFYAMDY

SEQ ID NO: 43 - protein, 12 aa, Artificial Sequence
  antibody CDR
RASQDVNTAVAW

SEQ ID NO: 44 - protein, 7 aa, Artificial Sequence
  antibody CDR
SASFLYS

SEQ ID NO: 45 - protein, 9 aa, Artificial Sequence
  antibody CDR
QQHYTTPPT

SEQ ID NO: 46 - protein, 120 aa, Artificial Sequence [sequence omitted]

SEQ ID NO: 47 - protein, 107 aa, Artificial Sequence [sequence omitted]

SEQ ID NO: 48 - protein, 450 aa, Artificial Sequence
  antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 447 aa, Artificial Sequence
  antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 640 aa, Artificial Sequence
  fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 52 - protein, 407 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 53 - protein, 640 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 407 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 449 aa, Artificial Sequence
  antibody heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 219 aa, Artificial Sequence
  antibody light chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0166764 A1
(43) Pub. Date: May 23, 2024

(54) HER2/4-1BB BISPECIFIC FUSION PROTEINS FOR THE TREATMENT OF CANCER

(71) Applicant: Pieris Pharmaceuticals GmbH, Hallbergmoos (DE)

(72) Inventors: Markus ZETTL, Hallbergmoos (DE); Aizea MORALES KASTRESANA, Hallbergmoos (DE); Cornelia WURZENBERGER, Hallbergmoos (DE); Shane OLWILL, Hallbergmoos (DE); Kayti AVIANO, Hallbergmoos (DE)

(21) Appl. No.: 18/552,059

(22) PCT Filed: Mar. 23, 2022
(86) PCT No.: PCT/EP2022/057606

§ 371 Date: Sep. 22, 2023

Related U.S. Application Data
(60) Provisional application No. 63/164,797, filed on Mar. 23, 2021.

(51) Int. Cl. C07K 16/32; A61K 38/00; A61K 45/06; A61P 35/00; C07K 14/47

(52) U.S. Cl. CPC C07K 16/32; A61K 45/06; A61P 35/00; C07K 14/47; A61K 38/00

(57) ABSTRACT

The disclosure provides methods and compositions for treating tumors, particularly HER2-expressing tumors. The method comprises administering a therapeutically effective amount of a HER2/4-1BB bispecific fusion protein. The HER2/4-1BB bispecific fusion protein may be administered at a first dose and, subsequently, at a second dose, wherein the first dose exceeds the second dose.

### I. BACKGROUND

4-1BB, also known as CD137, is a co-stimulatory immune receptor and a member of the tumor necrosis factor receptor (TNFR) super-family. 4-1BB plays an important role in the regulation of immune responses and thus is a target for cancer immunotherapy. 4-1BB ligand (4-1BBL) is the only known natural ligand of 4-1BB and is constitutively expressed on several types of antigen presenting cells (APCs), such as activated B cells, monocytes, and splenic dendritic cells. 4-1BB can also be induced on T lymphocytes.

HER2, or HER2/neu, is a member of the human epidermal growth factor receptor family. Amplification or overexpression of this oncogene has been shown to play an important role in the development and progression of a variety of tumors, including certain aggressive types of breast cancer. HER2 has been shown to be highly differentially expressed on certain tumor cells, with much higher cell-surface density on those cells compared to healthy tissue.

Lipocalins are proteinaceous molecules that can be engineered to bind ligands. Muteins of various lipocalins (lipocalin muteins) are a rapidly expanding class of therapeutics and can be constructed through highly sophisticated artificial engineering to exhibit a high affinity and specificity against a target that is different than a natural ligand of wild-type lipocalins (see, e.g., WO 99/16873, WO 00/75308, WO 03/029463, WO 03/029471 and WO 05/19256).

### II. SUMMARY

PRS-343 (cinrebafusp alfa) is a HER2/4-1BB bispecific antibody-lipocalin mutein fusion protein, developed as the first 4-1BB-based bispecific therapeutic. The present disclosure is based on clinical studies of PRS-343 in patients with HER2-positive (HER2+) advanced or metastatic solid tumors.

The present disclosure provides, among other things, compositions comprising a HER2/4-1BB bispecific antibody-lipocalin mutein fusion protein and methods of administering said compositions. Methods and compositions described herein have been shown to be safe and efficacious in treating HER2-expressing tumors, including HER2+ tumors and tumors characterized by a low expression of HER2.

In some embodiments, the methods include administering a HER2/4-1BB bispecific fusion protein at a first dose and, subsequently, at a second dose, wherein the first dose exceeds the second dose.

### III. DEFINITIONS

The following list defines terms, phrases, and abbreviations used throughout the instant specification. All terms listed and defined herein are intended to encompass all grammatical forms.

As used herein, unless otherwise specified, “4-1BB” means human 4-1BB (hu4-1BB). Human 4-1BB means a full-length protein defined by UniProt Q07011, a fragment thereof, or a variant thereof. 4-1BB is also known as CD137, tumor necrosis factor receptor superfamily member 9 (TNFRSF9) and induced by lymphocyte activation (ILA). In some particular embodiments, 4-1BB of non-human species, e.g., cynomolgus 4-1BB and mouse 4-1BB, is used.

As used herein, unless otherwise specified, “HER2” means human HER2 (huHER2). Human HER2 means a full-length protein defined by UniProt P04626, a fragment thereof, or a variant thereof. HER2 is also known as human epidermal growth factor receptor 2, HER2/neu, receptor tyrosine-protein kinase erbB-2, cluster of differentiation 340 (CD340), proto-oncogene Neu, ERBB2 (human), Erbb2 (rodent), c-neu, or p185. Human HER2 is encoded by the ERBB2 gene. In some particular embodiments, HER2 of non-human species, e.g., cynomolgus HER2 and mouse HER2, is used.

The term “anti-”, when used to describe a molecule in association with a protein target of interest (e.g., 4-1BB or HER2), means the molecule is capable of binding the protein target and/or modulating one or more biological functions of the protein target. For example, an “anti-4-1BB” molecule as described herein, is capable of binding 4-1BB and/or modulating one or more biological functions of 4-1BB. “Biological function” of a protein target refers to the ability of the protein target to carry out its biological mission(s), e.g., binding to its binding partner(s) and mediating signaling pathway(s).

As used herein, “T cell activation” refers to a process leading to proliferation and/or differentiation of T cells. The activation of T cells may lead to the initiation and/or perpetuation of immune responses. As used herein, T cell activation may be used to assess the health of subjects with disease or disorders associated with dysregulated immune responses, such as cancer, autoimmune disease, and inflammatory disease. T cell proliferation refers to the expansion of a T cell population. “T cell proliferation” and “T cell expansion” are used interchangeably herein.

The terms “enhance T cell activity”, “activate T cells”, and “stimulate T cell response”, are used interchangeably herein and refer to inducing, causing, or stimulating T cells to have sustained or amplified biological functions, or renew or reactivate exhausted or inactive T cells. Exemplary signs of enhanced T cell activity include, but are not limited to: increased secretion of interleukin-2 (IL-2) from T cells, increased secretion of Interferon-gamma (IFN-γ) from T cells, increased T cell proliferation, and/or increased antigen responsiveness (e.g., viral, pathogen, and tumor clearance). Methods of measuring such enhancement are known to the skilled in the art.

“Cancer” and “cancerous” refers to the physiological condition in mammals that is typically characterized by unregulated cell growth. A “tumor” may comprise one or more cancerous cells. A “lesion” is a localized change in a tissue or an organ. Tumors are types of lesions. “Target lesions” are lesions that have been specifically measured. “Non-target lesions” are lesions whose presences have been noted, but whose measurements have not been taken. The terms “cancer”, “tumor”, and “lesion” are used interchangeably herein.

As used herein, the term “HER2-expressing tumor” is meant to refer to a tumor with detectable expression of HER2, e.g., detectable by a quantitative assay, such as an mRNA-based qRT-PCR assay. In some embodiments, the term “HER2-expressing tumor” refers to a HER2-positive (HER2+) tumor or to a tumor characterized by a low expression of HER2.

The term “HER2-positive (HER2+) tumor”, as used herein, is not particularly limited as long as it is recognized as such tumor by a person skilled in the art. In some embodiments, the term “HER2-positive (HER2+) tumor” is meant to refer to a tumor which is classified as a HER2+ tumor by immunohistochemistry (IHC) and/or (fluorescent) in situ hybridization ((F)ISH) analysis, e.g., according to the 2018 ASCO/CAP guidelines for HER2 testing in breast cancer (Wolff et al., 2018) or the 2016 CAP/ASCP/ASCO guidelines for HER2 testing in gastric or gastroesophageal adenocarcinoma (Bartley et al., 2016). In some particular embodiments, a HER2+ tumor is characterized by a HER2 status of IHC3+, IHC2+/(F)ISH+ or (F)ISH+, preferably IHC3+ or IHC2+/(F)ISH+. In some embodiments, a HER2+ tumor is characterized by HER2 gene amplification, e.g., as determined by (F)ISH or next generation sequencing (NGS) analysis.

As used herein, the term “tumor characterized by a low expression of HER2” (also referred to herein as “HER2 low tumor”) is not particularly limited as long as it is recognized as such tumor by a person skilled in the art. In some embodiments, a “tumor characterized by a low expression of HER2” refers to a tumor which exhibits expression of HER2, albeit at a level which does not warrant its classification as a HER2+ tumor by IHC and (F)ISH. In some embodiments, a HER2 low tumor is a tumor which exhibits expression of HER2 at a level which is detectable by a quantitative assay, such as an mRNA-based qRT-PCR assay, but which is not classified as a HER2+ tumor by IHC and/or (F)ISH, e.g., according to the 2018 ASCO/CAP guidelines for HER2 testing in breast cancer (Wolff et al., 2018) or the 2016 CAP/ASCP/ASCO guidelines for HER2 testing in gastric or gastroesophageal adenocarcinoma (Bartley et al., 2016). In some particular embodiments, a HER2 low tumor is characterized by a HER2 status of IHC1+ or IHC2+/(F)ISH− (i.e., IHC2+ without HER2 gene amplification). However, for the avoidance of doubt, HER2 low tumors may also include tumors that are, for example, characterized by a HER2 status of IHC0 (and (F)ISH−), but that still exhibit expression of HER2, e.g., as determined in a quantitative assay, such as an mRNA-based qRT-PCR assay. In some embodiments, a HER2 low tumor does not exhibit HER2 gene amplification, e.g., as determined by (F)ISH or next generation sequencing (NGS) analysis.

The term “metastatic” refers to a state of cancer where the cancer cells break away from where they first formed and form new tumors (metastatic tumors) in other parts of the body. An “advanced” cancer may be locally advanced or metastatic. Locally advanced cancer refers to cancer that has grown outside the site or organ of origin but has not yet spread to distant parts of the body.

“Tumor microenvironment (TME)” refers to the environment around a tumor, composed of non-cancer cells and their stroma. The tumor stroma comprises a compilation of cells, including fibroblasts/myofibroblasts, glial, epithelial, fat, immune, vascular, smooth muscle, and immune cells, blood vessels, signaling molecules, and the extracellular matrix (ECM), and serves a structural or connective role. In this context, “full tumor tissue” consists of tumor cells and tumor stroma.

As used herein, an “anti-tumor agent” or “anti-tumor drug” may act on a tumor, particularly a malignant tumor, and preferably has an anti-tumor effect or anti-tumor activity. The “anti-tumor effect” or “anti-tumor activity” refers to actions of an anti-tumor agent on a tumor, particularly a malignant tumor, including stimulation of tumor-specific immune responses, reduction in target lesion, reduction in tumor size, suppression of the growth of tumor cells, suppression of metastasis, complete remission, partial remission, stabilization of disease, extension of the term before recurrence, extension of survival time of patients, or improvement of quality of life of patients.

As used herein, “treat” or “treatment” refers to clinical intervention designed to alter the natural course of the subject being treated during the course of a physiological condition or disorder or clinical pathology. A treatment may be a therapeutic treatment and/or a prophylactic or preventative measure, wherein the object is to prevent or slow down (lessen) an undesired physiological change or disorder, such as the growth, development or spread of a hyperproliferative condition, such as cancer. Desired effects of treatment include, but are not limited to, decreasing the rate of disease progression, ameliorating or palliating the disease state, alleviating symptoms, stabilizing or not worsening the disease state, and remission of improved prognosis, whether detectable or undetectable. Desired effects of treatment also include prolonging survival as compared to expected survival if not receiving treatment. A subject in need of treatment includes a subject already with the condition or disorder or prone to have the condition or disorder or a subject in which the condition or disorder is to be prevented.

A treatment given to a subject with tumor may lead to tumor response as described in Response Evaluation Criteria in Solid Tumors (RECIST) guideline (version 1.1) (Eisenhauer et al., 2009). For example, a treatment given to a subject with tumor may lead to complete response, partial response, stable disease, or progressive disease. “Complete response (CR)” refers to the disappearance of all target lesions. “Partial response (PR)” refers to at least a 30% decrease in the sum of diameters of target lesions, taking as reference the baseline sum diameters. “Progressive disease (PD)” refers to At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. “Stable disease (SD)” refers to neither sufficient shrinkage to qualify for PR nor sufficient increase to qualify for PD, taking as reference the smallest sum diameters while on study. “Duration of response (DoR)” may be calculated as the time from the date of first documented response (CR or PR) to the date of documented progression or death after achieving response.

An “effective amount” of a drug or therapeutic agent is an amount sufficient to effect beneficial or desired effects of a treatment. For example, an effective amount an anti-tumor agent may be one that is sufficient to enhance T cell activation to a desired level. In some embodiments, the effectiveness of a drug or therapeutic agent can be determined by suitable methods known in the art. For example, the effectiveness of an anti-tumor agent may be determined by Response Evaluation Criteria in Solid Tumors (RECIST). An effective amount can be administered in one or more individual administrations or doses. An effective amount can be administered alone with one agent or in combination with one or more additional agents.

As used herein, “antibody” includes whole antibodies or any antigen binding fragment (i.e., “antigen-binding domain”) or single chain thereof. A whole antibody refers to a glycoprotein comprising at least two heavy chains (HCs) and two light chains (LCs) inter-connected by disulfide bonds. Each heavy chain is comprised of a heavy chain variable domain (V_{H} or HCVR) and a heavy chain constant region (C_{H}). The heavy chain constant region is comprised of three domains, C_{H1}, C_{H2} and C_{H3}. Each light chain is comprised of a light chain variable domain (V_{L} or LCVR) and a light chain constant region (C_{L}). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen (for example, PD-L1). The constant regions of the antibodies may optionally mediate the binding of the immunoglobulin to host tissues or factors, including various cells of the immune system (e.g., effector cells) and the first component (C1q) of the classical complement system.

As used herein, “antigen-binding domain” or “antigen-binding fragment” of an antibody refers to one or more fragments of an antibody that retain the ability to specifically bind to an antigen (e.g., HER2). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H1} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between C_{H1} and C_{H2} domains; (iv) an Fd fragment consisting of the V_{H} and C_{H1} domains; (v) a single-chain Fv fragment consisting of the V_{H} and V_{L} domains of a single arm of an antibody, (vi) a dAb fragment (Ward et al., 1989) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., 1993); (ix) a “domain antibody fragment” containing only the V_{H} or V_{L}, where in some instances two or more V_{H} regions are covalently joined.

Antibodies may be polyclonal or monoclonal; xenogeneic, allogeneic, or syngeneic; or modified forms thereof (e.g., humanized, chimeric, or multispecific). Antibodies may also be fully human.

The term “effector functions” as used herein with respect to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the term “lipocalin” refers to a monomeric protein of approximately 18-20 kDa in weight, having a cylindrical β-pleated sheet supersecondary structural region comprising a plurality of β-strands (preferably eight β-strands designated A to H) connected pair-wise by a plurality of (preferably four) loops at one end to thereby comprise a ligand-binding pocket and define the entrance to the ligand-binding pocket. Preferably, the loops comprising the ligand-binding pocket used in the present invention are loops connecting the open ends of β-strands A and B, C and D, E and F, and G and H, and are designated loops AB, CD, EF, and GH. It is well-established that the diversity of the said loops in the otherwise rigid lipocalin scaffold gives rise to a variety of different binding modes among the lipocalin family members, each capable of accommodating targets of different sizes, shape, and chemical character (reviewed, e.g. in Skerra, 2000, Flower et al., 2000, Flower, 1996). It is understood that the lipocalin family of proteins has naturally evolved to bind a wide spectrum of ligands, sharing unusually low levels of overall sequence conservation (often with sequence identities of less than 20%) yet retaining a highly conserved overall folding pattern. The correspondence between positions in various lipocalins is also well-known to one of skill in the art (see, e.g., U.S. Pat. No. 7,250,297). Proteins falling in the definition of “lipocalin” as used herein include, but are not limited to, human lipocalins including tear lipocalin (Tlc, Lcn1), Lipocalin-2 (Lcn2) or neutrophil gelatinase-associated lipocalin (NGAL), apolipoprotein D (ApoD), apolipoprotein M, α₁-acid glycoprotein 1, α₁-acid glycoprotein 2, α₁-microglobulin, complement component 8γ, retinol-binding protein (RBP), the epididymal retinoic acid-binding protein, glycodelin, odorant-binding protein IIa, odorant-binding protein IIb, lipocalin-15 (Lcn15), and prostaglandin D synthase.

As used herein, “Lipocalin-2” or “neutrophil gelatinase-associated lipocalin” refers to human Lipocalin-2 (hLcn2) or human neutrophil gelatinase-associated lipocalin (hNGAL) and further refers to the mature human Lipocalin-2 or mature human neutrophil gelatinase-associated lipocalin. The term “mature” when used to characterize a protein means a protein essentially free from the signal peptide. A “mature hNGAL” of the instant disclosure refers to the mature form of human neutrophil gelatinase-associated lipocalin, which is free from the signal peptide. Mature hNGAL is described by residues 21-198 of the sequence deposited with the SWISS-PROT Data Bank under Accession Number P80188, the amino acid sequence of which is indicated in SEQ ID NO: 1.

As used herein, a “native sequence” refers to a protein or a polypeptide having a sequence that occurs in nature or having a wild-type sequence, regardless of its mode of preparation. Such native sequence protein or polypeptide can be isolated from nature or can be produced by other means, such as by recombinant or synthetic methods.

The “native sequence lipocalin” refers to a lipocalin having the same amino acid sequence as the corresponding polypeptide derived from nature. Thus, a native sequence lipocalin can have the amino acid sequence of the respective naturally-occurring (wild-type) lipocalin from any organism, in particular, a mammal. The term “native sequence”, when used in the context of a lipocalin specifically encompasses naturally-occurring truncated or secreted forms of the lipocalin, naturally-occurring variant forms such as alternatively spliced forms and naturally-occurring allelic variants of the lipocalin. The terms “native sequence lipocalin” and “wild-type lipocalin” are used interchangeably herein.

As used herein, a “mutein,” a “mutated” entity (whether protein or nucleic acid), or “mutant” refers to the exchange, deletion, or insertion of one or more amino acids or nucleotides, compared to the naturally-occurring (wild-type) protein or nucleic acid. Said term also includes fragments of a mutein as described herein. The present disclosure explicitly encompasses lipocalin muteins, as described herein, having a cylindrical β-pleated sheet supersecondary structural region comprising eight β-strands connected pair-wise by four loops at one end to thereby comprise a ligand-binding pocket and define the entrance of the ligand-binding pocket, wherein at least one amino acid of each of at least three of said four loops has been mutated as compared to the native sequence lipocalin. Lipocalin muteins of the present disclosure preferably have the function of binding 4-1BB as described herein.

As used herein, the term “fragment,” in connection with the lipocalin muteins of the disclosure, refers to proteins or polypeptides derived from full-length mature hNGAL or lipocalin muteins that are N-terminally and/or C-terminally truncated, i.e., lacking at least one of the N-terminal and/or C-terminal amino acids. Such fragments may include at least 10 or more, such as 20 or 30 or more, consecutive amino acids of the primary sequence of mature hNGAL or the lipocalin mutein it is derived from and are usually detectable in an immunoassay of mature hNGAL. Such a fragment may lack up to 2, up to 3, up to 4, up to 5, up to 10, up to 15, up to 20, up to 25, or up to 30 (including all numbers in between) of the N-terminal and/or C-terminal amino acids. It is understood that the fragment is preferably a functional fragment of mature hNGAL or the lipocalin mutein from which it is derived, which means that it preferably retains the binding specificity, preferably to 4-1BB, of mature hNGAL or the lipocalin mutein it is derived from. As an illustrative example, such a functional fragment may comprise at least amino acids at positions 13-157, 15-150, 18-141, 20-134, 25-134, or 28-134 corresponding to the linear polypeptide sequence of mature hNGAL.

A “fragment” with respect to 4-1BB or HER2 refers to N-terminally and/or C-terminally truncated 4-1BB or HER2 or protein domains of 4-1BB or HER2. Fragments of 4-1BB or HER2 as described herein retain the capability of the full-length 4-1BB or HER2 to be recognized and/or bound by a lipocalin mutein, an antibody, and/or a fusion protein of the disclosure.

As used herein, “bispecific” refers to a molecule is able to specifically bind to at least two distinct targets. Typically, a bispecific molecule comprises two target-binding sites, each of which is specific for a different target. In some embodiments, the bispecific molecule is capable of simultaneously binding two targets.

As used interchangeably herein, the terms “conjugate,” “conjugation,” “fuse,” “fusion,” or “linked” refer to the joining together of two or more subunits, through all forms of covalent or non-covalent linkage, by means including, but not limited to, genetic fusion, chemical conjugation, coupling through a linker or a cross-linking agent, and non-covalent association.

The term “fusion polypeptide” or “fusion protein” as used herein refers to a polypeptide or protein comprising two or more subunits. In some embodiments, a fusion protein as described herein comprises two or more subunits, at least one of these subunits being capable of specifically binding to 4-1BB, and a further subunit capable of specifically binding to HER2. Within the fusion protein, these subunits may be linked by covalent or non-covalent linkage. Preferably, the fusion protein is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. However, the subunits of a fusion protein of the present disclosure may also be linked through chemical conjugation. The subunits forming the fusion protein are typically linked to each other as follows: C-terminus of one subunit to N-terminus of another subunit, or C-terminus of one subunit to C-terminus of another subunit, or N-terminus of one subunit to N-terminus of another subunit, or N-terminus of one subunit to C-terminus of another subunit. The subunits of the fusion protein can be linked in any order and may include more than one of any of the constituent subunits. If one or more of the subunits is part of a protein (complex) that consists of more than one polypeptide chain, the term “fusion protein” may also refer to the protein comprising the fused sequences and all other polypeptide chain(s) of the protein (complex). As an illustrative example, where a full-length immunoglobulin is fused to a lipocalin mutein via a heavy or light chain of the immunoglobulin, the term “fusion protein” may refer to the single polypeptide chain comprising the lipocalin mutein and the heavy or light chain of the immunoglobulin. The term “fusion protein” may also refer to the entire immunoglobulin (both light and heavy chains) and the lipocalin mutein fused to one or both of its heavy and/or light chains.

As used herein, the term “subunit” of a fusion protein disclosed herein refers to a single protein or a separate polypeptide chain, which may form a stable folded structure by itself and define a unique function of providing a binding motif towards a target. In some embodiments, a preferred subunit of the disclosure is a lipocalin mutein. In some other embodiments, a preferred subunit of the disclosure is a full-length immunoglobulin or an antigen-binding domain thereof.

A “linker” that may be comprised by a fusion protein of the present disclosure joins together two or more subunits of a fusion protein as described herein. The linkage can be covalent or non-covalent. A preferred covalent linkage is via a peptide bond, such as a peptide bond between amino acids. A preferred linker is a peptide linker. Accordingly, in a preferred embodiment, said linker comprises one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. Other preferred linkers include chemical linkers.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100. A skilled artisan will recognize available computer programs, for example BLAST (Altschul et al., 1997), BLAST2 (Altschul et al., 1990), FASTA (Pearson and Lipman, 1988), GAP (Needleman and Wunsch, 1970), Smith-Waterman (Smith and Waterman, 1981), and Wisconsin GCG Package, for determining sequence identity using standard parameters. The percentage of sequence identity can, for example, be determined herein using the program BLASTP, version 2.2.5, Nov. 16, 2002 (Altschul et al., 1997), calculating the percentage of numbers of “positives” (homologous amino acids) from the total number of amino acids selected for the alignment.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity.

A “sample” is defined as a biological sample taken from any subject. Biological samples include, but are not limited to, blood, serum, urine, feces, semen, or tissue, including tumor tissue.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, to name only a few illustrative examples. Preferably, the “mammal” used herein is human.

As used herein the term “about” or “approximately” means within 20%, preferably within 15%, preferably within 10%, and more preferably within 5% of a given value or range. It also includes the concrete number, i.e. “about 20” includes the number of 20. The term “at least about” as used herein includes the concrete number, i.e., “at least about 20” includes 20.

As used herein, the term “and/or” includes the meaning of “and,” “or,” and “all or any other combination of the elements connected by said term.”

As used herein, the singular forms “a,” “an,” and “the” include plural referents unless the context clearly dictates otherwise.

### IV. DESCRIPTIONS OF FIGURES

FIG. 1: provides the results of an in vitro T cell immunogenicity assessment of the HER2/4-1BB bispecific fusion proteins (SEQ ID NOs: 50 and 51, SEQ ID NOs: 50 and 53, SEQ ID NOs: 52 and 49, and SEQ ID NOs: 54 and 49), reference antibody SEQ ID NOs: 50 and 48, and positive control keyhole limpet hemocyanine (KLH). The assay was performed using a PBMC-based format as described in Example 1, with 32 donors and human leukocyte antigen (HLA) allotypes reflective of the distribution in a global population. FIG. 1A presents the stimulation index (proliferation in the presence vs. absence of test article). The average responses are indicated as bars. The threshold that defines a responding donor (stimulation index >2) is indicated as a dotted line. FIG. 2B shows the number of responders for each test article.

FIG. 2: shows the cell-based activity of PRS-343 to co-stimulate T cell activation in a target-dependent manner. Purified human T cells (FIG. 2A) or a 4-1BB overexpressing-Jurkat NF-κB reporter cell line (FIG. 2B) were co-cultured with HER2-expressing tumor cell lines (NCI-N87 (HER2 high), MKN45 (HER2 low), and HepG2 (HER2 null)), or without tumor cells, in the presence of PRS-343. In the presence of HER2-positive cell lines, a dose-dependent induction of IL-2 or 4-1BB clustering and downstream signaling in Jurkat NF-κB reporter cells was observed with PRS-343. All data depicted here are representative illustrations of experiments carried out with minimum two different donors. Statistical analysis: *, P<0.05; **, P<0.01; and ***, P<0.001, using one-way ANOVA with Dunnet multiple comparison test.

FIG. 3: depicts the accelerated titration design of the Phase 1, open-label, dose escalation study of PRS-343 (FIG. 3A) and the overall study design (FIG. 3B).

FIG. 4: depicts the overall study design.

FIG. 5: shows the geometric mean PRS-343 serum concentration-time profiles after a single dose (the first dose, administered Cycle 1 Day 1 administration), ranging from 0.015 mg/kg to 8 mg/kg. The 8 mg/kg plot includes patients in both Cohort 11 (8 mg/kg, Q3W) and 11B (8 mg/kg, Q2W).

FIG. 6: presents the drug exposure/pharmacodynamics relationship for Cohorts 1 to 11B (dose levels ranging from 0.0005 mg/kg Q3W to 8 mg/kg Q2W).

FIG. 7: shows the CD8+ T cell expansion in full tumor tissue (FIG. 7A), tumor stroma (FIG. 7B), and tumor cells (FIG. 7C) in patients receiving PRS-343. The increase of CD8+ T cells is more pronounced for patients in Cohort 9 of the study and onwards (dose levels 2.5 mg/kg) as compared to low dose Cohorts 1-8.

FIG. 8: shows the CD8+ T cell expansion in full tumor tissue (FIG. 8A), tumor stroma (FIG. 8B), and tumor cells (FIG. 8C) in the responding patient 107-012. The increase of CD8+ T cells are more pronounced in tumor cells than in full tumor tissue or tumor stroma.

FIG. 9: shows the CD8+ T cell expansion in full tumor tissue (FIG. 9A), tumor stroma (FIG. 9B), and tumor cells (FIG. 9C) in the responding patient 108-002. The increase of CD8+ T cells are more pronounced in tumor cells than in full tumor tissue or tumor stroma.

FIG. 10: shows the CD8+Ki67+ T cell expansion in full tumor tissue (FIG. 10A), tumor stroma (FIG. 10B), and tumor cells (FIG. 10C) in the responding patient 108-002. The increase of CD8+Ki67+ T cells is only observed in tumor cells.

FIG. 11: shows the average time on treatment with PRS-343 is increased in Cohort 11B (8 mg/kg, Q2W) compared to Cohorts 9 to 11 (2.5 mg/kg, 5 mg/kg, and 8 mg/kg, respectively, Q3W).

FIG. 12: depicts the best response in target lesions for Cohorts 1 to 11B (FIG. 12A) and Cohorts 9 to 11B (FIG. 12B).

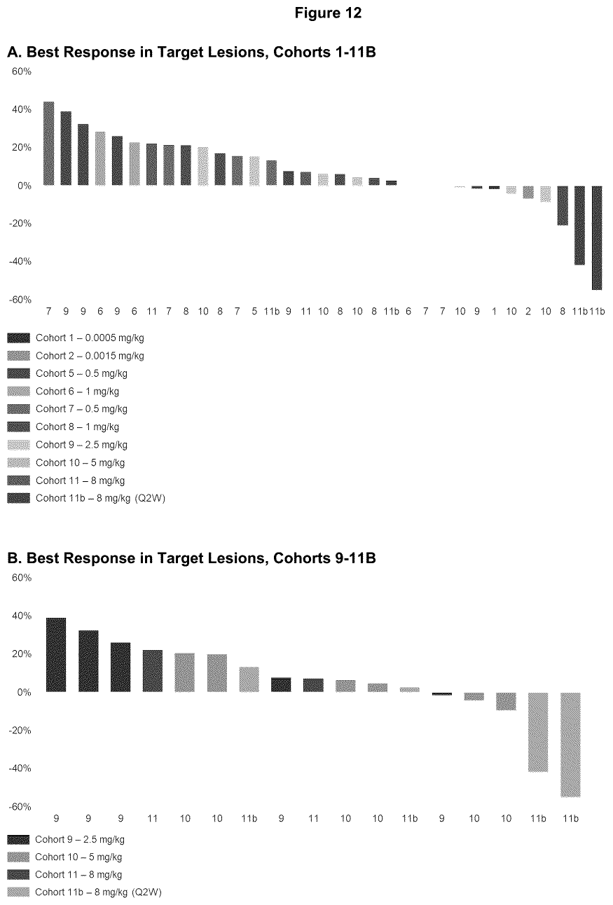

FIG. 13: provides an overview over the design of HER2/4-1BB bispecific fusion proteins as described herein. Representative HER2/4-1BB bispecific fusion proteins were made based on an antibody specific for HER2 (e.g., an antibody shown in SEQ ID NOs: 50 and 48) and a lipocalin muteins specific for 4-1BB (e.g., a lipocalin mutein shown in SEQ ID NO: 22). One or more anti-4-1BB lipocalin muteins were genetically fused, via a peptide linker, at the N-terminus or the C-terminus, to an anti-HER2 antibody at the C-terminus of the antibody heavy chain domain (HC) (FIG. 13D), the N-terminus of the HC (FIG. 13A), the C-terminus of the antibody light chain (LC) (FIG. 13C), and/or the N-terminus of the LC (FIG. 13B), resulting in the fusion proteins such as SEQ ID NOs: 50 and 51, SEQ ID NOs: 50 and 53, SEQ ID NOs: 52 and 49, and SEQ ID NOs: 54 and 49. An engineered IgG4 backbone with the mutations S228P, F234A, and L235A was used for the anti-HER2 antibody as included in the fusion proteins.

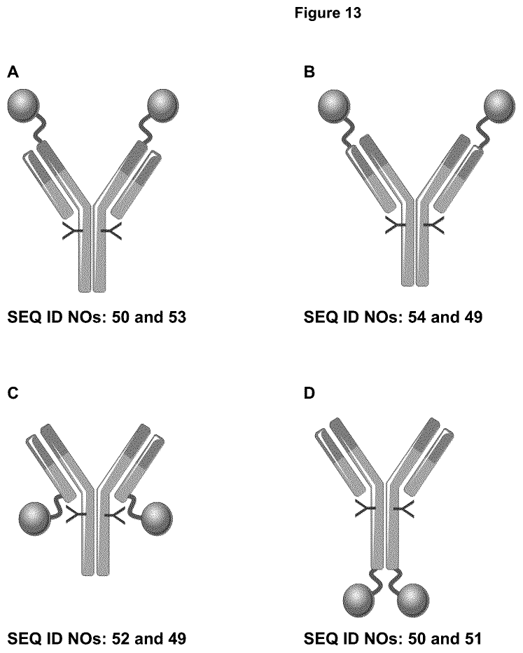

FIG. 14: shows the geometric mean PRS-343 serum concentration-time profiles after a single dose (the first dose, cycle 1, day 1), ranging from 0.015 mg/kg to 18 mg/kg. The 8 mg/kg plot includes patients in both Cohort 11 (8 mg/kg, Q3W) and 11B (8 mg/kg, Q2W). The 12 mg/kg plot includes patients in Cohort 12B (12 mg/kg, Q2W), the 18 mg/kg includes patients in Cohort 13B (18 mg/kg, Q2W).

FIG. 15: shows CD8+ T cell expansion in full tumor tissue (FIG. 15A) and serum levels of soluble 4-1BB (s4-1BB) (FIG. 15 B) of patients in non-active dose Cohorts 1-8 vs. patients in the active dose Cohorts 9-13B. Patients treated with an active dose of PRS-343 showed increased CD8+ T cells in the tumor tissue and circulating s4-1 BB, demonstrating 4-1 BB arm activity of PRS-343.

FIG. 16: shows the course of treatment for patients in Cohorts 11B, 11C, 12B, 13B and Obi+11B, including the clinical status (where applicable).

FIG. 17: depicts the best response in target lesions for Cohorts 9, 10, 11, 11B, 11C, 12B, 13B and Obi+11B.

FIG. 18: shows CD8+ T cell expansion (x-fold induction) vs. % growth/shrinkage of target lesion in active dose cohorts. Patients with SD≥C6, PR and CR exhibited an at least 2.3-fold increase of CD8+ T cells.

FIG. 19: shows CT scans of a target lesion (lung; see dark circle) in the responding patient 103-021 at baseline, C2 post-treatment and C6 post-treatment. The patient showed a complete response (CR).

FIG. 20: shows post-treatment CD8+ T cell expansion in full tumor tissue (FIG. 20A) and an increase of circulating s4-1BB in the serum (FIG. 20B) of the CR patient 103-021, demonstrating 4-1BB arm activity of PRS-343.

FIG. 21: shows CT scans of target lesions (see dark circles) in the responding patient 107-012 at baseline and C4 post-treatment. The patient showed a partial response (PR).

FIG. 22: shows post-treatment CD8+ T cell and CD8+Ki67+ T cell expansion in full tumor tissue (FIG. 22A) and an increase of circulating s4-1BB in the serum (FIG. 22B) of the PR patient 107-012, demonstrating 4-1BB arm activity of PRS-343.

FIG. 23: shows a repeated increase of circulating s4-1BB in the serum of the PR patient 103-012 over the course of multiple treatment cycles.

FIG. 24: shows pre-treatment absolute numbers of CD8+ T cells in full tumor tissue of active cohort patients split up in “PD & SD<C6” and “CR, PR & SD>C6” patients (FIG. 24A) and a plot of % PD-L1+ cells of total immune cells (IC score) vs. pre-treatment absolute numbers of CD8+ T cells for individual responding patients of active dose cohorts (FIG. 24B). PRS-343 drives clinical benefit in PD-L1 low/negative patients and patients with low CD8+ T cell counts prior to therapy.

FIG. 25: shows the s4-1BB profiles of two clinical responders, breast cancer patient 103-016 (FIG. 25A; stable disease at cycles 2 and 4) and colorectal cancer patient 103-019 (FIG. 25B; stable disease at cycles 2, 4 and 6). Biopsy analysis revealed that the tumors of these patients were characterized by a low expression of HER2, as indicated by a HER2 status of IHC2+/FISH− and IHC0 or 1+/FISH−, respectively.

FIG. 26: shows PRS-343 serum concentration-time profiles after a single dose (the first dose, cycle 1, day 1) in cohort 11B (8 mg/kg, Q2W; FIG. 26A) and cohort 13B (18 mg/kg, Q2W; FIG. 26B); grey lines: individual patients; black line: geometric mean of respective cohort.

FIG. 27: shows the dose dependency of CD8+ T cell expansion in full tumor tissue (measured on day 15 of cycle 1; FIG. 27A) and serum levels of s4-1BB (measured over the course of cycle 1; FIG. 27B) upon treatment with PRS-343 across all tested dose cohorts (8 mg/kg data include data of patients treated Q1W, Q2W or Q3W); grey line: connects group averages; black lines: median; Mann-Whitney U test was used for statistical analysis.

FIG. 28: shows the geometric mean PRS-343 serum concentration-time profiles after a single dose (the first dose, cycle 1, day 1; FIG. 28A) and repeated dosing (profile after 5^{th} dose on day 1 of cycle 3; FIG. 28B) of patients dosed with 8 mg/kg Q2W or 18 mg/kg Q2W, or of a patient dosed with a loading dose of 18 mg/kg Q2W in cycle 1, followed by a lower dose of 8 mg/kg Q2W in subsequent cycles. PRS-343 serum concentrations were measured using an electrochemiluminescence (ECL) assay. Briefly, free PRS-343 in serum samples was captured on a microtiter plate coated with human CD137 (4-1BB) protein (SinoBiological). Bound PRS-343 was then detected via an anti-trastuzumab antibody (Clona 5A4, Abnova) followed by SULFO-TAG-labeled anti-mouse antibody (Meso Scale Diagnostics). Pharmacokinetic parameters were derived by non-compartmental analyses and are based on nominal time points.

### V. DETAILED DESCRIPTION OF THE DISCLOSURE

4-1BB is a co-stimulatory immune checkpoint and member of the tumor necrosis factor receptor (TNFR) family. It is primarily expressed on activated CD4+ and CD8+ T cells, activated B cells, and natural killer (NK) cells, and plays an important role in the regulation of the immune response. The clustering of 4-1BB leads to activation of the receptor and downstream signaling (Yao et al., 2013, Snell et al., 2011). In a T cell pre-stimulated by the T cell receptor (TCR) binding to a cognate major histocompatibility complex (MHC) target, co-stimulation via 4-1BB leads to enhanced activation, survival, and proliferation, as well as to the production of pro-inflammatory cytokines and an improved capacity to kill (Dawicki and Watts, 2004, Lee et al., 2002).

In line with the mode of 4-1BB activation, which requires receptor clustering, a monospecific 4-1BB-targeting agent, such as an anti-4-1BB antibody, may not be efficient by itself to cluster 4-1BB and lead to efficient activation. Additionally, a monospecific 4-1BB-targeting agent may lead to non-localized 4-1BB clustering and activation, because the expression of 4-1BB is not limited to tumor infiltrating lymphocytes (Makkouk et al., 2016, Alizadeh et al., 2011). Recent work on TNFR family members also illustrates the mechanisms of anti-TNFR antibodies, whereby the antibodies interact via their Fc regions with Fc-gamma receptors, engage activating Fc-gamma receptor-expressing immune cells, and facilitate the subsequent anti-tumor activity (Bulliard et al., 2014, Bulliard et al., 2013), suggesting an anti-4-1BB antibody may trigger 4-1BB clustering depending on the abundance of Fc-gamma receptor-positive cells but not restricted to a tumor microenvironment.

Accordingly, the efficacy and toxicity are in fact major concerns of an anti-4-1BB monotherapy. Ongoing clinical trials of two agonist antibodies, urelumab and utomilumab, present significant challenges. Urelumab has substantial toxicity at doses above 1 mg/kg and is demonstrated safe at only 0.1 mg/kg (every 3 weeks). Clinical efficacy results with low-dose urelumab monotherapy, however, were largely ineffective and there has been limited clinical activity of urelumab at the tolerated dose (Massarelli et al., 2016, Segal et al., 2017). Utomilumab is tolerated at a higher dose (up to 10 mg/kg every 4 weeks) but is a less potent 4-1BB agonist relative to urelumab and has potential efficacy challenges (Tolcher et al., 2017, Chester et al., 2018, Segal et al., 2018).

Therefore, there is an unmet need for 4-1BB-targeting therapeutics that are both effective and safe. An ideal 4-1BB-targeting agent should lead to clustering of 4-1BB, and do so in a tumor localized fashion on tumor-infiltrating lymphocytes to minimize safety risk. Such a 4-1BB-targeting agent should be able to engage tumor specific CD8+ T cells, so that efficacy may be achieved at tolerant dose levels. As described herein, to obtain such a 4-1BB-targeting agent, bispecific agents may be designed to target 4-1BB on one end and a differentially expressed tumor target on the other end.

In this respect, HER2 is a clinically-validated target across a broad spectrum of tumor types. Amplification of the HER2 gene and overexpression of its product have been shown to play an important role in the development and progression of various types of cancer including breast, bladder, gastric, gastroesophageal, colorectal, and biliary tract cancer. Anti-HER2 therapeutics such as trastuzumab, a monoclonal antibody to HER2, accrue significant clinical benefit in patients with early stage or metastatic HER2-positive (HER2+) breast cancer. However, many patients with metastatic disease do not respond to therapy or develop refractory disease, and some patients suffer disease recurrence. For example, trastuzumab monotherapy in the metastatic setting results in response rates of 11-26% (clinical benefit rate: 48%), implying that many HER2+ tumors will not respond to monotherapy (Vogel et al., 2002). Meanwhile, no biomarker beyond HER2 has demonstrated clinical utility for patient selection for anti-HER2 therapy in HER2-positive breast cancer, and no biomarker of response or resistance have yet been clinically validated.

Therefore, there remains the need for better targeted therapy for patient with HER2-positive cancer. There also remains the need to identify biomarkers associated with favorable patient populations and beneficial clinical outcomes.

The present disclosure provides new therapies including 4-1BB targeting agents. As described herein, a 4-1BB targeting agent comprises a fusion protein, having at least two binding domains, where one binding domain comprises a lipocalin mutein engineered to specifically bind 4-1BB and a second binding domain which comprises an antibody or antigen binding domain thereof specific for HER2.

As described above, lipocalin muteins have a cylindrical β-pleated sheet supersecondary structural region comprising eight β-strands connected pair-wise by four loops at one end. These loops comprise a ligand-binding pocket and define the entrance of the ligand-binding pocket. The loop regions forming the binding pocket of a lipocalin have been compared to the 6 complementarity-determining regions (CDRs) of an antibody. Similar to antibodies, the loop regions confer target binding specificity and mutating this region can alter binding properties of the lipocalin. Resulting muteins are sometimes referred to as “Anticalins”, and Anticalin technology has been described in the literature (see Skerra (2000 Biochim Biophys Acta (1482) 337-350, WO 03/029462A1; Pieris Proteolab AG, and Schonfeld et al. (2009) Proc. Natl. Acad. Sci. USA 106, 8198-8203).

The present disclosure provides lipocalin muteins, as part of a bispecific fusion proteins, comprising particular mutations within the four loop regions of the ligand-binding pocket, resulting in muteins with binding specificity towards a non-natural target (e.g., 4-1BB).

The inventors have shown that lipocalins can be engineered by introducing particular sets of mutations within the loop regions in order to confer binding to 4-1BB (a non-natural target) (see WO 2016/177762, which is herein incorporated by reference in its entirety). Additionally, said lipocalin muteins have been included in a fusion format, where the fusions have been shown to be capable of simultaneous binding of 4-1BB and HER2 (see WO 2016/177802, which is herein incorporated by reference in its entirety). The present disclosure provides the use of said 4-1BB/HER2 fusion proteins in pharmaceutical compositions in order to treat HER2-expressing tumors, e.g., HER2+ tumors or tumors characterized by a low expression of HER2, in human patients, and particular methods of treatment to achieve clinical results.

In some embodiments, HER2/4-1BB bispecific fusion proteins as provided herein are envisioned to bring HER2-expressing tumor cells and 4-1BB-expressing T cells to proximity and promote 4-1BB clustering and signaling, to inhibit HER2 signaling, deliver a co-stimulatory signal to tumor antigen-specific T cells providing localized immune activation, and facilitate tumor cell killing and tumor destruction.

As described herein, PRS-343 (also referred to as Cinrebafusp alfa) is a HER2/4-1BB bispecific fusion protein, promoting 4-1BB clustering by bridging 4-1BB-positive T cells with HER2-expressing tumor cells, and thereby providing a potent co-stimulatory signal to tumor antigen-specific T cells. PRS-343 is designed to localize CD137 activation in the tumor in a HER2-dependent manner. The amino acid sequence of PRS-343 is shown in SEQ ID NOs: 50 and 51.

The present disclosure provides results of a first-in-human Phase 1 study of PRS-343 conducted in patients with (presumed) HER2+ advanced or metastatic solid tumors to assess the safety and efficacy of PRS-343. Following administration of PRS-343, the pharmacokinetic (PK) profile, pharmacodynamic (PD) effects, and PK/PD correlations were determined.

Based on the results of the study as presented herein, the present disclosure provides a method for treating a HER2-expressing tumor in a subject, comprising administering a therapeutically effective amount of a HER2/4-1BB bispecific fusion protein, such as one comprising the amino acid sequences set forth in SEQ ID NOs: 50 and 51. The present disclosure also provides a HER2/4-1BB bispecific fusion protein, such as one comprising the amino acid sequences set forth in SEQ ID NOs: 50 and 51, for use in treating a HER2-expressing tumor in a subject.

As disclosed herein, inventors have discovered new treatment methods, utilizing HER2/4-1BB bispecific fusion proteins comprising a lipocalin binding domain and an immunoglobulin binding domain that are safe and efficacious and achieve surprisingly beneficial clinical outcomes in patients suffering from HER2-expressing tumors. The present disclosure demonstrates that the described HER2/4-1BB bispecific fusion proteins administered in a pharmaceutical composition showed durable anti-tumor activity in a heavily pre-treated patient population across multiple tumor types, including those that are usually not responsive to immune therapy.

Additionally, the inventors have found that patients with lower numbers of CD8+ T cells in tumor tissue prior to treatment were responsive to treatment with HER2/4-1BB bispecific fusion proteins according to treatment regimens described herein, suggesting an improved alternative standard of care where a patient is non-responsive to other check point drugs.

Furthermore, the inventors have surprisingly found that the HER2/4-1BB bispecific fusion proteins disclosed herein are clinically active in patients with tumors that are characterized by a low expression of HER2.

The present disclosure demonstrates the effectiveness in humans in achieving clinical results that include, for example, an at least about 1.5-fold increase of CD8+ T cell numbers in the full tumor tissue; an at least about 1.5-fold increase of CD8+ T cell numbers in tumor cells; an at least about 1.5-fold increase of CD8+Ki67+ T cell numbers in the full tumor tissue; an at least about 1.5-fold increase of CD8+Ki67+ T cell numbers in tumor cells; an increase of CD8+ T cells from a pre-treatment level of less than about 500 per mm² of a measured area, wherein the measured area is an area of the full tumor tissue, tumor stroma, or tumor cells; an increase of the level of soluble 4-1BB (s4-1BB) in the blood serum; an at least 30% decrease in the target lesion; stable disease; a partial response; and a complete response.

The methods include, among other things, administering the disclosed HER2/4-1BB bispecific fusion proteins to a subject in a dose ranging from about 2.5 mg/kg to about 27 mg/kg. In some embodiments, the disclosed HER2/4-1BB bispecific fusion proteins may be administered once every week, once every two weeks, or once every three weeks. In some embodiments, the HER2/4-1BB bispecific fusion proteins are administered at a first dose and, subsequently, at a second dose, wherein the first dose exceeds the second dose.

### A. HER2/4-1BB Bispecific Fusion Protein of the Disclosure

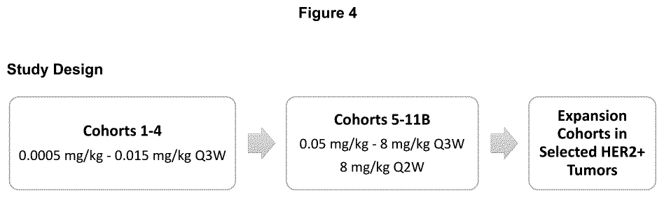

In some embodiments, a HER2/4-1BB bispecific fusion protein of the disclosure contains at least two subunits in any order: (1) a first subunit that comprises an antibody or an antigen-binding domain thereof specific for HER2, and (2) a second subunit that comprises a lipocalin mutein specific for 4-1BB (FIG. 4).

In some embodiments, a provided HER2/4-1BB bispecific fusion protein contains at least one additional subunit, for example, a third subunit. In some embodiments, a HER2/4-1BB fusion protein contains a third subunit that comprises a lipocalin mutein specific for 4-1BB.

In some embodiments, at least one subunit of a HER2/4-1BB bispecific fusion protein is fused at its N-terminus and/or its C-terminus to another subunit. In some embodiments, at least one subunit of a HER2/4-1BB bispecific fusion protein is fused to another subunit via a linker. A linker as described herein may be a peptide linker, for example, an unstructured glycine-serine (GS) linker, a glycosylated GS linker, or a proline-alanine-serine polymer (PAS) linker. In some embodiments, a (Gly₄Ser)₃ linker ((G₄S)₃) as shown in SEQ ID NO: 4 is used. Other exemplary linkers are shown in SEQ ID NOs: 5-14.

In some embodiments, the second subunit of a HER2/4-1BB bispecific fusion protein is linked via a linker, preferably a (G₄S)₃ linker, at its N-terminus to each of the C-terminus of the heavy chain constant region (CH) of the antibody or an antigen-binding domain thereof comprised in the first subunit (FIG. 4D).

In some embodiments, a lipocalin mutein subunit is fused to an antibody subunit of a provided HER2/4-1BB bispecific fusion protein via a peptide linker. In some embodiments, a lipocalin mutein subunit is fused, via a peptide linker, at its N-terminus or its C-terminus to an antibody subunit at the C-terminus of the antibody heavy chain (HC), the N-terminus of the HC, the C-terminus of the antibody light chain (LC), and/or the N-terminus of the LC (FIG. 4). In some preferred embodiment, a lipocalin mutein subunit is fused at its N-terminus to each of the HC of an antibody subunit of a HER2/4-1BB bispecific fusion protein via a peptide linker, preferably (G₄S)₃ linker (FIG. 4D).

In some embodiments, a provided HER2/4-1BB bispecific fusion protein comprises an antibody specific for HER2 fused at the C-terminus of both heavy chains to the N-terminus of a lipocalin mutein specific for 4-1BB.

In some embodiments, the Fc function of the Fc region of the antibody or an antigen-binding domain thereof comprised in the first subunit of a provided HER2/4-1BB bispecific fusion protein is preserved. Accordingly, a provided HER2/4-1BB bispecific fusion protein may be capable of binding Fc receptor-positive cell at the same time while simultaneously engaging 4-1BB and HER2. In some other embodiments, the Fc function of the Fc region of the antibody or an antigen-binding domain thereof comprised in the first subunit of a provided HER2/4-1BB bispecific fusion protein is reduced or fully suppressed, while the fusion protein is simultaneously engaging 4-1BB and HER2. In some embodiments, this may be achieved, for example, by switching from the IgG1 backbone to IgG4, as IgG4 is known to display reduced Fc-gamma receptor interactions compared to IgG1. In some embodiments, to further reduce the residual binding to Fc-gamma receptors, mutations may be introduced into the IgG4 backbone such as F234A and L235A. In some embodiments, an S228P mutation may also be introduced into the IgG4 backbone to minimize the exchange of IgG4 half-antibody (Silva et al., 2015). In some embodiments, F234A and L235A mutations may be introduced for decreased ADCC and ADCP (Glaesner et al., 2010) and/or M428L and N434S mutations or M252Y, S254T, and T256E mutations for extended serum half-life (Dall'Acqua et al., 2006, Zalevsky et al., 2010). In some embodiments, an additional N297A mutation may be present in the antibody heavy chain of a provided CD137/HER2 bispecific fusion protein in order to remove the natural glycosylation motif.

In some embodiments, the antibody or antigen-binding domain thereof comprised in a provided HER2/4-1BB bispecific fusion protein comprises the three heavy chain complementarity-determining regions (CDRs) shown in SEQ ID NO: 40, SEQ ID NO: 41, and SEQ ID NO: 42, and/or the three light chain CDRs shown in SEQ ID NO: 43, SEQ ID NO: 44, and SEQ ID NO: 45.

In some embodiments, the antibody or antigen-binding domain thereof comprised in a provided HER2/4-1BB bispecific fusion protein comprises a heavy chain variable region (HCVR) shown in SEQ ID NO: 46, and/or a light chain variable region (LCVR) shown in SEQ ID NO: 47.

In some embodiments, the antibody or antigen-binding domain thereof comprised in a provided HER2/4-1BB bispecific fusion protein comprises a heavy chain shown in SEQ ID NO: 49, and/or a light chain shown in SEQ ID NO: 50.

In some embodiments, the antibody or antigen-binding domain thereof comprised in a provided HER2/4-1BB bispecific fusion protein has a HCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 46, and/or a LCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 47. In other embodiments, the antibody or antigen-binding domain thereof comprised in a provided HER2/4-1BB bispecific fusion protein has a heavy chain with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 49, and/or a light chain with at least 70%, at least 75%, at least 80%, at least 85%, at least 90%, at least 92%, at least 95%, at least 97%, at least 98%, at least 99%, or even higher sequence identity to the amino acid sequence shown in SEQ ID NO: 50.

In some embodiments, the antibody or antigen-binding domain thereof comprised in a provided HER2/4-1BB bispecific fusion protein is an anti-HER2 antibody. In some embodiments, the antibody or antigen-binding domain thereof comprised in a provided HER2/4-1BB bispecific fusion protein is trastuzumab. In some embodiments, the antibody or antigen-binding domain thereof comprised in a provided HER2/4-1BB bispecific fusion protein is trastuzumab with an IgG4 backbone.

In some embodiments, the lipocalin mutein comprised in a provided HER2/4-1BB bispecific fusion protein is a mutein of mature human neutrophil gelatinase-associated lipocalin (hNGAL) having binding specificity for 4-1BB. A mutein of mature hNGAL may be designated herein as an “hNGAL mutein”.

In some embodiments, the lipocalin mutein comprised in a provided HER2/4-1BB bispecific fusion protein is capable of binding human 4-1BB with high affinity and/or co-stimulating human T cells when immobilized on a plastic dish together with an anti-CD3 antibody. In some embodiments, the lipocalin mutein comprised in a provided HER2/4-1BB bispecific fusion protein comprises an amino acid sequence selected from the group consisting of SEQ ID NOs: 21-39 or of a fragment or variant thereof. In some embodiments, the lipocalin mutein comprised in a provided HER2/4-1BB bispecific fusion protein has the amino acid sequence shown in SEQ ID NO: 22. In some embodiments, the lipocalin mutein comprised in a provided HER2/4-1BB bispecific fusion protein has an amino acid sequence with high sequence identity, such as at least 70%, at least 75%, at least 80%, at least 82%, at least 85%, at least 87%, at least 90%, at least 95%, at least 98%, at least 99%, or higher identity, to an amino acid sequence selected from the group consisting of SEQ ID NOs: 21-39. In some embodiments the lipocalin mutein comprised in a provided HER2/4-1BB bispecific fusion protein has an amino acid sequence with high sequence identity, such as at least 70%, at least 75%, at least 80%, at least 82%, at least 85%, at least 87%, at least 90%, at least 95%, at least 98%, at least 99%, or higher identity, to the amino acid sequence shown in SEQ ID NOs: 22.

In some embodiments, a provided HER2/4-1BB bispecific fusion protein is generated by genetic fusion of a 4-1BB-specific hNGAL mutein to a trastuzumab IgG4 variant, joined by a flexible, non-immunogenic peptide linker.

In some embodiments, a provided HER2/4-1BB bispecific fusion protein comprises the sets of amino acid sequences selected from the group consisting of SEQ ID NOs: 50 and 51, SEQ ID NOs: 50 and 53, SEQ ID NOs: 52 and 49, and SEQ ID NOs: 54 and 49.

In some embodiments, a provided HER2/4-1BB bispecific fusion protein comprises amino acid sequences having at least 70%, at least 75%, at least 80%, at least 85%, at least 90%, at least 92%, at least 95%, at least 97%, at least 98%, at least 99%, or higher sequence identity to the amino acid sequences shown in SEQ ID NOs: 50 and 51, SEQ ID NOs: 50 and 53, SEQ ID NOs: 52 and 49, and SEQ ID NOs: 54 and 49. In some embodiments, where a provided HER2/4-1BB bispecific fusion protein comprises more than one amino acid chain, a given value for the sequence identity relates to the average sequence identity normalized by the number of amino acid residues in both amino acid chains. For example, if a fusion protein consists of amino acid chain A having 100 amino acids and amino acid chain B having 50 amino acids, and another fusion protein consists of amino acid chain A′ having 100 amino acids 80% sequence identity to amino acid chain A and amino acid chain B′ having 50 amino acids and 95% sequence identity to amino acid chain B′, the average sequence identity between both fusion proteins will be (100/(100+50))×80%+(50/(100+50))×95%=85% sequence identity. In some preferred embodiments, where a fusion protein comprises more than one amino acid chain, a given value for the sequence identity means that a protein of interest comprises an amino acid sequence that has at least the given value of sequence identity to one chain of the bispecific fusion protein and comprises an amino acid sequence that has at least the given value of sequence identity to the other chain of the bispecific fusion protein.

In some embodiments, a provided HER2/4-1BB bispecific fusion protein is capable of engaging HER2 and 4-1BB simultaneously. In some embodiments, a provided fusion protein is capable of inducing 4-1BB clustering and signaling in a HER2-dependent manner. In some embodiments, a provided fusion protein is capable of activating 4-1BB signaling in a HER2-expressing tumor microenvironment. In some embodiments, a provided fusion protein is capable of co-stimulating T cell responses and/or enhancing T cell functions in a HER2-expressing tumor microenvironment.

In some embodiments, a provided HER2/4-1BB bispecific fusion protein comprises the amino acid sequences shown in SEQ ID NOs: 50 and 51. In some embodiments, a provided HER2/4-1BB bispecific fusion protein comprises two chains having the amino acid sequence shown in SEQ ID NO: 50 and two chains having the amino acid sequence shown in SEQ ID NO: 51.

In some embodiments, a provided HER2/4-1BB bispecific fusion protein comprising the amino acid sequences shown in SEQ ID NOs: 50 and 51 is capable of stimulating T cell responses in the presence of HER2-expressing tumor cells. In some embodiments, a provided HER2/4-1BB bispecific fusion protein comprising the amino acid sequences shown in SEQ ID NOs: 50 and 51 is capable of inducing IL-2 production in the presence of HER2-expressing tumor cells. In a specific embodiment, a HER2/4-1BB bispecific fusion of the disclosure induces IL-2 production in the presence of HER2-positive NCI-N87 cells with a potency (EC₅₀) of about 35 pmol/L. In some embodiments, a provided HER2/4-1BB bispecific fusion protein comprising the amino acid sequences shown in SEQ ID NOs: 50 and 51 is capable of inducing 4-1BB clustering and downstream signalling in the presence of HER2-expressing tumor cells. In a specific embodiment, a HER2/4-1BB bispecific fusion of the disclosure induces 4-1BB clustering and downstream signaling in a Jurkat NF-κB reporter cell line in the presence of HER2-expressing cells with a potency (EC₅₀) of about 50 pmol/L. The stimulation of T cell responses by provided fusion proteins in the presence of tumor cells may be assessed, for example, in an in-vitro T cell activation assay essentially described in Example 1.

In some embodiments, a provided HER2/4-1BB bispecific fusion protein comprising the amino acid sequences shown in SEQ ID NOs: 50 and 51 may have one or more anti-tumor effects in a subject following intravenous administration. The one or more anti-tumor effects may be decrease in target lesion, reduction of tumor size, suppression of tumor growth, delayed tumor recurrence, and/or improved overall survival.

In some embodiments, a provided HER2/4-1BB bispecific fusion protein comprising the amino acid sequences shown in SEQ ID NOs: 50 and 51 may decrease target lesion in a subject following intravenous administration. In some embodiments, the target lesion may be decreased by about 2%, about 5%, about 10%, about 15%, about 20%, about 25%, about 30%, about 35%, about 40%, about 45%, about 50%, about 55%, about 60%, about 70%, about 80%, about 90%, or about 100%.

In some embodiments, a provided HER2/4-1BB bispecific fusion protein comprising the amino acid sequences shown in SEQ ID NOs: 50 and 51 is capable of stimulating CD8+ T cell expansion in a subject following intravenous administration, preferably in the tumor microenvironment. In some embodiments, the increase of CD8+ T cells in the subject administered with the provided fusion protein may be observed in full tumor tissue, tumor cells, and/or tumor stroma. In some embodiments, the CD8+ T cell numbers in the subject administered with the provided fusion protein may be increased by about 1.1, about 1.2, about 1.3, about 1.4, about 1.5, about 2, about 2.5, about 3, about 4, about 5, about 6, about 7, about 8, about 9, about 10, or even more folds. The CD8+ T cell numbers may be increased by about 100, about 200, about 300, about 400, about 500, about 600, about 700, about 800, about 1000, or even more per mm² of measured area. In some embodiments, the CD8+ T cell numbers in the subject administered with the provided fusion protein may increase from a pre-treatment level of less than about 500, less than about 250, less than about 100, less than about 50 cells per mm² of measured area. The measured area may be full tumor tissue, tumor cells, or tumor stroma. The increase of CD8+ T cells may be more pronounced in the tumor cells than in full tumor tissue and/or tumor stroma.

In some embodiments, a provided HER2/4-1BB bispecific fusion protein comprising the amino acid sequences shown in SEQ ID NOs: 50 and 51 is capable of stimulating CD8+Ki67+ T cell proliferation and/or expansion in a subject following intravenous administration, preferably in the tumor microenvironment. In some embodiments, the increase of CD8+Ki67+ T cells in the subject administered with the provided fusion protein may be observed in full tumor tissue, tumor cells, and/or tumor stroma. The increase of CD8+Ki67+ T cells may be more pronounced in the tumor cells than in full tumor tissue and/or tumor stroma. In some embodiments, the CD8+Ki67+ T cell numbers in the subject administered with the provided fusion protein may be increased by about 1.1, about 1.2, about 1.3, about 1.4, about 1.5, about 2, about 2.5, about 3, about 4, about 5, about 6, about 7, about 8, about 9, about 10, or even more folds in full tumor tissue, tumor cells, and/or tumor stroma.

In some embodiments, a provided HER2/4-1BB bispecific fusion protein comprising the amino acid sequences shown in SEQ ID NOs: 50 and 51 is capable of stimulating tumor-infiltrating lymphocyte (TIL) proliferation and/or expansion in a subject following intravenous administration, preferably in the tumor microenvironment. In some embodiments, the increase of TILs in the subject administered with the provided fusion protein may be observed in full tumor tissue, tumor cells, and/or tumor stroma. In some embodiments, the TILs in the subject administered with the provided fusion protein may be increased by about 1.1, about 1.2, about 1.3, about 1.4, about 1.5, about 2, about 2.5, about 3, about 4, about 5, about 6, about 7, about 8, about 9, about 10, or even more folds. The increase of TILs may be more pronounced in the tumor cells than in full tumor tissue and/or tumor stroma. TILs include, but are not limited to, CD8+ T cells, CD4+ T cells, natural killer cells, and B cells.

In some embodiments, a provided HER2/4-1BB bispecific fusion protein comprising the amino acid sequences shown in SEQ ID NOs: 50 and 51 is capable of inducing changes in biomarker levels in a subject following intravenous administration. In some embodiments, a provided fusion protein may decrease the level of a biomarker in a subject. In some embodiments, a provided fusion protein may increase the level of a biomarker in a subject. The biomarker may be, for example, CD4, CD8, PD-L1, Ki67, (soluble) CD137 (4-1BB), HER2, IL-8, and FoxP3.

In some embodiments, a provided HER2/4-1BB bispecific fusion protein comprising the amino acid sequences shown in SEQ ID NOs: 50 and 51 is capable of increasing the level of soluble 4-1BB (s4-1BB) in a subject following intravenous administration. In some embodiments, s4-1BB is circulating s4-1BB. In some embodiments, the level of s4-1BB is increased in the blood serum of the subject. In some embodiments, the level of s4-1BB in the subject administered with the provided fusion protein may be increased by about 1.1, about 1.2, about 1.3, about 1.4, about 1.5, about 2, about 2.5, about 3, about 4, about 5, about 6, about 7, about 8, about 9, about 10 or even more folds. In some embodiments, the level of s4-1BB in the subject administered with the provided fusion protein may be increased to a concentration of about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml blood serum. In some embodiments, the level of s4-1BB in the subject administered with the provided fusion protein may be increased by about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml blood serum.

In some embodiments, a provided HER2/4-1BB bispecific fusion protein comprising the amino acid sequences shown in SEQ ID NOs: 50 and 51 may have a half-life of from about 10 hours to about 110 hours in a subject following intravenous administration. In some embodiments, a provided HER2/4-1BB bispecific fusion protein comprising the amino acid sequences shown in SEQ ID NOs: 50 and 51 may have a half-life of at least about 10 hours, at least about 14 hours, at least about 20 hours, at least about 50 hours, at least about 60 hours, at least about 70 hours, at least about 100 hours, at least about 105 hours, at least about 110 hours, or even longer in a subject following intravenous administration. In a specific embodiment, a provided HER2/4-1BB bispecific fusion protein comprising the amino acid sequences shown in SEQ ID NOs: 50 and 51 may have a half-life of at least about 72 hours in a subject following intravenous administration. In a specific embodiment, a provided HER2/4-1BB bispecific fusion protein comprising the amino acid sequences shown in SEQ ID NOs: 50 and 51 may have a half-life of at least about 104 hours in a subject following intravenous administration. The half-life values are based on the data provided in Example 3, taking into account the standard deviation.

In some embodiments, the peak serum concentration (Cₘₐₓ) of a provided HER2/4-1BB bispecific fusion protein comprising the amino acid sequences shown in SEQ ID NOs: 50 and 51 following intravenous administration to a subject may be from about 0.08 μg/mL to about 150 μg/ml. The Cₘₐₓ values are based on the data provided in Example 3, taking into account the standard deviation.

In some embodiments, the serum concentration over time (AUC_{inf}) of a provided HER2/4-1BB bispecific fusion protein comprising the amino acid sequences shown in SEQ ID NOs: 50 and 51 following intravenous administration to a subject may be from about 20 μg×h/mL to about 24000 μg×h/mL. The AUC_{inf} values are based on the data provided in Example 3, taking into account the standard deviation.

In some embodiments, a provided HER2/4-1BB bispecific fusion protein comprising the amino acid sequences shown in SEQ ID NOs: 50 and 51 may be administered to a subject, e.g., a mammal such as a human. In some embodiments, a subject administered with the provided fusion protein may have a HER2-expressing advanced or metastatic tumor. In some embodiments, a subject administered with the provided fusion protein may have gastric cancer (e.g., gastric adenocarcinoma), gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer (e.g., rectal adenocarcinoma), colon cancer, pancreatic cancer, biliary tract cancer, salivary duct cancer, bladder cancer, and/or cancer of unknown primary. In some embodiments, the subject may have gastric cancer. In some embodiments, the subject may have gastroesophageal cancer, preferably gastroesophageal junction cancer. In some embodiments, the subject may have gastric or gastroesophageal junction adenocarcinoma. In some embodiments, the subject may have colorectal cancer. In some embodiments, the subject may have lung cancer, preferably non-small cell lung cancer.

In some embodiments, a provided HER2/4-1BB bispecific fusion protein comprising the amino acid sequences shown in SEQ ID NOs: 50 and 51 may be administered to a previously treated subject. The subject administered with the provided fusion protein may have been previously treated with chemotherapy, a HER2-targeting drug, a 4-1BB/4-1BBL pathway-targeting drug, a PD-1 signaling pathway-targeting drug, a CTLA-4 signaling pathway-targeting drug, or combinations of any of the foregoing (e.g., a combination of chemotherapy and a HER2-targeting drug). A HER2-targeting drug may be an anti-HER2 antibody, such as trastuzumab or pertuzumab. A 4-1BB/4-1BBL pathway-targeting drug may be an anti-4-1BB antibody, such as urelumab or utomilumab. In some embodiments, the subject has not been previously treated with a PD-1 signaling pathway-targeting drug. In some embodiments, the treatment with the HER2/4-1BB bispecific fusion protein does not comprise a (co-)treatment with a PD-1 signaling pathway-targeting drug. A PD-1 signaling pathway-targeting drug may be an anti-PD-1 antibody, such as nivolumab, pembrolizumab, or cemiplimab. A CTLA-4 signaling pathway-targeting drug may be an anti-CTLA-4 antibody, such as ipilimumab. In some embodiments, the treatment with the HER2/4-1BB bispecific fusion protein provided herein comprises (co-)treatment with a PD-1 signaling pathway-targeting drug (or PD-1 axis inhibitor), such as an antibody specific for PD-1 (e.g., nivolumab, pembrolizumab, cemiplimab or tislelizumab) or PD-L1 (e.g., atezolizumab, avelumab, durvalumab or BMS-936559, preferably atezolizumab), e.g., as described in WO 2020/043683, which is herein incorporated by reference in its entirety.

In some embodiments, the subject administered with the provided fusion protein may have been previously treated with chemotherapy, a HER2-targeting drug or a combination thereof. In some particular embodiments, the subject may have been previously treated with a platinum, fluoropyrimidine and a HER2-targeting drug. Preferably, the HER2-targeting drug is an anti-HER2 antibody, wherein, more preferably, the anti-HER2 antibody is trastuzumab.

In some embodiments, a provided HER2/4-1BB bispecific fusion protein comprising the amino acid sequences shown in SEQ ID NOs: 50 and 51 may be administered to a subject that has been pre-treated with a B cell depleting agent. In some embodiments, the B cell depleting agent may be an anti-CD20 antibody, such as rituximab, obinutuzumab, ocrelizumab, or veltuzumab.

In some embodiments, a provided HER2/4-1BB bispecific fusion protein comprising the amino acid sequences shown in SEQ ID NOs: 50 and 51 may be administered to a subject that has been pre-treated with obinutuzumab. In some embodiments, obinutuzumab is administered to the subject about seven days before the provided HER2/4-1BB bispecific fusion protein comprising the amino acid sequences shown in SEQ ID NOs: 50 and 51 is first administered to the subject. In some embodiments, obinutuzumab is administered to the subject at a dose of about 1000 mg to about 2000 mg. In some embodiments, obinutuzumab is administered to the subject at a dose of about 2000 mg seven days before the provided HER2/4-1BB bispecific fusion protein comprising the amino acid sequences shown in SEQ ID NOs: 50 and 51 is first administered to the subject. In some embodiments, obinutuzumab is administered to the subject at a dose of 1000 mg seven days before and six days before the provided HER2/4-1BB bispecific fusion protein comprising the amino acid sequences shown in SEQ ID NOs: 50 and 51 is first administered to the subject.

In some embodiments, a provided HER2/4-1BB bispecific fusion protein comprising the amino acid sequences shown in SEQ ID NOs: 50 and 51 may be administered as an adjuvant.

In some embodiments, a provided HER2/4-1BB bispecific fusion protein comprising the amino acid sequences shown in SEQ ID NOs: 50 and 51 may be administered to a subject who has a pre-treatment level of less than about 1000, less than about 750, less than about 500, less than about 400, less than about 400, less than about 300, less than about 250, less than about 200, less than about 150, less than about 100, less than about 90, less than about 80, less than about 70, less than about 60, less than about 50, less than about 45, less than about 40, less than about 35, or even lower CD8+ T cells per mm² tumor tissue. In some embodiments, a provided HER2/4-1BB bispecific fusion protein comprising the amino acid sequences shown in SEQ ID NOs: 50 and 51 may be administered to a subject who has a pre-treatment level of less than about 250 CD8+ T cells per mm² tumor tissue.

In some embodiments, a provided HER2/4-1BB bispecific fusion protein comprising the amino acid sequences shown in SEQ ID NOs: 50 and 51 may be administered to a subject who has a pre-treatment level of less than about 50%, less than about 40%, less than about 30%, less than about 25%, less than about 20%, less than about 15%, less than about 10%, less than about 5%, or even lower PD-L1+ cells of total immune cells. In some embodiments, a provided HER2/4-1BB bispecific fusion protein comprising the amino acid sequences shown in SEQ ID NOs: 50 and 51 may be administered to a subject who has a pre-treatment level of less than about 25% PD-L1+ cells of total immune cells.

In some embodiments, a provided HER2/4-1BB bispecific fusion protein comprising the amino acid sequences shown in SEQ ID NOs: 50 and 51 may be administered to a subject who has a pre-treatment level of less than about 250 CD8+ T cells per mm² tumor tissue and a pre-treatment level of less than about 25% PD-L1+ cells of total immune cells.

In some embodiments, the HER2-expressing tumor is a HER2-positive (HER2+) tumor. In some embodiments, the tumor is characterized by a HER2 status of IHC3+, IHC2+/(F)ISH+ or (F)ISH+, preferably IHC3+ or IHC2+/(F)ISH+. In some embodiments, the tumor exhibits HER2 gene amplification.

In some embodiments, the HER2-expressing tumor is characterized by a low expression of HER2. In some embodiments, the tumor is characterized by a HER2 status of IHC1+ or IHC2+/(F)ISH−. In some embodiments, the tumor does not exhibit HER2 gene amplification.

In some embodiments, a provided HER2/4-1BB bispecific fusion protein comprising the amino acid sequences shown in SEQ ID NOs: 50 and 51 may induce anti-drug antibodies (ADA) in a subject following intravenous administration. In some embodiments, ADA may be detected in a subject following intravenous administration of the provided fusion protein at a dose level from about 0.05 mg/kg to about 27 mg/kg. In some embodiments, ADA may be detected in a subject following intravenous administration of the provided fusion protein at dose levels of about 0.05 mg/kg, about 0.15 mg/kg, about 0.5 mg/kg, about 1 mg/kg, about 2.5 mg/kg, about 5 mg/kg, about 8 mg/kg, about 12 mg/kg, about 18 mg/kg, about 27 mg/kg, or higher. In some embodiments, ADA may be detected in a subject after the first does, after one treatment cycle, after two treatment cycles, after three treatment cycles, or even later, of the provided fusion protein.

In some other embodiments, a provided HER2/4-1BB bispecific fusion protein comprising the amino acid sequences shown in SEQ ID NOs: 50 and 51 does not induce ADA in a subject following intravenous administration.

In some embodiments, a provided HER2/4-1BB comprising the amino acid sequences shown in SEQ ID NOs: 50 and 51 bispecific fusion protein may have favorable safety profile to permit a dose level of about 0.0005 mg/kg, about 0.0015 mg/kg, about 0.005 mg/kg, about 0.015 mg/kg, about 0.05 mg/kg, about 0.15 mg/kg, about 0.5 mg/kg, about 1 mg/kg, about 2.5 mg/kg, about 5.0 mg/kg, about 8 mg/kg, about 12 mg/kg, about 18 mg/kg, about 27 mg/kg, or even higher. In some embodiments, a provided HER2/4-1BB comprising the amino acid sequences shown in SEQ ID NOs: 50 and 51 bispecific fusion protein may permit a dose level of about 2.5 mg/kg, about 5.0 mg/kg, about 8 mg/kg, about 12 mg/kg, about 18 mg/kg, about 27 mg/kg, or even higher. In some embodiments, a provided HER2/4-1BB comprising the amino acid sequences shown in SEQ ID NOs: 50 and 51 bispecific fusion protein may permit a dose level of about 8 mg/kg, about 12 mg/kg, about 18 mg/kg, about 27 mg/kg, or even higher.

In some embodiments, a provided HER2/4-1BB bispecific fusion protein comprising the amino acid sequences shown in SEQ ID NOs: 50 and 51 may have favorable pharmacokinetic properties to permit a dosing schedule of about once every week, about once every two weeks, about once every three weeks, or about once every four weeks. In some embodiments, a HER2/4-1BB bispecific fusion protein of the disclosure may permit a dosing schedule of about twice a week, about once a week, about once every ten days, about once every two weeks, about once every three weeks, about once every four weeks, about once every five weeks, about once every month, about once every six weeks, about once every seven weeks, about once every eight weeks, or about once every two months. In some embodiments, a HER2/4-1BB bispecific fusion protein of the disclosure may permit a dosing schedule of about once a week, about once every two weeks, or about once every three weeks. In some embodiments, a HER2/4-1BB bispecific fusion protein may provide superior tumor response, such as a longer duration of response, when administered following a dosing schedule of every two weeks as compared to a dosing schedule of every three weeks.

In some embodiments, a provided HER2/4-1BB bispecific fusion protein comprising the amino acid sequences shown in SEQ ID NOs: 50 and 51 may be administered to a subject at a dose of from about 2.5 mg/kg to about 27 mg/kg at an interval of about once every two weeks to about once every week, at a dose of from about 5 mg/kg to about 27 mg/kg at an interval of about once every two weeks to about once every week, at a dose of from about 8 mg/kg to about 27 mg/kg at an interval of about once every two weeks to about once every week, at a dose of from about 2.5 mg/kg to about 12 mg/kg at an interval of about once every two weeks to about once every week, at a dose of from about 5 mg/kg to about 12 mg/kg at an interval of about once every two weeks to about once every week, or at a dose of from about 8 mg/kg to about 18 mg/kg at an interval of about once every two weeks to about once every week.

In some embodiments, a provided HER2/4-1BB bispecific fusion protein comprising the amino acid sequences shown in SEQ ID NOs: 50 and 51 may be administered to a subject at a dose of about 2.5 mg/kg at an interval of about once every two weeks to about once every week, at a dose of about 5 mg/kg at an interval of about once every two weeks to about once every week, at a dose of about 8 mg/kg at an interval of about once every two weeks to about once every week, at a dose of about 12 mg/kg at an interval of about once every two weeks to about once every week, at a dose of about 18 mg/kg at an interval of about once every two weeks to about once every week, or at a dose of about 27 mg/kg at an interval of about once every two weeks to about once every week.

In some embodiments, a provided HER2/4-1BB bispecific fusion protein comprising the amino acid sequences shown in SEQ ID NOs: 50 and 51 may be administered to a subject at a dose of from about 2.5 mg/kg to about 27 mg/kg at an interval of about once every two weeks, at a dose of from about 5 mg/kg to about 27 mg/kg at an interval of about once every two weeks, at a dose of from about 8 mg/kg to about 27 mg/kg at an interval of about once every two weeks, at a dose of from about 2.5 mg/kg to about 12 mg/kg at an interval of about once every two weeks, at a dose of from about 5 mg/kg to about 12 mg/kg at an interval of about once every two weeks, or at a dose of from about 8 mg/kg to about 18 mg/kg at an interval of about once every two weeks.

In some embodiments, a provided HER2/4-1BB bispecific fusion protein comprising the amino acid sequences shown in SEQ ID NOs: 50 and 51 may be administered to a subject at a dose of about 2.5 mg/kg, about 5 mg/kg, about 8 mg/kg, about 12 mg/kg, about 18 mg/kg, or about 27 mg/kg at an interval of about once every two weeks.

In some embodiments, a provided HER2/4-1BB bispecific fusion protein comprising the amino acid sequences shown in SEQ ID NOs: 50 and 51 may be administered to a subject at a dose of from about 2.5 mg/kg to about 27 mg/kg at an interval of about once every week, at a dose of from about 5 mg/kg to about 27 mg/kg at an interval of about once every week, at a dose of from about 8 mg/kg to about 27 mg/kg at an interval of about once every week, at a dose of from about 2.5 mg/kg to about 12 mg/kg at an interval of about once every week, at a dose of from about 5 mg/kg to about 12 mg/kg at an interval of about once every week, or at a dose of from about 8 mg/kg to about 18 mg/kg at an interval of about once every week.

In some embodiments, a provided HER2/4-1BB bispecific fusion protein comprising the amino acid sequences shown in SEQ ID NOs: 50 and 51 may be administered to a subject at a dose of about 2.5 mg/kg, about 5 mg/kg, about 8 mg/kg, about 12 mg/kg, about 18 mg/kg, or about 27 mg/kg at an interval of about once every week.

In some embodiments, a provided HER2/4-1BB bispecific fusion protein comprising the amino acid sequences shown in SEQ ID NOs: 50 and 51 may be administered to a subject at a dose that results in 20 μg/mL serum concentration of the fusion protein. In some embodiments, the dose that results in 20 μg/mL serum concentration of the fusion protein may be a dose of about 2.5 mg/kg, about 5 mg/kg, about 8 mg/kg, about 12 mg/kg, about 18 mg/kg, or about 27 mg/kg. The does may be administered at an interval of about once every week, about once every two weeks, or about once every three weeks.

In some embodiments, a provided HER2/4-1BB bispecific fusion protein comprising the amino acid sequences shown in SEQ ID NOs: 50 and 51 is not associated with dose limiting toxicity after being administered to a subject. In some embodiments, a provided HER2/4-1BB bispecific fusion protein comprising the amino acid sequences shown in SEQ ID NOs: 50 and 51 is not associated with dose limiting toxicity after being administered to a subject when administered to a subject at a dose of up to about 2.5 mg/kg, up to about 5 mg/kg, up to about 8 mg/kg, up to about 12 mg/kg, up to about 18 mg/kg, or up to about 27 mg/kg at an interval of about once every three weeks. In some embodiments, a provided HER2/4-1BB bispecific fusion protein comprising the amino acid sequences shown in SEQ ID NOs: 50 and 51 is not associated with dose limiting toxicity after being administered to a subject when administered to a subject at a dose of up to about 2.5 mg/kg, up to about 5 mg/kg, up to about 8 mg/kg, up to about 12 mg/kg, up to about 18 mg/kg, or up to about 27 mg/kg at an interval of about once every two weeks. In some embodiments, a provided HER2/4-1BB bispecific fusion protein comprising the amino acid sequences shown in SEQ ID NOs: 50 and 51 is not associated with dose limiting toxicity after being administered to a subject when administered to a subject at a dose of up to about 2.5 mg/kg, up to about 5 mg/kg, up to about 8 mg/kg, up to about 12 mg/kg, up to about 18 mg/kg, or up to about 27 mg/kg at an interval of about once every week.

In some embodiments, a HER2/4-1BB bispecific fusion protein provided herein, e.g., comprising the amino acid sequences shown in SEQ ID NOs: 50 and 51, may be administered to a subject at a first dose and, subsequently, at a second dose, wherein the first dose exceeds the second dose. In some embodiments, the fusion protein may be administered at the first dose up to five times, up to four times, up to three times or up to two times. In some embodiments, the fusion protein may be administered two times at the first dose.

In some embodiments, the fusion protein may be administered at an interval of about once every three weeks, about once every two weeks, or about once every week. In some embodiments, the fusion protein may be administered at an interval of about once every week. In some embodiments, the fusion protein may be administered at an interval of about once every two weeks. In some embodiments, the fusion protein may be administered at an interval of about once every three weeks.

In some embodiments, the first dose may be from about 5 mg/kg to about 27 mg/kg. In some embodiments, the first dose may be from about 12 mg/kg to about 27 mg/kg. In some embodiments, the first dose may be about 18 mg/kg. In some embodiments, the first dose may be about 12 mg/kg.

In some embodiments, the second dose may be from about 2.5 mg/kg to about 18 mg/kg. In some embodiments, the second dose may be from about 2.5 mg/kg to about 12 mg/kg. In some embodiments, the second dose may be about 8 mg/kg. In some embodiments, the second dose may be about 5 mg/kg. In some embodiments, the second dose may be about 2.5 mg/kg.

In some embodiments, the first dose may be about 18 mg/kg, and the second dose may be about 8 mg/kg. In some embodiments, the first dose may be about 18 mg/kg, and the second dose may be about 5 mg/kg. In some embodiments, the first dose may be about 18 mg/kg, and the second dose may be about 2.5 mg/kg. In some embodiments, the first dose may be about 12 mg/kg, and the second dose may be about 8 mg/kg. In some embodiments, the first dose may be about 12 mg/kg, and the second dose may be about 5 mg/kg. In some embodiments, the first dose may be about 12 mg/kg, and the second dose may be about 2.5 mg/kg.

In some embodiments, a provided HER2/4-1BB bispecific fusion protein comprising the amino acid sequences shown in SEQ ID NOs: 50 and 51 may be administered to a subject by infusion. In some embodiments, a provided HER2/4-1BB bispecific fusion protein comprising the amino acid sequences shown in SEQ ID NOs: 50 and 51 may be administered to a subject by intravenous infusion.

In some embodiments, a provided HER2/4-1BB bispecific fusion protein comprising the amino acid sequences shown in SEQ ID NOs: 50 and 51 may be used as an anti-tumor agent, an anti-infection agent, an anti-inflammatory agent, and/or an immune modulator.

A HER2/4-1BB bispecific fusion protein of the disclosure may be used in a method provided herein.

### B. Methods of the Disclosure

In some embodiments, the present disclosure provides a method for treating a tumor, particularly a HER2-expressing tumor, in a subject, wherein the method comprises administering a therapeutically effective amount of a provided HER2/4-1BB bispecific fusion protein comprising the amino acid sequences shown in SEQ ID NOs: 50 and 51. The provided HER2/4-1BB bispecific fusion protein comprising the amino acid sequences shown in SEQ ID NOs: 50 and 51 may be administered at a dose of from about 2.5 mg/kg to about 27 mg/kg. The provided HER2/4-1BB bispecific fusion protein comprising the amino acid sequences shown in SEQ ID NOs: 50 and 51 may be administered once every week, once every two weeks, or once every three weeks. The subject may have gastric cancer (e.g., gastric adenocarcinoma), gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer (e.g., rectal adenocarcinoma), colon cancer, pancreatic cancer, biliary tract cancer, salivary duct cancer, bladder cancer, and/or cancer of unknown primary. In some embodiments, the subject may have gastric cancer. In some embodiments, the subject may have gastroesophageal cancer, preferably gastroesophageal junction cancer. In some embodiments, the subject may have gastric or gastroesophageal junction adenocarcinoma. In some embodiments, the subject may have colorectal cancer. In some embodiments, the subject may have lung cancer, preferably non-small cell lung cancer.

In some embodiments, the present disclosure provides a HER2/4-1BB bispecific fusion protein comprising the amino acid sequences shown in SEQ ID NOs: 50 and 51 for use in treating a tumor, particularly a HER2-expressing tumor, in a subject, comprising administering a therapeutically effective amount of a provided HER2/4-1BB bispecific fusion protein comprising the amino acid sequences shown in SEQ ID NOs: 50 and 51. The provided HER2/4-1BB bispecific fusion protein comprising the amino acid sequences shown in SEQ ID NOs: 50 and 51 may be administered at a dose of from about 2.5 mg/kg to about 27 mg/kg. The provided HER2/4-1BB bispecific fusion protein comprising the amino acid sequences shown in SEQ ID NOs: 50 and 51 may be administered once every week, once every two weeks, or once every three weeks. The subject may have gastric cancer (e.g., gastric adenocarcinoma), gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer (e.g., rectal adenocarcinoma), colon cancer, pancreatic cancer, biliary tract cancer, salivary duct cancer, bladder cancer, and/or cancer of unknown primary. In some embodiments, the subject may have gastric cancer. In some embodiments, the subject may have gastroesophageal cancer, preferably gastroesophageal junction cancer. In some embodiments, the subject may have gastric or gastroesophageal junction adenocarcinoma. In some embodiments, the subject may have colorectal cancer. In some embodiments, the subject may have lung cancer, preferably non-small cell lung cancer.

In some embodiments, the present disclosure provides the use of a HER2/4-1BB bispecific fusion protein comprising the amino acid sequences shown in SEQ ID NOs: 50 and 51 for the manufacture of a medicament for use in treating a tumor, particularly a HER2-expressing tumor, in a subject, wherein the treatment comprises administering a therapeutically effective amount of a provided HER2/4-1BB bispecific fusion protein comprising the amino acid sequences shown in SEQ ID NOs: 50 and 51. The provided HER2/4-1BB bispecific fusion protein comprising the amino acid sequences shown in SEQ ID NOs: 50 and 51 may be administered at a dose of from about 2.5 mg/kg to about 27 mg/kg. The provided HER2/4-1BB bispecific fusion protein comprising the amino acid sequences shown in SEQ ID NOs: 50 and 51 may be administered once every week, once every two weeks, or once every three weeks. The subject may have gastric cancer (e.g., gastric adenocarcinoma), gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer (e.g., rectal adenocarcinoma), colon cancer, pancreatic cancer, biliary tract cancer, salivary duct cancer, bladder cancer, and/or cancer of unknown primary. In some embodiments, the subject may have gastric cancer. In some embodiments, the subject may have gastroesophageal cancer, preferably gastroesophageal junction cancer. In some embodiments, the subject may have gastric or gastroesophageal junction adenocarcinoma. In some embodiments, the subject may have colorectal cancer. In some embodiments, the subject may have lung cancer, preferably non-small cell lung cancer.

In some embodiments, the present disclosure provides a method for treating a HER2-expressing tumor in a subject, wherein the method comprises administering by intravenous infusion a provided HER2/4-1BB bispecific fusion protein comprising the amino acid sequences shown in SEQ ID NOs: 50 and 51 at least once every week at a dose of from about 2.5 mg/kg to about 27 mg/kg.

In some embodiments, the present disclosure provides a HER2/4-1BB bispecific fusion protein comprising the amino acid sequences shown in SEQ ID NOs: 50 and 51 for use in treating a HER2-expressing tumor in a subject, comprising administering by intravenous infusion the HER2/4-1BB bispecific fusion protein comprising the amino acid sequences shown in SEQ ID NOs: 50 and 51 at least once every week at a dose of from about 2.5 mg/kg to about 27 mg/kg.

In some embodiments, the present disclosure provides the use of a HER2/4-1BB bispecific fusion protein comprising the amino acid sequences shown in SEQ ID NOs: 50 and 51 for the manufacture of a medicament for use in treating a HER2-expressing tumor in a subject, wherein the treatment preferably comprises administering by intravenous the HER2/4-1BB bispecific fusion protein comprising the amino acid sequences shown in SEQ ID NOs: 50 and 51 at least once every week at a dose of from about 2.5 mg/kg to about 27 mg/kg.

In some embodiments, the HER2/4-1BB bispecific fusion protein comprising the amino acid sequences shown in SEQ ID NOs: 50 and 51 is administered to the subject at a dose of about 8 mg/kg, about 12 mg/kg, about 18 mg/kg, or about 27 mg/kg.

In some embodiments, the HER2/4-1BB bispecific fusion protein comprising the amino acid sequences shown in SEQ ID NOs: 50 and 51 is administered to the subject about once every week, about once about two weeks, about once about three weeks, or about once every four weeks.

In some embodiments, administering the HER2/4-1BB bispecific fusion protein comprising the amino acid sequences shown in SEQ ID NOs: 50 and 51 is sufficient to achieve one or more anti-tumor effects. For example, the administration of the fusion protein may decrease target lesion, reduce tumor size, suppress tumor growth, delay tumor recurrence, and/or improve overall survival.

In some embodiments, administering the HER2/4-1BB bispecific fusion protein comprising the amino acid sequences shown in SEQ ID NOs: 50 and 51 about once every two weeks achieves superior clinical response, such as a longer duration of response, as compared to administering the fusion protein about once every three weeks.

In some embodiments, administering the HER2/4-1BB bispecific fusion protein results in decreased target lesion in the subject. In some embodiments, administering the HER2/4-1BB bispecific fusion protein result in decreased target lesion in the subject by about 2%, about 5%, about 10%, about 15%, about 20%, about 25%, about 30%, about 35%, about 40%, about 45%, about 50%, about 55%, about 60%, about 70%, about 80%, about 90%, or about 100%.

In some embodiments, administering the HER2/4-1BB bispecific fusion protein results in CD8+ T cell proliferation and/or expansion in the subject, preferably in the tumor microenvironment. In some embodiments, the increase of CD8+ T cells in the subject administered with the provided fusion protein may be observed in full tumor tissue, tumor cells, and/or tumor stroma. In some embodiments, administering the HER2/4-1BB bispecific fusion protein result in increased CD8+ T cell numbers in the subject by about 1.1, about 1.2, about 1.3, about 1.4, about 1.5, about 2, about 2.5, about 3, about 4, about 5, about 6, about 7, about 8, about 9, about 10, or even more folds. In some embodiments, administering the HER2/4-1BB bispecific fusion protein result in increased CD8+ T cell numbers in the subject by about 100, about 200, about 300, about 400, about 500, about 600, about 700, about 800, about 1000, or even more per mm² of measured area. In some embodiments, administering the HER2/4-1BB bispecific fusion protein result in the increase of CD8+ T cell numbers in the subject from a pre-treatment level of less than about 500, less than about 250, less than about 100, less than about 50, or an even lower number of cells per mm² of measured area. The measured area may be full tumor tissue, tumor cells, or tumor stroma. In some embodiments, administering the HER2/4-1BB bispecific fusion protein result in more pronounced increase of CD8+ T cells in the tumor cells than in full tumor tissue and/or tumor stroma in the subject.

In some embodiments, administering the HER2/4-1BB bispecific fusion protein results in CD8+Ki67+ T cell proliferation and/or expansion in the subject, preferably in the tumor microenvironment. In some embodiments, the increase of CD8+Ki67+ T cells in the subject administered with the provided fusion protein may be observed in full tumor tissue, tumor cells, and/or tumor stroma. In some embodiments, administering the HER2/4-1BB bispecific fusion protein result in more pronounced increase of CD8+Ki67+ T cells in the tumor cells than in full tumor tissue and/or tumor stroma in the subject. In some embodiments, the CD8+Ki67+ T cell numbers in the subject administered with the provided fusion protein may be increased by about 1.1, about 1.2, about 1.3, about 1.4, about 1.5, about 2, about 2.5, about 3, about 4, about 5, about 6, about 7, about 8, about 9, about 10, or even more folds in full tumor tissue, tumor cells, and/or tumor stroma.

In some embodiments, administering the HER2/4-1BB bispecific fusion protein results in tumor-infiltrating lymphocyte (TIL) proliferation and/or expansion in the subject, preferably in the tumor microenvironment. In some embodiments, the increase of TILs in the subject administered with the provided fusion protein may be observed in full tumor tissue, tumor cells, and/or tumor stroma. In some embodiments, administering the HER2/4-1BB bispecific fusion protein result in increased TILs in the subject by about 1.1, about 1.2, about 1.3, about 1.4, about 1.5, about 2, about 2.5, about 3, about 4, about 5, about 6, about 7, about 8, about 9, about 10, or even more folds. In some embodiments, administering the HER2/4-1BB bispecific fusion protein result in more pronounced increase of TILs in the tumor cells than in full tumor tissue and/or tumor stroma in the subject.

In some embodiments, administering the HER2/4-1BB bispecific fusion protein results in decrease or increase in biomarker levels in the subject. The biomarker may be, for example, CD4, CD8, PD-L1, Ki67, (soluble) CD137 (4-1BB), HER2, IL-8, and FoxP3.

In some embodiments, administering the HER2/4-1BB bispecific fusion protein results in an increase of the level of soluble 4-1BB (s4-1BB) in the subject. In some embodiments, s4-1BB is circulating s4-1BB. In some embodiments, the level of s4-1BB is increased in the blood serum of the subject. In some embodiments, the level of s4-1BB may be increased by about 1.1, about 1.2, about 1.3, about 1.4, about 1.5, about 2, about 2.5, about 3, about 4, about 5, about 6, about 7, about 8, about 9, about 10 or even more folds. In some embodiments, the level of s4-1BB may be increased to a concentration of about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml blood serum. In some embodiments, the level of s4-1BB may be increased by about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml blood serum.

In some embodiments, administering the HER2/4-1BB bispecific fusion protein results in ≥20 μg/mL serum concentration of the fusion protein. In some embodiments, the fusion protein is administered at a dose level from about 2.5 mg/kg to about 27 mg/kg, such as about 2.5 mg/kg, about 5 mg/kg, about 8 mg/kg, about 12 mg/kg, about 18 mg/kg, or about 27 mg/kg. The fusion protein may be administered at an interval of about once every week, about once every two weeks, or about once every three weeks.

In some embodiments, administering the HER2/4-1BB bispecific fusion protein results in anti-drug antibodies (ADA) in the subject after the first does, after one treatment cycle, after two treatment cycles, after three treatment cycles, or even later. In some embodiments, the fusion protein is administered at a dose level from about 0.05 mg/kg to about 27 mg/kg, such as about 0.05 mg/kg, about 0.15 mg/kg, about 0.5 mg/kg, about 1 mg/kg, about 2.5 mg/kg, about 5 mg/kg, about 8 mg/kg, about 12 mg/kg, about 18 mg/kg, or about 27 mg/kg.

In some embodiments, administering the HER2/4-1BB bispecific fusion protein does not result in anti-drug antibodies (ADA) in the subject.

In some embodiments, administering the HER2/4-1BB bispecific fusion protein comprising the amino acid sequences shown in SEQ ID NOs: 50 and 51 is not associated with dose limiting toxicity after being administered to a subject. In some embodiments, a provided HER2/4-1BB bispecific fusion protein comprising the amino acid sequences shown in SEQ ID NOs: 50 and 51 is not associated with dose limiting toxicity after being administered to a subject when administered to a subject at a dose of up to about 2.5 mg/kg, up to about 5 mg/kg, up to about 8 mg/kg, up to about 12 mg/kg, up to about 18 mg/kg, or up to about 27 mg/kg at an interval of about once every three weeks. In some embodiments, a provided HER2/4-1BB bispecific fusion protein comprising the amino acid sequences shown in SEQ ID NOs: 50 and 51 is not associated with dose limiting toxicity after being administered to a subject when administered to a subject at a dose of up to about 2.5 mg/kg, up to about 5 mg/kg, up to about 8 mg/kg, up to about 12 mg/kg, up to about 18 mg/kg, or up to about 27 mg/kg at an interval of about once every two weeks. In some embodiments, a provided HER2/4-1BB bispecific fusion protein comprising the amino acid sequences shown in SEQ ID NOs: 50 and 51 is not associated with dose limiting toxicity after being administered to a subject when administered to a subject at a dose of up to about 2.5 mg/kg, up to about 5 mg/kg, up to about 8 mg/kg, up to about 12 mg/kg, up to about 18 mg/kg, or up to about 27 mg/kg at an interval of about once every week.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in one or more anti-tumor effects, such as decreased target lesion, reduced tumor size, suppressed tumor growth, delayed tumor recurrence, and/or improved overall survival.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in decreased target lesion in the subject by about 2%, about 5%, about 10%, about 15%, about 20%, about 25%, about 30%, about 35%, about 40%, about 45%, about 50%, about 55%, about 60%, about 70%, about 80%, about 90%, or about 100%. In some embodiments, the fusion protein is administered at least once every week at a dose of from about 2.5 mg/kg to about 27 mg/kg.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in CD8+ T cell proliferation and/or expansion in full tumor tissue, tumor cells, and/or tumor stroma in the subject, by about 1.1, about 1.2, about 1.3, about 1.4, about 1.5, about 2, about 2.5, about 3, about 4, about 5, about 6, about 7, about 8, about 9, about 10, or even more folds, or by about 100, about 200, about 300, about 400, about 500, about 600, about 700, about 800, about 1000, or even more per mm² of measured area, or from a pre-treatment level of less than about 500, less than about 250, less than about 100, less than about 50, or an even lower number of cells per mm² of measured area. The measured area may be full tumor tissue, tumor cells, or tumor stroma. In some embodiments, the fusion protein is administered at least once every week at a dose of from about 2.5 mg/kg to about 27 mg/kg.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in CD8+Ki67+ T cell proliferation and/or expansion in full tumor tissue, tumor cells, and/or tumor stroma in the subject. In some embodiments, the CD8+Ki67+ T cell numbers in the subject administered with the provided fusion protein may be increased by about 1.1, about 1.2, about 1.3, about 1.4, about 1.5, about 2, about 2.5, about 3, about 4, about 5, about 6, about 7, about 8, about 9, about 10, or even more folds in full tumor tissue, tumor cells, and/or tumor stroma. In some embodiments, the fusion protein is administered at least once every week at a dose of from about 2.5 mg/kg to about 27 mg/kg.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in tumor-infiltrating lymphocyte (TIL) proliferation and/or expansion in full tumor tissue, tumor cells, and/or tumor stroma in the subject, by about 1.1, about 1.2, about 1.3, about 1.4, about 1.5, about 2, about 2.5, about 3, about 4, about 5, about 6, about 7, about 8, about 9, about 10, or even more folds. In some embodiments, the fusion protein is administered at least once every week at a dose of from about 2.5 mg/kg to about 27 mg/kg.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in ≥20 μg/mL serum concentration of the fusion protein. In some embodiments, the fusion protein is administered at a dose level from about 2.5 mg/kg to about 27 mg/kg, such as about 2.5 mg/kg, about 5 mg/kg, about 8 mg/kg, about 12 mg/kg, about 18 mg/kg, or about 27 mg/kg. The fusion protein may be administered at an interval of about once every week, about once every two weeks, or about once every three weeks.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in anti-drug antibodies (ADA) in the subject after the first dose, after one treatment cycle, after two treatment cycles, after three treatment cycles, or even later. In some embodiments, the fusion protein is administered at a dose level from about 0.05 mg/kg to about 27 mg/kg, such as about 0.05 mg/kg, about 0.15 mg/kg, about 0.5 mg/kg, about 1 mg/kg, about 2.5 mg/kg, about 5 mg/kg, about 8 mg/kg, about 12 mg/kg, about 18 mg/kg, or about 27 mg/kg.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount does not result in anti-drug antibodies (ADA) in the subject.

In some embodiments, the present disclosure provides a method for treating a HER2-expressing tumor in a subject, wherein the method comprises administering a therapeutically effective amount of a provided HER2/4-1BB bispecific fusion protein comprising the amino acid sequences shown in SEQ ID NOs: 50 and 51, wherein the administered amount results in a response in the subject, such as a partial response, a complete response, and/or a sustained response (e.g., a sustained partial response or complete response) after cessation of the treatment.

In some embodiments, the subject has been treated with one or more cancer therapies before the treatment of a provided HER2/4-1BB bispecific fusion protein comprising the amino acid sequences shown in SEQ ID NOs: 50 and 51. In some embodiments, the subject has been treated before the treatment of the furoin protein with chemotherapy, a HER2-targeting drug such as trastuzumab or pertuzumab, a 4-1BB/4-1BBL pathway-targeting drug such as urelumab or utomilumab, a PD-1 signaling pathway-targeting drug such as nivolumab, pembrolizumab, or cemiplimab, a CTLA-4 signaling pathway-targeting drug such as ipilimumab, or combinations of any of the foregoing (e.g., a combination of chemotherapy and a HER2-targeting drug). In some embodiments, the subject the resistant to the one or more cancer therapies. In some embodiments, the subject has not been previously treated with a PD-1 signaling pathway-targeting drug. In some embodiments, the treatment with the HER2/4-1BB bispecific fusion protein does not comprise a (co-)treatment with a PD-1 signaling pathway-targeting drug. In some embodiments, the treatment with the HER2/4-1BB bispecific fusion protein provided herein comprises (co-)treatment with a PD-1 signaling pathway-targeting drug (or PD-1 axis inhibitor), such as an antibody specific for PD-1 (e.g., nivolumab, pembrolizumab, cemiplimab or tislelizumab) or PD-L1 (e.g., atezolizumab, avelumab, durvalumab or BMS-936559, preferably atezolizumab), e.g., as described in WO 2020/043683, which is herein incorporated by reference in its entirety.

In some embodiments, the subject administered with the provided fusion protein may have been previously treated with chemotherapy, a HER2-targeting drug or a combination thereof. In some particular embodiments, the subject may have been previously treated with a platinum, fluoropyrimidine and a HER2-targeting drug. Preferably, the HER2-targeting drug is an anti-HER2 antibody, wherein, more preferably, the anti-HER2 antibody is trastuzumab.

In some embodiments, the subject has been treated with a B cell depleting agent before the treatment of a provided HER2/4-1BB bispecific fusion protein comprising the amino acid sequences shown in SEQ ID NOs: 50 and 51. In some embodiments, the subject has been treated before the treatment of the furoin protein with an anti-CD20 antibody, such as rituximab, obinutuzumab, ocrelizumab, or veltuzumab. In some embodiments, the subject has been treated with obinutuzumab at a dose of about 1000 mg to about 2000 mg, about seven days before the treatment of the furoin protein. In some embodiments, the subject has been treated with obinutuzumab at a dose of about 2000 mg seven days before the treatment of the fusion protein or at a dose of 1000 mg seven days before and six days before the treatment of the fusion protein.

In some embodiments, the subject has less than about 1000, less than about 750, less than about 500, less than about 400, less than about 400, less than about 300, less than about 250, less than about 200, less than about 150, less than about 100, less than about 90, less than about 80, less than about 70, less than about 60, less than about 50, less than about 45, less than about 40, less than about 35, or even lower CD8+ T cells per mm² tumor tissue before the treatment of the treatment of a provided HER2/4-1BB bispecific fusion protein comprising the amino acid sequences shown in SEQ ID NOs: 50 and 51. In some embodiments, the subject has less than about 250 CD8+ T cells per mm² tumor tissue before the treatment of the treatment of a provided HER2/4-1BB bispecific fusion protein comprising the amino acid sequences shown in SEQ ID NOs: 50 and 51.

In some embodiments, a provided HER2/4-1BB bispecific fusion protein comprising the amino acid sequences shown in SEQ ID NOs: 50 and 51 may be administered to a subject who has a pre-treatment level of less than about 50%, less than about 40%, less than about 30%, less than about 25%, less than about 20%, less than about 15%, less than about 10%, less than about 5%, or even lower PD-L1+ cells of total immune cells. In some embodiments, a provided HER2/4-1BB bispecific fusion protein comprising the amino acid sequences shown in SEQ ID NOs: 50 and 51 may be administered to a subject who has a pre-treatment level of less than about 25% PD-L1+ cells of total immune cells.

In some embodiments, a provided HER2/4-1BB bispecific fusion protein comprising the amino acid sequences shown in SEQ ID NOs: 50 and 51 may be administered to a subject who has a pre-treatment level of less than about 250 CD8+ T cells per mm² tumor tissue and a pre-treatment level of less than about 25% PD-L1+ cells of total immune cells.

In some embodiments, the HER2-expressing tumor is a HER2-positive (HER2+) tumor. In some embodiments, the tumor is characterized by a HER2 status of IHC3+, IHC2+/(F)ISH+ or (F)ISH+, preferably IHC3+ or IHC2+/(F)ISH+. In some embodiments, the tumor exhibits HER2 gene amplification.

In some embodiments, the HER2-expressing tumor is characterized by a low expression of HER2. In some embodiments, the tumor is characterized by a HER2 status of IHC1+ or IHC2+/(F)ISH−. In some embodiments, the tumor does not exhibit HER2 gene amplification.

In some embodiments, the HER2/4-1BB comprising the amino acid sequences shown in SEQ ID NOs: 50 and 51 is administered at a dose level of about 0.0005 mg/kg, about 0.0015 mg/kg, about 0.005 mg/kg, about 0.015 mg/kg, about 0.05 mg/kg, about 0.15 mg/kg, about 0.5 mg/kg, about 1 mg/kg, about 2.5 mg/kg, about 5.0 mg/kg, about 8 mg/kg, about 12 mg/kg, about 18 mg/kg, about 27 mg/kg, or even higher. In some embodiments, the fusion protein is administered at a dose level of about 2.5 mg/kg, about 5.0 mg/kg, about 8 mg/kg, about 12 mg/kg, about 18 mg/kg, about 27 mg/kg, or even higher. In some embodiments, the fusion protein is administered at a dose level of about 8 mg/kg, about 12 mg/kg, about 18 mg/kg, about 27 mg/kg, or even higher.

In some embodiments, the HER2/4-1BB comprising the amino acid sequences shown in SEQ ID NOs: 50 and 51 is administered with a dosing schedule of about once every week, about once every two weeks, about once every three weeks, or about once every four weeks. In some embodiments, the fusion protein is administered with a dosing schedule of about twice a week, about once a week, about once every ten days, about once every two weeks, about once every three weeks, about once every four weeks, about once every five weeks, about once every month, about once every six weeks, about once every seven weeks, about once every eight weeks, or about once every two months.

In some embodiments, the HER2/4-1BB bispecific fusion protein comprising the amino acid sequences shown in SEQ ID NOs: 50 and 51 is administered to a subject at a dose of from about 2.5 mg/kg to about 27 mg/kg at an interval of about once every two weeks to about once every week, at a dose of from about 5 mg/kg to about 27 mg/kg at an interval of about once every two weeks to about once every week, at a dose of from about 8 mg/kg to about 27 mg/kg at an interval of about once every two weeks to about once every week, at a dose of from about 2.5 mg/kg to about 12 mg/kg at an interval of about once every two weeks to about once every week, at a dose of from about 5 mg/kg to about 12 mg/kg at an interval of about once every two weeks to about once every week, or at a dose of from about 8 mg/kg to about 18 mg/kg at an interval of about once every two weeks to about once every week.

In some embodiments, the HER2/4-1BB bispecific fusion protein comprising the amino acid sequences shown in SEQ ID NOs: 50 and 51 is administered to a subject at a dose of about 2.5 mg/kg at an interval of about once every two weeks to about once every week, at a dose of about 5 mg/kg at an interval of about once every two weeks to about once every week, at a dose of about 8 mg/kg at an interval of about once every two weeks to about once every week, at a dose of about 12 mg/kg at an interval of about once every two weeks to about once every week, at a dose of about 18 mg/kg at an interval of about once every two weeks to about once every week, or at a dose of about 27 mg/kg at an interval of about once every two weeks to about once every week.

In some embodiments, the HER2/4-1BB bispecific fusion protein comprising the amino acid sequences shown in SEQ ID NOs: 50 and 51 is administered to a subject at a dose of from about 2.5 mg/kg to about 27 mg/kg at an interval of about once every two weeks, at a dose of from about 5 mg/kg to about 27 mg/kg at an interval of about once every two weeks, at a dose of from about 8 mg/kg to about 27 mg/kg at an interval of about once every two weeks, at a dose of from about 2.5 mg/kg to about 12 mg/kg at an interval of about once every two weeks, at a dose of from about 5 mg/kg to about 12 mg/kg at an interval of about once every two weeks, or at a dose of from about 8 mg/kg to about 18 mg/kg at an interval of about once every two weeks.

In some embodiments, the HER2/4-1BB bispecific fusion protein comprising the amino acid sequences shown in SEQ ID NOs: 50 and 51 is administered to a subject at a dose of about 2.5 mg/kg, about 5 mg/kg, about 8 mg/kg, about 12 mg/kg, about 18 mg/kg, or about 27 mg/kg at an interval of about once every two weeks.

In some embodiments, the HER2/4-1BB bispecific fusion protein comprising the amino acid sequences shown in SEQ ID NOs: 50 and 51 is administered to a subject at a dose of from about 2.5 mg/kg to about 27 mg/kg at an interval of about once every week, at a dose of from about 5 mg/kg to about 27 mg/kg at an interval of about once every week, at a dose of from about 8 mg/kg to about 27 mg/kg at an interval of about once every week, at a dose of from about 2.5 mg/kg to about 12 mg/kg at an interval of about once every week, at a dose of from about 5 mg/kg to about 12 mg/kg at an interval of about once every week, or at a dose of from about 8 mg/kg to about 18 mg/kg at an interval of about once every week.

In some embodiments, the HER2/4-1BB bispecific fusion protein comprising the amino acid sequences shown in SEQ ID NOs: 50 and 51 is administered to a subject at a dose of about 2.5 mg/kg, about 5 mg/kg, about 8 mg/kg, about 12 mg/kg, about 18 mg/kg, or about 27 mg/kg at an interval of about once every week.

In some embodiments, the HER2/4-1BB bispecific fusion protein provided herein, e.g., comprising the amino acid sequences shown in SEQ ID NOs: 50 and 51, is administered to a subject at a first dose and, subsequently, at a second dose, wherein the first dose exceeds the second dose. In some embodiments, the fusion protein is administered at the first dose up to five times, up to four times, up to three times or up to two times. In some embodiments, the fusion protein is administered two times at the first dose.

In some embodiments, the fusion protein is administered at an interval of about once every three weeks, about once every two weeks, or about once every week. In some embodiments, the fusion protein is administered at an interval of about once every week. In some embodiments, the fusion protein is administered at an interval of about once every two weeks. In some embodiments, the fusion protein is administered at an interval of about once every three weeks.

In some embodiments, the first dose is from about 5 mg/kg to about 27 mg/kg. In some embodiments, the first dose is from about 12 mg/kg to about 27 mg/kg. In some embodiments, the first dose is about 18 mg/kg. In some embodiments, the first dose is about 12 mg/kg.

In some embodiments, the second dose is from about 2.5 mg/kg to about 18 mg/kg. In some embodiments, the second dose is from about 2.5 mg/kg to about 12 mg/kg. In some embodiments, the second dose is about 8 mg/kg. In some embodiments, the second dose is about 5 mg/kg. In some embodiments, the second dose is about 2.5 mg/kg.

In some embodiments, the first dose may be about 18 mg/kg, and the second dose may be about 8 mg/kg. In some embodiments, the first dose may be about 18 mg/kg, and the second dose may be about 5 mg/kg. In some embodiments, the first dose may be about 18 mg/kg, and the second dose may be about 2.5 mg/kg. In some embodiments, the first dose may be about 12 mg/kg, and the second dose may be about 8 mg/kg. In some embodiments, the first dose may be about 12 mg/kg, and the second dose may be about 5 mg/kg. In some embodiments, the first dose may be about 12 mg/kg, and the second dose may be about 2.5 mg/kg.

In some embodiments, the HER2/4-1BB bispecific fusion protein comprising the amino acid sequences shown in SEQ ID NOs: 50 and 51 is administered to a subject by infusion. In some embodiments, the HER2/4-1BB bispecific fusion protein comprising the amino acid sequences shown in SEQ ID NOs: 50 and 51 is administered to a subject by intravenous infusion.

In some embodiments, methods provided by the present disclosure may further comprise an additional therapy. In some embodiments, the additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. Such additional therapy may be in the form of adjuvant or neoadjuvant therapy. In some embodiments, an additional therapy is the administration of a small molecule enzymatic inhibitor or an anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is the administration of agents that reduce anti-drug antibodies (ADAs). In some embodiments, the additional therapy is the administration of B cell depletion agents. In some embodiments, treatment with the HER2/4-1BB bispecific fusion protein comprises administering to the subject at least one additional anti-tumor drug. In some embodiments, treatment with the HER2/4-1BB bispecific fusion protein comprises administering to the subject a chemotherapeutic drug (e.g., a taxane, such as paclitaxel), an anti-angiogenic drug, or a combination of both. In some embodiments, the anti-angiogenic drug or agent is an inhibitor of the VEGF-VEGFR pathway, such as an antibody, an antigen-binding fragment thereof, or another binding agent specific for a member of the VEGF-VEGFR pathway (e.g., VEGF-A or VEGFR-2), or an anti-angiogenic small molecule, such as regorafenib (which may be used for the treatment of colorectal cancer, for example). Suitable inhibitors of the VEGF-VEGFR pathway include, but are not limited to, the anti-VEGFR-2 antibody ramucirumab, the anti-VEGF-A antibody bevacizumab and the fusion protein aflibercept. In some embodiments, the anti-angiogenic drug is ramucirumab.

### C. Pharmaceutical Formulations

In some embodiments, a provided HER2/4-1BB bispecific fusion protein comprising the amino acid sequences shown in SEQ ID NOs: 50 and 51 may be formulated in accordance with standard pharmaceutical practice for use as “active ingredients” of therapeutic compositions. Compositions comprising such molecules may contain one or more pharmaceutically acceptable carrier, glidant, diluent, or excipient, which facilitate administration of the composition and/or facilitate delivery of the composition to the site of action. Suitable carriers, diluents and excipients are known to those skilled in the art and include materials such as carbohydrates, waxes, water soluble and/or swellable polymers, hydrophilic or hydrophobic materials, gelatin, oils, solvents, water and the like. Compositions of the disclosure may be in any suitable form, for example tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders, to name just a few non-limiting alternatives. Such compositions (or formulations) may be prepared using methods known in the art, such as conventional dissolution and mixing procedures.

In some embodiments, formulations of the disclosure may be prepared for various routes and types of administration in the form of a lyophilized formulation, milled powder, or an aqueous solution. In some embodiments, formulations of the disclosure may be prepared for intravenous infusion.

In some embodiments, a provided HER2/4-1BB bispecific fusion protein comprising the amino acid sequences shown in SEQ ID NOs: 50 and 51 may be formulated as aqueous solution with a target protein concentration of about 25 mg/mL in 20 mM Histidine, 250 mM Sorbitol, pH 6.3, 0.01% PS80.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art and that such modifications and variations are considered to be within the scope of this disclosure.

The invention may further be characterized by following items:

Item 1. A fusion protein for use in treating a HER2-expressing tumor in a subject, wherein the treatment comprises administering the fusion protein at a first dose and, subsequently, at a second dose, wherein the first dose exceeds the second dose, wherein the fusion protein comprises an antibody specific for HER2 fused at the C-terminus of both heavy chains to the N-terminus of a lipocalin mutein specific for 4-1BB, wherein the antibody comprises: i. three heavy chain complementarity-determining regions (CDRs) shown in SEQ ID NO: 40, SEQ ID NO: 41, and SEQ ID NO: 42, and three light chain CDRs shown in SEQ ID NO: 43, SEQ ID NO: 44, and SEQ ID NO: 45; and ii. a heavy chain with at least 95% sequence identity to the amino acid sequence shown in SEQ ID NO: 49, and a light chain with at least 95% sequence identity to the amino acid sequence shown in SEQ ID NO: 50; and wherein the lipocalin mutein has at least 95% sequence identity to an amino acid sequence shown in SEQ ID NO: 22.

Item 2. The fusion protein for the use of item 1, wherein the fusion protein is administered at the first dose up to five times, up to four times, up to three times or up to two times.

Item 3. The fusion protein for the use of item 1 or 2, wherein the fusion protein is administered two times at the first dose.

Item 4. The fusion protein for the use of any one of items 1-3, wherein the first dose is from about 5 mg/kg to about 27 mg/kg.

Item 5. The fusion protein for the use of any one of items 1-4, wherein the first dose is from about 12 mg/kg to about 27 mg/kg.

Item 6. The fusion protein for the use of any one of items 1-5, wherein the first dose is about 18 mg/kg.

Item 7. The fusion protein for the use of any one of items 1-5, wherein the first dose is about 12 mg/kg.

Item 8. The fusion protein for the use of any one of items 1-7, wherein the second dose is from about 2.5 mg/kg to about 18 mg/kg.

Item 9. The fusion protein for the use of any one of items 1-8, wherein the second dose is from about 2.5 mg/kg to about 12 mg/kg.

Item 10. The fusion protein for the use of any one of items 1-9, wherein the second dose is about 8 mg/kg.

Item 11. The fusion protein for the use of any one of items 1-9, wherein the second dose is about 5 mg/kg.

Item 12. The fusion protein for the use of any one of items 1-9, wherein the second dose is about 2.5 mg/kg.

Item 13. The fusion protein for the use of any one of items 1-12, wherein the treatment comprises administering the fusion protein at an interval of about once every three weeks, about once every two weeks, or about once every week.

Item 14. The fusion protein for the use of any one of items 1-13, wherein the treatment comprises administering the fusion protein at an interval of about once every week.

Item 15. The fusion protein for the use of any one of items 1-13, wherein the treatment comprises administering the fusion protein at an interval of about once every two weeks.

Item 16. The fusion protein for the use of any one of items 1-13, wherein the treatment comprises administering the fusion protein at an interval of about once every three weeks.

Item 17. The fusion protein for the use of any one of items 1-5, 8, 9 and 13, comprising administering the fusion protein two times at a dose of about 18 mg/kg and, subsequently, at a dose of about 8 mg/kg, wherein the fusion protein is administered at an interval of about once every week.

Item 18. The fusion protein for the use of any one of items 1-5, 8, 9 and 13, comprising administering the fusion protein two times at a dose of about 18 mg/kg and, subsequently, at a dose of about 5 mg/kg, wherein the fusion protein is administered at an interval of about once every week.

Item 19. The fusion protein for the use of any one of items 1-5, 8, 9 and 13, comprising administering the fusion protein two times at a dose of about 18 mg/kg and, subsequently, at a dose of about 2.5 mg/kg, wherein the fusion protein is administered at an interval of about once every week.

Item 20. The fusion protein for the use of any one of items 1-5, 8, 9 and 13, comprising administering the fusion protein two times at a dose of about 12 mg/kg and, subsequently, at a dose of about 8 mg/kg, wherein the fusion protein is administered at an interval of about once every week.

Item 21. The fusion protein for the use of any one of items 1-5, 8, 9 and 13, comprising administering the fusion protein two times at a dose of about 12 mg/kg and, subsequently, at a dose of about 5 mg/kg, wherein the fusion protein is administered at an interval of about once every week.

Item 22. The fusion protein for the use of any one of items 1-5, 8, 9 and 13, comprising administering the fusion protein two times at a dose of about 12 mg/kg and, subsequently, at a dose of about 2.5 mg/kg, wherein the fusion protein is administered at an interval of about once every week.

Item 23. The fusion protein for the use of any one of items 1-5, 8, 9 and 13, comprising administering the fusion protein two times at a dose of about 18 mg/kg and, subsequently, at a dose of about 8 mg/kg, wherein the fusion protein is administered at an interval of about once every two weeks.

Item 24. The fusion protein for the use of any one of items 1-5, 8, 9 and 13, comprising administering the fusion protein two times at a dose of about 18 mg/kg and, subsequently, at a dose of about 5 mg/kg, wherein the fusion protein is administered at an interval of about once every two weeks.

Item 25. The fusion protein for the use of any one of items 1-5, 8, 9 and 13, comprising administering the fusion protein two times at a dose of about 18 mg/kg and, subsequently, at a dose of about 2.5 mg/kg, wherein the fusion protein is administered at an interval of about once every two weeks.

Item 26. The fusion protein for the use of any one of items 1-5, 8, 9 and 13, comprising administering the fusion protein two times at a dose of about 12 mg/kg and, subsequently, at a dose of about 8 mg/kg, wherein the fusion protein is administered at an interval of about once every two weeks.

Item 27. The fusion protein for the use of any one of items 1-5, 8, 9 and 13, comprising administering the fusion protein two times at a dose of about 12 mg/kg and, subsequently, at a dose of about 5 mg/kg, wherein the fusion protein is administered at an interval of about once every two weeks.

Item 28. The fusion protein for the use of any one of items 1-5, 8, 9 and 13, comprising administering the fusion protein two times at a dose of about 12 mg/kg and, subsequently, at a dose of about 2.5 mg/kg, wherein the fusion protein is administered at an interval of about once every two weeks.

Item 29. The fusion protein for the use of any one of items 1-5, 8, 9 and 13, comprising administering the fusion protein two times at a dose of about 18 mg/kg and, subsequently, at a dose of about 8 mg/kg, wherein the fusion protein is administered at an interval of about once every three weeks.

Item 30. The fusion protein for the use of any one of items 1-5, 8, 9 and 13, comprising administering the fusion protein two times at a dose of about 18 mg/kg and, subsequently, at a dose of about 5 mg/kg, wherein the fusion protein is administered at an interval of about once every three weeks.

Item 31. The fusion protein for the use of any one of items 1-5, 8, 9 and 13, comprising administering the fusion protein two times at a dose of about 18 mg/kg and, subsequently, at a dose of about 2.5 mg/kg, wherein the fusion protein is administered at an interval of about once every three weeks.

Item 32. The fusion protein for the use of any one of items 1-5, 8, 9 and 13, comprising administering the fusion protein two times at a dose of about 12 mg/kg and, subsequently, at a dose of about 8 mg/kg, wherein the fusion protein is administered at an interval of about once every three weeks.

Item 33. The fusion protein for the use of any one of items 1-5, 8, 9 and 13, comprising administering the fusion protein two times at a dose of about 12 mg/kg and, subsequently, at a dose of about 5 mg/kg, wherein the fusion protein is administered at an interval of about once every three weeks.

Item 34. The fusion protein for the use of any one of items 1-5, 8, 9 and 13, comprising administering the fusion protein two times at a dose of about 12 mg/kg and, subsequently, at a dose of about 2.5 mg/kg, wherein the fusion protein is administered at an interval of about once every three weeks.

Item 35. The fusion protein for the use of any one of items 1-34, wherein the fusion protein has at least about 95% sequence identity to the amino acid sequences shown in SEQ ID NOs: 50 and 51.

Item 36. The fusion protein for the use of any one of items 1-35, wherein the fusion protein comprises the amino acid sequences shown in SEQ ID NO: 50 and 51.

Item 37. The fusion protein for the use of any one of items 1-36, wherein the fusion protein comprises two chains having the amino acid sequence shown in SEQ ID NO: 50 and two chains having the amino acid sequence shown in SEQ ID NO: 51.

Item 38. A fusion protein for use in treating a HER2-expressing tumor in a subject, wherein the treatment comprises administering the fusion protein two times at a dose of about 18 mg/kg and, subsequently, at a dose of about 8 mg/kg, wherein the fusion protein is administered at an interval of about once every two weeks, wherein the fusion protein comprises two chains having the amino acid sequence shown in SEQ ID NO: 50 and two chains having the amino acid sequence shown in SEQ ID NO: 51.

Item 39. The fusion protein for the use of any one of items 1-38, wherein the treatment is associated with: a. an at least about 1.5-fold increase of CD8+ T cell numbers in the full tumor tissue; b. an at least about 1.5-fold increase of CD8+ T cell numbers in tumor cells; c. an at least about 1.5-fold increase of CD8+Ki67+ T cell numbers in the full tumor tissue; d. an at least about 1.5-fold increase of CD8+Ki67+ T cell numbers in tumor cells; e. an increase of CD8+ T cells from a pre-treatment level of less than about 500 per mm2 of a measured area, wherein the measured area is an area of the full tumor tissue, tumor stroma, or tumor cells; f. an increase of the level of soluble 4-1BB (s4-1BB) in the blood serum; g. an at least 30% decrease in the target lesion; h. stable disease; i. a partial response; or j. a complete response.

Item 40. The fusion protein for the use of any one of items 1-39, wherein the tumor is selected from the group consisting of gastric cancer, gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer, colon cancer, pancreatic cancer, biliary tract cancer, salivary duct cancer, bladder cancer, and cancer of unknown primary.

Item 41. The fusion protein for the use of any one of items 1-39, wherein the tumor is gastric cancer.

Item 42. The fusion protein for the use of any one of items 1-39, wherein the tumor is gastroesophageal cancer, preferably gastroesophageal junction cancer.

Item 43. The fusion protein for the use of any one of items 1-42, wherein the tumor is gastric or gastroesophageal junction adenocarcinoma.

Item 44. The fusion protein for the use of any one of items 1-39, wherein the tumor is colorectal cancer.

Item 45. The fusion protein for the use of any one of items 1-39, wherein the tumor is lung cancer, preferably non-small cell lung cancer.

Item 46. The fusion protein for the use of any one of items 1-45, wherein the subject has (i) a pre-treatment level of less than about 250 CD8+ T cells per mm2 of a measured area, wherein the measured area is an area of the full tumor tissue, tumor stroma, or tumor cells, and (ii) a pre-treatment level of less than about 25% PD-L1+ cells of total immune cells.

Item 47. The fusion protein for the use of any one of items 1-46, wherein the tumor is a HER2-positive (HER2+) tumor.

Item 48. The fusion protein for the use of any one of items 1-47, wherein the tumor is characterized by a HER2 status of IHC3+, IHC2+/(F)ISH+ or (F)ISH+, preferably IHC3+ or IHC2+/(F)ISH+.

Item 49. The fusion protein for the use of item 47 or 48, wherein the tumor exhibits HER2 gene amplification.

Item 50. The fusion protein for the use of any one of items 1-46, wherein the tumor is characterized by a low expression of HER2.

Item 51. The fusion protein for the use of any one of items 1-46 and 50, wherein the tumor is characterized by a HER2 status of IHC1+ or IHC2+/(F)ISH−.

Item 52. The fusion protein for the use of item 50 or 51, wherein the tumor does not exhibit HER2 gene amplification.

Item 53. The fusion protein for the use of any one of items 1-52, wherein the treatment further comprises administering a chemotherapeutic drug, an anti-angiogenic drug, or a combination of both.

Item 54. A fusion protein for use in treating a tumor in a subject, wherein the tumor is characterized by a low expression of HER2, wherein the treatment comprises administering the fusion protein at a dose of from about 2.5 mg/kg to about 27 mg/kg, wherein the fusion protein comprises an antibody specific for HER2 fused at the C-terminus of both heavy chains to the N-terminus of a lipocalin mutein specific for 4-1BB, wherein the antibody comprises: i. three heavy chain complementarity-determining regions (CDRs) shown in SEQ ID NO: 40, SEQ ID NO: 41, and SEQ ID NO: 42, and three light chain CDRs shown in SEQ ID NO: 43, SEQ ID NO: 44, and SEQ ID NO: 45; and ii. a heavy chain with at least 95% sequence identity to the amino acid sequence shown in SEQ ID NO: 49, and a light chain with at least 95% sequence identity to the amino acid sequence shown in SEQ ID NO: 50; and wherein the lipocalin mutein has at least 95% sequence identity to an amino acid sequence shown in SEQ ID NO: 22.

Item 55. The fusion protein for the use of item 54, wherein the tumor is characterized by a HER2 status of IHC1+ or IHC2+/(F)ISH−.

Item 56. The fusion protein for the use of item 54 or 55, wherein the tumor does not exhibit HER2 gene amplification.

Item 57. The fusion protein for the use of any one of items 54-56, wherein the fusion protein is administered at an interval of about once every three weeks, about once every two weeks, or about once every week.

Item 58. The fusion protein for the use of any one of items 54-57, wherein the fusion protein is administered at a dose of about 2.5 mg/kg.

Item 59. The fusion protein for the use of any one of items 54-57, wherein the fusion protein is administered at a dose of about 5 mg/kg.

Item 60. The fusion protein for the use of any one of items 54-57, wherein the fusion protein is administered at a dose of about 8 mg/kg.

Item 61. The fusion protein for the use of any one of items 54-57, wherein the fusion protein is administered at a dose of about 12 mg/kg.

Item 62. The fusion protein for the use of any one of items 54-57, wherein the fusion protein is administered at a dose of about 18 mg/kg.

Item 63. The fusion protein for the use of any one of items 54-62, wherein the fusion protein has at least about 95% sequence identity to the amino acid sequences shown in SEQ ID NOs: 50 and 51.

Item 64. The fusion protein for the use of any one of items 54-63, wherein the fusion protein comprises the amino acid sequences shown in SEQ ID NO: 50 and 51.

Item 65. The fusion protein for the use of any one of items 54-64, wherein the fusion protein comprises two chains having the amino acid sequence shown in SEQ ID NO: 50 and two chains having the amino acid sequence shown in SEQ ID NO: 51.

Item 66. The fusion protein for the use of any one of items 54-65, wherein the treatment is associated with: a. an at least about 1.5-fold increase of CD8+ T cell numbers in the full tumor tissue; b. an at least about 1.5-fold increase of CD8+ T cell numbers in tumor cells; c. an at least about 1.5-fold increase of CD8+Ki67+ T cell numbers in the full tumor tissue; d. an at least about 1.5-fold increase of CD8+Ki67+ T cell numbers in tumor cells; e. an increase of CD8+ T cells from a pre-treatment level of less than about 500 per mm2 of a measured area, wherein the measured area is an area of the full tumor tissue, tumor stroma, or tumor cells; f. an increase of the level of soluble 4-1BB (s4-1BB) in the blood serum; g. an at least 30% decrease in the target lesion; h. stable disease; i. a partial response; or j. a complete response.

Item 67. The fusion protein for the use of any one of items 54-66, wherein the tumor is selected from the group consisting of gastric cancer, gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer, colon cancer, pancreatic cancer, biliary tract cancer, salivary duct cancer, bladder cancer, and cancer of unknown primary.

### VI. EXAMPLES

### Example 1: T Cell Immunogenicity Assessment of HER2/4-1BB Bispecific Fusion Proteins

To investigate the risk of the formation of anti-drug antibodies in man, an in-vitro T cell immunogenicity assessment was performed for the HER2/4-1 BB bispecific fusion proteins SEQ ID NOs: 50 and 51, SEQ ID NOs: 50 and 53, SEQ ID NOs: 52 and 49 and SEQ ID NOs: 54 and 49, as well as for reference antibody SEQ ID NOs: 50 and 48.

Human peripheral blood mononuclear cells (PBMCs) from 32 donors, selected to cover human leukocyte antigen (HLA) allotypes and reflective of the distribution in a global population, were thawed, washed, and seeded onto 96-well plates at a density of 3×10⁵ cells per well. Test articles, diluted in assay media, were added to the cells at a concentration of 30 μg/mL and then incubated for 7 days in a humidified atmosphere at 37° C. and 5% CO₂. Assay medium alone was used as a blank, and keyhole limpet hemocyanine (KLH) was tested as a naïve positive control. On day 7, PBMCs were labelled for surface phenotypic CD3+ and CD4+ markers and for DNA-incorporated EdU (5-ethynyl-2′deoxyuridine), used as a cell proliferation marker. The percentage of CD3+CD4+EdU+ proliferating cells was measured using a Guava easyCyte 8HT flow cytometer and analyzed using GuavaSoft InCyte software.

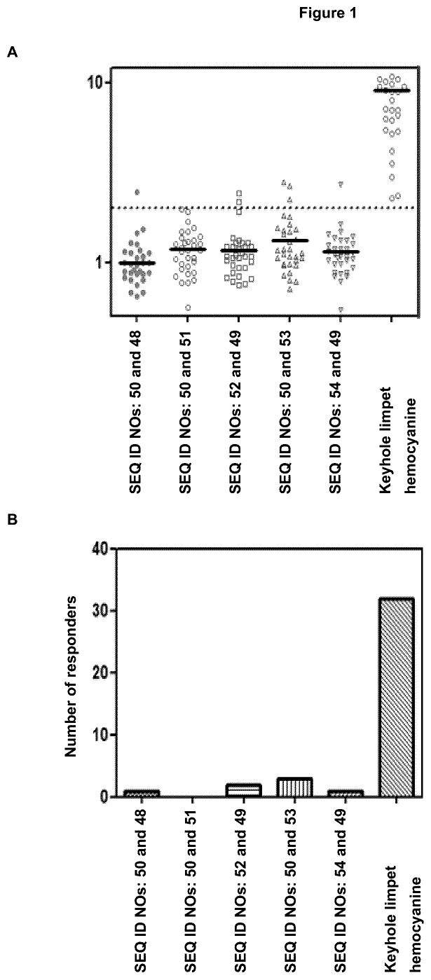

Results of this assay are shown in FIG. 1. In FIG. 1A, the stimulation index was plotted, which was obtained by the ratio of proliferation in the presence vs. absence of test article. The threshold that defines a responding donor (stimulation index >2) is indicated as a dotted line. In FIG. 1B, the number of responding donors as defined by this threshold was plotted. Evidently, the number of donors responding to the reference antibody SEQ ID NOs: 50 and 48 lies at one and is therefore small, while all 32 donors respond to the positive control KLH with strong proliferation above the threshold. For the bispecific fusion proteins, the number of responding donors are zero, one, two, and three for SEQ ID NOs: 50 and 51, SEQ ID NOs: 54 and 49, SEQ ID NOs: 50 and 53, and SEQ ID NOs: 52 and 49, respectively.

The results demonstrate that the bispecific fusion proteins, in particular SEQ ID NOs: 50 and 51 and SEQ ID NOs: 54 and 49, induce little response in the in-vitro T cell immunogenicity assessment, indicating low risk of inducing immunogenic responses in man.

### Example 2: In-Vitro T Cell Activation of PRS-343

HER2 target-dependent T cell activation mediated by PRS-343 was assessed in co-culture experiments using a panel of cell lines expressing different levels of HER2. Cancer cell lines representing a range of clinically relevant levels of HER2 receptor (NCI-N87: HER2 high, MKN45: HER2 low, HepG2: HER2 null) were tested for their ability to mediate clustering of PRS-343 and subsequent activation of T cells. To evaluate a potential therapeutic window, cell lines derived from healthy tissues known to express background levels of HER2 were also included.

Briefly, cancer cells or cells derived from healthy tissue pretreated with 10 μg/mL of mitomycin C (Sigma Aldrich) were seeded in culture plates pre-coated with anti-CD3 and incubated overnight at 37° C. in a humidified 5% CO₂ atmosphere. T cell suspension (5×10⁴ cells) together with test article was added and incubated for 3 days. The level of T cell activation was measured by quantifying of human IL-2 in the supernatant, using an electrochemiluminescence (ECL) immunoassay (using IL2 DuoSet kit; R&D Systems).

Specific activation of the 4-1BB pathway by PRS-343 was also assessed using a luciferase reporter cell assay (Promega), where a 4-1BB overexpressing reporter cell line (NF-κB-Luc2/4-1BB Jurkat cells) was cocultured with HER2-positive tumor cell lines and where 4-1BB pathway activation was measured by luminescence.

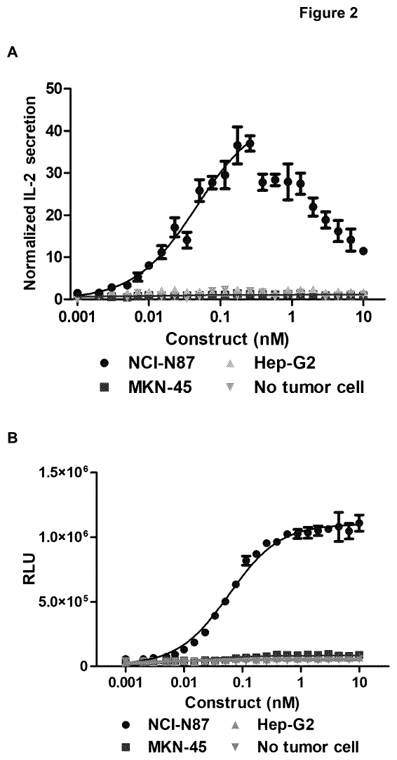

Results of an exemplary experiments are shown in FIG. 2. In the presence of HER2-positive cell lines, a dose-dependent induction of IL-2 was observed with PRS-343. Particularly, PRS-343 induces IL-2 production in the presence of HER2-positive NCI-N87 cells with a potency of about 35 pmol/L (EC₅₀). When the experiment was performed with cell lines expressing basal levels of HER2, no PRS-343-dependent IL-2 induction was observed. Additionally, PRS-343 induces 4-1BB clustering and downstream signaling in a Jurkat NF-κB reporter cell line in the presence of HER2-positive cells with a potency of approximately 50 pmol/L (EC₅₀).

A bell-shaped response was observed for both in the primary T cell activation assay and the Jurkat NF-κB reporter assay, suggesting the response requires the formation of a ternary complex of the tumor cell target HER2, the drug PRS-343, and the T cell receptor 4-1BB and can be disrupted when HER2 and 4-1BB are individually saturated with PRS-343.

### Example 3: Dose Escalation Study of PRS-343 in Patients with HER2+ Advanced or Metastatic Solid Tumors

Example 3 provides information on this study for Cohorts 1-11, with additional information for Cohorts 1-13 provided in Example 4.

### A. Study Objectives and Overview

This example describes a Phase 1, open-Label, dose escalation study of PRS-343 in patients with HER2+ advanced or metastatic solid tumors for which standard treatment options are not available, are no longer effective, are not tolerated, or the patient has refused standard therapy. The primary objective of the study is to characterize the safety profile and identify the maximum tolerated dose (MTD) or recommended Phase 2 dose (RP2D) of PRS-343. The secondary objective of the study is to characterize the pharmacokinetic (PK) profile of PRS-343, investigate dosing schedule(s) of PRS-343, obtain preliminary estimates of efficacy of PRS-343, assess the potential immunogenicity of PRS-343, assess the pharmacodynamic (PD) effects of PRS-343, and assess possible PK/safety, PK/PD and PK/efficacy correlations.

PRS-343 was supplied as an aqueous solution in 20 mL glass vials containing 16 mL of PRS-343 drug product at a target protein concentration of 25 mg/mL in 20 mM Histidine, 250 mM Sorbitol, pH 6.3, 0.01% PS80. Enrolled subjects received PRS-343 administered by intravenous (IV) infusion over 2 hours, every 3 weeks (Q3W, 21-day cycles) (Schedule 1) initially. If safety, PK, and PD data suggested a different dosing schedule should be evaluated, Schedule 2 or 3 (dosing every 2 weeks (Q2W) or every 4 weeks (Q4W) in a 28-day cycle, respectively) might be conducted. Separate MTDs might be determined for each schedule evaluated. Patients were allocated to different dose levels in dedicated cohorts (Table 1) and received PRS-343 on Day 1 of each 21-day cycle per Schedule 1, on Days 1 and 15 of each 28-day cycle per Schedule 2, or on Day 1 of each 28-day cycle per Schedule 3.

TABLE 1
PRS-343 dose levels

| Cohort | Dose (mg/kg) |
|---|---|
| 1 | 0.0005 |
| 2 | 0.0015 |
| 3 | 0.005 |
| 4 | 0.015 |
| 5 | 0.05 |
| 6 | 0.15 |
| 7 | 0.5 |
| 8 | 1.0 |
| 9 | 2.5 |
| 10 | 5.0 |
| 11 | 8.0 |

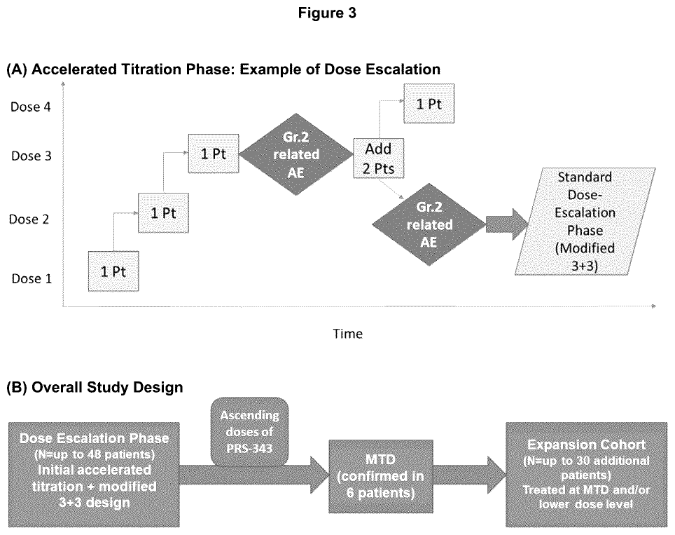

An accelerated titration design was utilized for the initial cohorts (FIG. 3A). Only 1 patient per cohort was enrolled in each escalating dose cohort until a patient experiences a Grade 2 treatment related adverse effect (AE) in Cycle 1, at which time 2 additional patients were enrolled. If a second patient experienced a Grade 2 treatment-related AE, the standard dose-escalation phase was initiated. If neither patient experienced a Grade 2 treatment-related AE, the accelerated titration continued. If a single patient experienced a dose-limiting toxicity (DLT), the modified 3+3 design was initiated (FIG. 3B). In the standard dose-escalation phase, a modified 3+3 design was utilized, allowing 3 or 4 patients to be enrolled in a cohort with expansion up to a total of 6 evaluable patients if a DLT is observed. The modified 3+3 design was scheduled to be initiated for dose levels 8 through 11 and higher (1 mg/kg to 8 mg/kg or higher respectively) if not initiated previously. After each cohort has been enrolled and all patients in the cohort have completed Cycle 1, safety data from all cohorts were reviewed to determine whether to proceed with further dose escalation.

Following identification of a non-tolerated dose, enrollment at the preceding dose would resume until that dose has been administered to 6 evaluable patients. An MTD is defined as the dose level below the dose inducing DLT in 33% of patients. At least 6 evaluable patients must be evaluated in the dose level for it to be called the MTD. Upon establishing MTD, up to 30 additional patients are enrolled in individual expansion cohorts at the MTD and/or at a lower dose level if safety/PD/PK/efficacy data support further evaluation of a lower dose level in order to determine the RP2D.

Subjects were enrolled in the study based on the following criteria: 1. Signed written informed consent obtained prior to performing any study procedure, including screening procedures; 2. Men and women ≥18 years; 3. Dose escalation: histologically or cytologically confirmed diagnosis of unresectable/locally advanced and/or metastatic HER2+ solid tumor malignancy and for which the standard therapies are not available, are no longer effective, are not tolerated, or have been declined by the patient. Expansion cohort: unresectable/locally advanced or metastatic HER2+ solid tumors considered likely to respond to a HER2-targeted 4-1BB agonist (e.g. gastric/gastroesophageal/esophageal, breast, bladder); 4. Dose escalation and expansion cohort: HER2+ solid tumors documented by clinical pathology report; 5. Patients with breast cancer and gastric and gastroesophageal junction cancer must have received at least 1 prior HER2 targeted therapy for advanced/metastatic disease; 6. Eastern Cooperative Oncology Group (ECOG) performance status (PS) 0-1; 7. Estimated life expectancy of at least 3 months; 8. Dose Escalation: evaluable or measurable disease according to RECIST v1.1. Expansion Cohort (additional 30 patients): measurable disease according to RECIST; 9. Adequate organ function as defined below: a) serum AST and ALT≤3×ULN; if liver meets present ≤5×ULN. b) total serum bilirubin ≤1.5×ULN. C) serum creatinine ≤1.5×ULN OR calculated glomerular filtration rate (GFR) by Cockcroft-Gault formula ≥50 mL/min. d) Hemoglobin ≥9 g/dL. e) ANC≥1500/mm³. f) Platelet count ≥75,000/mm³. g) Left ventricular ejection fraction (LVEF) determined by echocardiogram or multigated acquisition scan ≥50%; 10. Any prior cumulative doxorubicin dose must be ≤360 mg/m²; prior cumulative epirubicin dose must be ≤720 mg/m²; 11. Women of childbearing potential must have a negative serum or urine pregnancy test within 96 hours prior to start of study drug; 12. Women must not be breastfeeding; 13. Women of childbearing potential must agree to follow instruction for method(s) of contraception for the duration of treatment with study drug PRS-343 plus 90 days post-treatment completion; 14. Males who are sexually active with women of childbearing potential must agree to follow instructions for method(s) of contraception for the duration of treatment with study drug PRS-343 plus 90 days post-treatment completion.

Additionally, subjects who met any of the following criteria were not enrolled: 1. Known uncontrolled central nervous system (CNS) metastases and/or carcinomatous meningitis. Note: Patients with previously treated brain metastases may participate provided they are stable (without evidence of progression by imaging for at least 4 weeks prior to the first dose of study treatment and any neurologic symptoms have returned to baseline), have no evidence of new or enlarging brain metastases, and are clinically stable off steroids for at least 7 days prior to study treatment. Carcinomatous meningitis precludes a patient from study participation regardless of clinical stability; 2. History of acute coronary syndromes, including myocardial infarction, coronary artery bypass graft, unstable angina, coronary angioplasty or stenting within past 24 weeks; 3. History of or current Class II, III or IV heart failure as defined by the New York Heart Association (NYHA) functional classification system; 4. History of ejection fraction drop below the lower limit of normal with trastuzumab and/or pertuzumab; 5. Medical, psychiatric, cognitive or other conditions that compromise the patient's ability to understand the patient information, to give informed consent, to comply with the study protocol or to complete the study; 6. Any severe concurrent disease or condition (includes active infections, cardiac arrhythmia, interstitial lung disease) that in the judgment of the investigator would make study participation inappropriate for the patient; 7. Previously known active infection with human immunodeficiency virus (HIV); or hepatitis B or hepatitis C infection. Patients with positive hepatitis B core antibody (HBcAb) require assessment and monitoring of virus deoxyribonucleic acid (DNA) status; patients with positive hepatitis C virus (HCV) core antibody can enroll if HCV ribonucleic acid (RNA) is negative; 8. History of infusion reactions to any component/excipient of PRS-343; 9. Systemic steroid therapy (>10 mg daily prednisone or equivalent) or any other form of immunosuppressive therapy within 7 days prior to the first dose of study treatment (Note: topical, inhaled, nasal and ophthalmic steroids are not prohibited); 10. Autoimmune disease that has required systemic treatment in the past (i.e., with use of disease-modifying agents, corticosteroids, or immunosuppressive drugs). Replacement therapy (e.g., thyroxine, insulin, or physiologic corticosteroid replacement therapy for adrenal or pituitary insufficiency, etc.) is allowed; 11. Has not recovered from the adverse effect of previous anticancer treatments to pretreatment baseline or Grade 1 except for alopecia, anemia (hemoglobin levels must meet the study inclusion criteria) and peripheral neuropathy (which must have recovered to s Grade 2) nausea and diarrhea if anti-emetic and anti-diarrheal treatment hasn't been exhausted; 12. History of a second primary cancer with the exception of 1) curatively treated nonmelanomatous skin cancer, 2) curatively treated cervical or breast carcinoma in situ, or 3) other malignancy with no known active disease present and no treatment administered during the last 2 years; 13. Receipt of investigational treatment within 3 weeks of scheduled Cycle 1 Day 1 (C1D1) dosing; 14. Receipt of cytotoxic chemotherapy within 3 weeks (6 weeks for nitrosoureas and mitomycin C) of scheduled C1D1 dosing; 15. Receipt of radiation therapy within 3 weeks of scheduled C1D1 dosing, unless the radiation comprised a limited field to non-visceral structures (e.g., limb bone metastasis); 16. Receipt of treatment with immunotherapy, biological therapies, targeted small molecules, hormonal therapies within 3 weeks of scheduled C1D1 dosing; 17. Receipt of trastuzumab or ado-trastuzumab emtansine or any other experimental drug that engages the same epitope as trastuzumab within 4 weeks of scheduled C1D1 dosing; 18. Concurrent enrollment in another therapeutic clinical trial; 19. Major surgery within 3 weeks of scheduled C1D1 dosing.

### B. Study Procedures

Subjects with unknown HER2 status were consented separately in a pre-screening visit in order to undergo HER2 testing prior to screening. All subjects were screened within 28 days prior to administration of the drug (Day −28 to −1) to confirm that they meet the study selection criteria and evaluated for baseline (Day 1 predose).

In Schedule 1, subjects received the first dose of PRS-343 on Day 1 of Cycle 1 followed by subsequent doses on Day 1 of each cycle (every 3 weeks). Patient assessments occurred on Days 1, 2, 3, 4, 8, and 15 of Cycle 1; Days 1 and 2 through 8 of Cycle 2; Days 1, 2, 3, 4, 8, and 15 of Cycle 3; then on Day 1 of all subsequent cycles. Assessments also occurred on Day 21 (±7 days) of Cycles 2, 4, 6, and 8 and Day 21 of every 4 cycles (12 weeks [±7 days]) thereafter.

In Schedule 2, subjects received the first dose of PRS-343 on Day 1 of Cycle 1 followed by a dose on Day 15 of Cycle 1 and subsequent doses on Days 1 and 15 of each cycle (every 4 weeks). Patient assessments occurred on Days 1, 2, 3, 4, 8, 15, and 22 of Cycle 1; Days 1, 2 through 8, and 15 of Cycle 2; Days 1, 2, 3, 4, 8, and 15 of Cycle 3; then on Days 1 and 15 of all subsequent cycles. Assessments also occurred on Day 28 (±7 days) of Cycles 2, 4, and 6 and Day 28 of every 3 cycles (12 weeks [±7 days]) thereafter.

In Schedule 3, subjects received the first dose of PRS-343 on Day 1 of Cycle 1 followed by subsequent doses on Day 1 of each cycle (every 4 weeks). Patient assessments occurred on Days 1, 2, 3, 4, 8, and 15 of Cycle 1; Days 1 and 2 through 8 of Cycle 2; Day 1 of Cycle 3; Days 1, 2, 3, 4, 8, and 15 of Cycle 4; then on Day 1 of all subsequent cycles. Assessments also occurred on Day 28 (±7 days) of Cycles 2, 4, and 6 and Day 28 of every 3 cycles (12 weeks [±7 days]) thereafter.

Dose-limiting toxicities (DLTs) were reported during the first cycle of each schedule (e.g., 21 days after the first dose in Cycle 1 for Schedule 1). Subjects were monitored for safety throughout the study. Dosing would continue until criteria for study drug discontinuation were met (disease progression or withdrawal from the study). The subjects would return for safety follow-up on Day 30 (±3 days) after they received the last dose.

### C. Endpoints and Assessments

The primary endpoint of this study is incidence and severity of adverse effects (AEs) graded according to the National Cancer Institute Common Terminology Criteria for Adverse Events (NCI CTCAE) version 4.03. The safety and tolerability of PRS-343 was also assessed based on vital signs, physical examinations, ECOG performance status, electrocardiogram (ECG), and laboratory safety tests on an ongoing basis during the study.

Patients were monitored for AEs during study participation (beginning at the time study drug is first administered) and until 30 days after the last dose of study drug. Any ongoing serious adverse events (SAEs) were followed until resolution or stabilization. Assessments of vital signs included body temperature, systolic and diastolic blood pressure readings (mm Hg), pulse (beats per minute [BPM]), and respiratory rate (breaths rate per minute [BRPM]). Triplicate 12-lead ECG measurements were performed at pre-determined time points and collected within 10 minutes of the scheduled collection time, prior to the blood collection if collected at the same time. The mean of the triplicate ECG measurements performed pre-dose on Day 1 served as the patient's baseline corrected QT (QTc) value for all post-dose comparisons. Blood and urine samples were collected for laboratory assessments, including hematology, coagulation, serum chemistry, urinalysis, pregnancy screen, left-ventricular ejection fraction, cytokines, and washout blood sample.

For the primary endpoint, all subjects who received at least 1 dose of PRS-343 were included in the safety analyses. Safety data are presented in tabular and/or graphical format and summarized descriptively by dose cohort and time as appropriate. Absolute value data and changes from baseline data are summarized as appropriate.

The secondary endpoints of this study are serum PK parameters; PK and safety profile for Schedule 1, as well as Schedule 2 and Schedule 3, if applicable; tumor responses; duration of response; disease control rate; presence and/or concentration of anti-PRS-343 antibodies (ADAs); and PD markers.

Venous blood samples for the PK analysis and ADA assessment were collected at pre-determined time points. PK profiles to assess PK properties of single agent PRS-343 were collected from all enrolled subjects. The PK parameters determined for PRS-343 include, but are not limited to, the area under the curve (AUC), AUC_{24 h}, AUC_{inf}, Cₘₐₓ, time to maximum dose concentration (tₘₐₓ), and terminal half-life (t_{1/2}) of PRS-343. Tumor assessments, including tumor markers, will be performed at pre-determined time points, and tumor response and progression were assessed according to RECIST, Version 1.1. PD marker were assessed by quantifying lymphocyte subtypes or markers in tumor biopsies or peripheral blood and cytokine levels in plasma at pre-determined time points, prior, during, and after the duration of the dosing. The PD markers measured as available and feasible include, but are not limited to, IHC cell subsets (e.g., CD8, CD4, PDL-1, Ki67) assessed in pre-treatment (prior to Cycle 1, Day 1 dosing) and on-treatment tumor biopsies (Cycle 2, within Days 2-8), 4-1BB, soluble HER2, and IFN-γ assessed in pre-treatment (prior to Cycle 1, Day 1 dosing) and on-treatment plasma samples, CD8 T cells, CD4 T cells assessed in pre-treatment (prior to Cycle 1, Day 1 dosing) and on-treatment blood samples, and IHC cell subsets (e.g., CD8, CD4, PDL-1, Ki67) assessed in post-relapse (optional) tumor biopsies. Additionally, the PK/PD relationship and relationship to tumor response are explored.

### Example 4. Dose Escalation Study of PRS-343 in Patients with HER2+ Advanced or Metastatic Solid Tumors

This example provides information on this study for Cohorts 1-13 and interim data for Cohorts 1-11.

### A. Study Objectives and Overview

The study objectives were as described in Example 3.

Patients were allocated to different dose levels in dedicated cohorts including additional Cohorts 12 and 13 (Table 2) and received PRS-343 administered by intravenous (IV) infusion over 2 hours, every 3 weeks (Q3W) (Schedule 1) initially. If safety, PK, and PD data suggested a different dosing schedule should be evaluated, Schedule 2 (every 2 weeks, Q2W) or Schedule 3 (every week, Q1W) might be conducted. Separate MTDs may be determined for each schedule evaluated. Separate MTDs might be determined for each schedule evaluated.

A 1+3 dose escalation design was utilized for Cohorts 1 through 4 (0.0005 mg/kg to 0.015 mg/kg, respectively), and a 3+3 design was used for Cohorts 5 through 11 (0.05 mg/kg to 8 mg/kg, respectively). At the Cohort 11 (8 mg/kg) and above until Cohort 13 (18 mg/kg), the three dose schedules—Q1W, Q2W, and Q3W—were studied (FIG. 4).

TABLE 2
PRS-343 dose levels

| Cohort | Dose (mg/kg) |
|---|---|
| 1 | 0.0005 |
| 2 | 0.0015 |
| 3 | 0.005 |
| 4 | 0.015 |
| 5 | 0.05 |
| 6 | 0.15 |
| 7 | 0.5 |
| 8 | 1 |
| 9 | 2.5 |
| 10 | 5.0 |
| 11 | 8 |
| 12 | 12 |
| 13 | 18 |

### B. Study Procedures

The study procedures were as described in Example 3, except for that in Schedule 3, subjects received the first dose of PRS-343 on Day 1 of Cycle 1 followed by doses on Days 8 and 15 of Cycle 1 and subsequent doses on Days 1, 8, and 15 of each cycle (every 3 weeks). Patient assessments for Schedule 3 occurred on Days 1, 2, 3, 4, 8, and 15 of Cycle 1; Days 1, 2, 3, 4, 8, and 15 and Day 21 (±7 days) of Cycle 2; then on Days 1, 8, and 15 of all subsequent cycles. Assessments also occurred on Day 21 (±7 days) of Cycles 4, 6, 8, and every 3 cycles thereafter.

Particularly, patients were assessed for tumor response/progression per RECIST v1.1. For Schedule 1, patients are assessed every 6 weeks for the initial 24 weeks of dosing (first 8 cycles). After the week 24 scans, tumor assessments are conducted every 12 weeks. For Schedules 2 and 3, patients are assessed every 8 weeks for the initial 24 weeks of dosing (first 6 cycles for Schedule 2 and first 8 cycles for Schedule 3). After the week 24 scans, tumor assessments are conducted every 12 weeks.

### C. Endpoints and Assessments

The study procedures were as described in Example 3.

### D. Data Analysis/Methods

(i) PK

Preliminary pharmacokinetic (PK) results of PRS-343 are available at dose levels of 0.0005, 0.0015, 0.005, 0.015, 0.05, 1, 2.5, 5 and 8 mg/kg administered every 3 weeks (Q3W) and 8 mg/kg every 2 weeks (Q2W). PRS-343 was administered as a 2-hour intravenous infusion. In the Q3W dosing regimen, PRS-343 single dose and multiple dose pharmacokinetics were characterized after the first dose (Cycle 1 Day 1) and third dose (Cycle 3 Day 1), respectively. In the Q2W dosing regimen, PRS-343 single dose and multiple dose pharmacokinetics were characterized after the first dose (Cycle 1 Day 1) and fifth dose (Cycle 3 Day 1), respectively. Serum concentration data and planned times were analyzed using non-compartmental methods and preliminary PK results are presented here.

(ii) Anti-Drug Antibody Formation

Given the relatively small sample size per cohort and more data is being collected from ongoing studies, anti-drug antibody results and conclusions should be interpreted as preliminary. Immunogenicity samples collected were analyzed using a validated assay for anti-PRS-343 antibodies (ADA) and, if the sample was confirmed positive for ADA, titer value was determined. The lowest measurable titer value of the assay was 50. A patient was considered to be ADA negative, if no ADA were detected in any immunogenicity sample. If ADA were detected, depending on the maximum titer value observed, the patient was either categorized as low-titer (value below limit of quantification, values of 50 and 150) or high-titer (any value greater than 150). Titer value cutoff of 150 was used to categorize ADA-positive patients, in part, based on significant impact of titer values greater than 150 on PRS-343 pharmacokinetics.

(iii) Efficacy

Efficacy was evaluated by tumor response for patients with measurable or evaluable disease as assessed by the Investigators using RECIST version 1.1 (Appendix 1). Duration of response was calculated for patients who achieve a complete response (CR) or partial response (PR) and was defined as the time from the date of first documented response (CR or PR) to the date of documented progression or death after achieving response. Disease control rate was defined as the percentage of patients who have achieved CR, PR, or SD (stable disease) lasting at least 12 weeks.

(iv) PD—Quantification of Treatment Induced Changes of CD8 T Cells Numbers

In order to investigate whether PRS-343 is an active drug, treatment induced PD marker changes were assessed by quantifying CD8+ T cells in tumor biopsies in pre-treatment (prior to Cycle 1, Day 1 dosing) and on-treatment tumor biopsies (Cycle 2, within Days 2-8) by immunohistochemistry (IHC) staining.

Core needle biopsies were taken as specified by the clinical protocol, formaldehyde fixed and paraffin embedded, and sectioned in 3 uM sections for chromogenic IHC with anti-CD8 antibodies as well as other markers. Pathology guided digital annotations of tumor cells and stroma areas were performed. CD8+ T cells were counted per mm² of tumor cells, tumor stroma, and full tumor tissue (tumor stroma+ tumor cells).

### E. Preliminary Results

A total of 52 patients have been treated with PRS-343 administered as a single agent (Tables 3 and 4). The median age at treatment is 61 years and 32 (62%) of the patients were female. Forty (77%) of the treated patients had ECOG PS of 1 and the rest had a PS of 0. This was a previously heavily treated population of patients with 20 or 38% having received 5+ lines of therapy, 10 (19%) having received 4 lines of therapy and 11 or 21% having received 3 lines of therapy. Of the wide range of tumor types studied 19 (37%) had gastroesophageal cancer, 13 (25%) had breast cancer and 6 (12%) had gynecological cancer.

TABLE 3
Current enrolment of PRS-343 study

| Cohort | Dose & regimen | No. Patients |
|---|---|---|
| 1 | 0.0005 mg/kg Q3W | 1 |
| 2 | 0.0015 mg/kg Q3W | 1 |
| 3 | 0.005 mg/kg Q3W | 1 |
| 4 | 0.015 mg/kg Q3W | 2 |
| 5 | 0.05 mg/kg Q3W | 2 |
| 6 | 0.15 mg/kg Q3W | 5 |
| 7 | 0.5 mg/kg Q3W | 7 |
| 8 | 1 mg/kg Q3W | 6 |
| 9 | 2.5 mg/kg Q3W | 6 |
| 10 | 5 mg/kg Q3W | 9 |
| 11 | 8 mg/kg Q3W | 6 |
| 11B | 8 mg/kg Q2W | 6 |
| TBD (data driven) | 8 mg/kg Q2W | |
| Total | | 52 |

TABLE 4
Baseline characteristics of enrolled subjects

| | n (%) |
|---|---|
| Characteristic | |
| Age, Median (range) | 61 (29-92) |
| Gender | |
| F | 32 (62%) |
| M | 20 (38%) |
| ECOG PS | |
| 0 | 12 (23%) |
| 1 | 40 (77%) |
| Prior Therapy Lines | |
| 1 | 6 (12%) |
| 2 | 5 (10%) |
| 3 | 11 (21%) |
| 4 | 10 (19%) |
| 5+ | 20 (38%) |
| Average HER2 Targeting Treatments | |
| Breast | |
| Gastric | |
| Primary Cancer Type | |
| Biliary | 2 (4%) |
| Bladder | 2 (4%) |
| Breast | 13 (25%) |
| Colorectal | 5 (10%) |
| Gall Bladder | 2 (4%) |
| Gastroesophageal | 19 (37%) |
| Gynecological | 6 (12%) |
| Pancreatic | 1 (2%) |
| Other - Salivary Duct | 1 (2%) |
| Other - Melanoma | 1 (2%) |

Of the treatment related adverse events reported, the most common were infusion related reactions (10 incidents or 9% of all TRAE), fatigue (10 incidents or 9% of all TRAEs) and chills in 7 or 6% of all reported TRAEs (Table 5).

TABLE 5
Treatment-related adverse

| Occurred in ≥1 Patient | N = 111 \| n (%) | % Grade 3 |
|---|---|---|
| Infusion Related Reaction | 10 (9%) | 2 (2%) |
| Fatigue | 10 (9%) | 1 (1%) |
| Chills | 7 (6%) | 0 |
| Flushing | 7 (6%) | 3 (3%) |
| Nausea | 7 (6%) | 0 |
| Diarrhea | 7 (6%) | 0 |
| Vomiting | 6 (5%) | 0 |
| Non-Cardiac Chest Pain | 5 (6%) | 1 (1%) |

(i) Preliminary PK Results

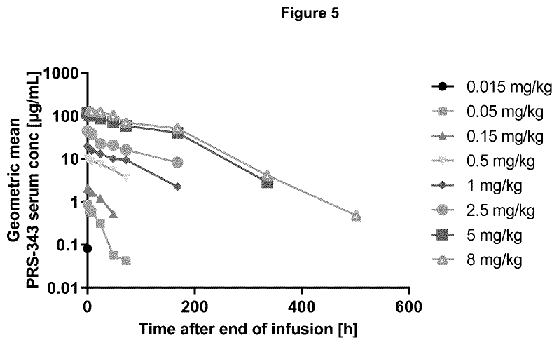

Single dose geometric mean serum concentrations are shown in FIG. 5 and preliminary PK parameters are shown in Table 6.

TABLE 6
Preliminary geometric mean (% CV) single dose (Cycle 1) PRS-343 pharmacokinetic parameters

| | | | Number of | Cₘₐₓ | tₘₐₓ | AUC₂₄ | AUC_{INF} | t_{1/2} |
|---|---|---|---|---|---|---|---|---|
| Cohort | Dose & regimen | | Patients | (μg/mL) | (h)¹ | (μg × h/mL) | (μg × h/mL) | (h) |
| 1 | 0.0005 | mg/kg Q3W | N = 1 | BLQ | | | | |
| 2 | 0.0015 | mg/kg Q3W | N = 1 | BLQ | | | | |
| 3 | 0.005 | mg/kg Q3W | N = 1 | BLQ | | | | |
| 4 | 0.015 | mg/kg Q3W | N = 2 | 0.08 | 0.08 | not | not | not |
| | | | | (56%) | (0.08-0.08) | available | available | available |
| 5 | 0.05 | mg/kg Q3W | N = 2 | 0.89 | 0.08 | 13 | 21 | 14.8 |
| | | | | (53%) | (0.08-0.08) | (21%) | (2%) | (32%) |
| 6 | 0.15 | mg/kg Q3W | N = 5 | 2.09 | 0.5 | 39 | 78 | 23.5 |
| | | | | (54%) | (0.08-8) | (53%) | (68%) | (19%) |
| 7 | 0.5 | mg/kg Q3W | N = 6 | 10.35 | 0.2 | 214 | 793 | 52.9 |
| | | | | (23%) | (0.08-8) | (28%) | (65%) | (35%) |
| 8 | 1 | mg/kg Q3W | N = 6 | 19.46 | 0.08 | 376 | 1657 | 64.3 |
| | | | | (28%) | (0.08-0.08) | (27%)² | (41%)² | (30%)² |
| 9 | 2.5 | mg/kg Q3W | N = 6 | 45.34 | 0.08 | 928 | 4530 | 74.8 |
| | | | | (35%) | (0.08-0.08) | (34%) | (77%) | (50%) |
| 10 | 5 | mg/kg Q3W | N = 7 | 119.74 | 0.1 | 2480 | 17033 | 118 |
| | | | | (15%) | (0.08-4) | (17%) | (53%) | (45%) |
| 11 | 8 | mg/kg Q3W | N = 5 | 146.39 | 0.4 | 3243 | 23930 | 104 |
| | | | | (25%) | (0.08-4) | (19%) | (39%) | (51%) |
| 11B | 8 | mg/kg Q2W | N = 6 | 142.59 | 0.2 | 3137 | 21763 | 106 |
| | | | | (31%) | (0.08-8) | (33%) | (35%) | (31%) |

BLQ below limit of quantification
¹median (range)
²n = 5

Serum PRS-343 concentration were very low or below the limit of quantitation at the 0.0005 mg/kg to 0.05 mg/kg dose levels. At the 0.15 mg/kg dose level, serum PRS-343 concentrations were measurable for 3 days postdose and at the 0.5 and 1 mg/kg dose level, serum PRS-343 concentrations were measurable up to 14 days postdose in several patients. Starting at 2.5 mg/kg dose level, serum concentrations were measurable throughout the 3-week dosing interval in several patients.

Maximum serum concentration of PRS-343 were typically observed within 5 minutes after end of infusion. In few patients, maximum serum concentrations were observed at 4 or 8 hours after the end of infusion; however, these concentrations were not substantially greater than end of infusion concentrations, except from one patient where end of infusion concentration was below limit of quantification.

In the dose range 0.5 mg/kg to 8 mg/kg, PRS-343 Cₘₐₓ and AUC₂₄ increased at a dose proportional manner. PRS-343 exhibited dose proportional AUC_{INF} at the 2.5 mg/kg to 8 mg/kg dose levels. Variability in PRS-343 pharmacokinetic parameters was low to moderate. At the 2.5 mg/kg and higher dose levels where sufficient data points were available for reliable estimation of half-life, average half-life of at least 3 days was estimated. At the highest dose of 8 mg/kg Q3W, average PRS-343 half-life was estimated to be 104 hours (4.3 days).

Cycle 3 multiple dose pharmacokinetic results are available in a limited number of patients and are discussed in the context of immunogenicity results (ADA formation).

(ii) Preliminary ADA Formation Results

Incidence of ADA in patients with at least one postdose sample analyzed for ADA is summarized in Table 7.

TABLE 7
Incidence of anti-PRS-343 antibodies (anti-drug antibodies, ADA)

| | | | Number of | Number (%) of | Number (%) of | Number (%) of | Highest titer |
|---|---|---|---|---|---|---|---|
| | | | patients with | patients with | patients with | patients with | reported |
| | | | at least 1 | no measurable | low-titer ADA | high-titer ADA | in each |
| Cohort | Dose & regimen | | postdose sample | ADA | (titer ≤ 150) | (titer > 150) | dose level |
| 1 | 0.0005 | mg/kg Q3W | 1 | 0 (0%) | 0 (0%) | 1 (100%) | 984,000 |
| 2 | 0.0015 | mg/kg Q3W | 1 | 1 (100%) | 0 (0%) | 0 (0%) | n/a |
| 3 | 0.005 | mg/kg Q3W | 1 | 1 (100%) | 0 (0%) | 0 (0%) | n/a |
| 4 | 0.015 | mg/kg Q3W | 2 | 2 (100%) | 0 (0%) | 0 (0%) | n/a |
| 5 | 0.05 | mg/kg Q3W | 1 | 0 (0%) | 0 (0%) | 1 (100%) | 12,100 |
| 6 | 0.15 | mg/kg Q3W | 5 | 4 (80%) | 0 (0%) | 1 (20%) | 1,350 |
| 7 | 0.5 | mg/kg Q3W | 6 | 1 (16.7%) | 1 (16.7%) | 4 (66.7%) | 8,860,000 |
| 8 | 1 | mg/kg Q3W | 5 | 2 (40%) | 0 (0%) | 3 (60%) | 36,500 |
| 9 | 2.5 | mg/kg Q3W | 5 | 0 (0%) | 3 (60%) | 2 (40%) | 109,000 |
| 10 | 5 | mg/kg Q3W | 5 | 3 (60%) | 1 (20%) | 1 (20%) | 450 |
| 11 | 8 | mg/kg Q3W | 5 | 2 (40%) | 2 (40%) | 1 (20%) | 36,500 |
| 11B | 8 | mg/kg Q2W | 3 | 1 (33.3%) | 1 (33.3%) | 1 (33.3) | 4,050 |

| | | | | |
|---|---|---|---|---|
| Cohorts 9, 10, 11 & 11B | 18 | 6 (33.3%) | 7 (38.9%) | 5 (27.8%) |
| All cohorts | 40 | 17 (42.5%) | 8 (20%) | 15 (37.5%) |

Out of 40 patients treated with PRS-343 at doses ranging from 0.0005 mg/kg to 8 mg/kg with at least one postdose immunogenicity sample, 17 patients were ADA negative. ADA was detected in at least one post-dose sample in the remaining 23 patients with 8 patients considered to have low titers and 15 patients considered to have high titers.

Based on overall safety profile, further evaluation of PRS-343 is expected to continue at higher dose levels. Therefore, immunogenicity data are also summarized for the three highest dose levels (currently considered to be clinically relevant) of 2.5, 5 and 8 mg/kg. In Cohorts 9 and above, out of 18 patients, 6 patients were ADA negative, 7 patients were ADA-positive with low-titer and 5 patients were ADA-positive with high-titer.

In most of ADA-positive patients, ADA was detected as early as 14 days after the first dose, the first time point of immunogenicity assessment.

Effect of ADA on pharmacokinetics of PRS-343 exposures in 11 patients with preliminary pharmacokinetic data in both Cycles 1 and 3 along with ADA titers, if applicable, are shown in Table 8.

TABLE 8
Exposure of PRS-343 in patients with both
Cycle 1 and Cycle 3 preliminary PK data

| | | Cycle 1 | Cycle 3 | |
|---|---|---|---|---|
| | PRS-343 dose | PRS-343 | PRS-343 | |
| | and dosing | AUC(0-t), | AUC(0-t), | ADA result and titer, if |
| Subject ID | regimen | μg × h/mL | μg × h/mL | applicable, up to Cycle 4 Day 1 |
| 111-001 | 0.15 mg/kg Q3W | 114 | 90.6 | C1D15: ADA negative |
| | | | | C3D1: ADA negative |
| | | | | C4D1: ADA negative |
| 111-002 | 0.15 mg/kg Q3W | 99.3 | 0.847 | C1D15: Titer 50 |
| | | | | C3D1: Titer 1350 |
| | | | | C4D1: Titer 1350 |
| 104-005 | 0.5 mg/kg Q3W | 1790 | 61.7 | C1D15: ADA negative |
| | | | | C3D1: Titer 1350 |
| | | | | C4D1: Titer 12100 |
| 106-001 | 1 mg/kg Q3W | 1320 | 200 | C1D15: Titer 12100 |
| | | | | C3D1: Titer 450 |
| | | | | C4D1: not available |
| 107-004 | 1 mg/kg Q3W | 171 | 0.592 | C1D15: not available |
| | | | | C3D1: not available |
| | | | | C3 unscheduled: Titer 4050 |
| | | | | C4D1: Titer 150 |
| | | | | C5D1: Titer 36500 |
| 108-002 | 2.5 mg/kg Q3W | 7243 | 1857 | C1D15: ADA negative |
| | | | | C3D1: ADA negative |
| | | | | C4D1: not available |
| 103-013 | 5 mg/kg Q3W | 12537 | 1968 | C1D15: Titer < 50 |
| | | | | C3D1: not available |
| | | | | C4D1: not available |
| 103-015 | 5 mg/kg Q3W | 13578 | 1516 | No data available |
| 103-009 | 8 mg/kg Q3W | 35192 | 49990 | C1D15: ADA negative |
| | | | | C3D1: ADA negative |
| | | | | C4D1: ADA negative |
| 108-005 | 8 mg/kg Q3W | 25132 | 4362 | C1D15: ADA negative |
| | | | | C3D1: ADA negative |
| | | | | C4D1: Titer 1350 |
| 103-012 | 8 mg/kg Q2W | 26353 | 217 | No data available |
| 104-006 | 8 mg/kg Q2W | 28332 | 8392 | C1D15: Titer 50 |
| | | | | C2D15: Titer 50 |
| | | | | C3D1: Titer 150 |
| | | | | C4D1: Not available |
| 107-012 | 8 mg/kg Q2W | 15227 | 1946 | No data available |
| 110-003 | 8 mg/kg Q2W ¹ | 17219 | 1330 | C1D15: ADA negative |
| | | | | C2D15: not available |
| | | | | C3D1: not available |
| | | | | C4D1: not available |

¹ Cycle 1 Day 1 PRS-343 dose: 481.6 mg; Cycle 3 Day 1 PRS-343 dose: 309 mg

Evaluation of relationship between decrease in Cycle 3 PRS-343 exposure and ADA titer values determined up to Cycle 4 Day 1 indicates that substantially lower PRS-343 exposure in Cycle 3 is associated with titer values of at least 450 with the exception of a single patient (Subject ID 104-006).

In patients without ADA, Cycle 1 and Cycle 3 exposure were comparable indicating no accumulation after Q3W administration. A patient (Subject ID 108-002) had lower exposure in Cycle 3 without ADA detected until Cycle 4 Day 1.

(iii) PK/PD Relationship

Based on the preclinical dataset demonstrating maximum activity of PRS-343 was observed in vitro at 10 nM (=2 μg/mL) and the assumption that 10% of the drug gets to the tumor, a serum concentration of ≥20 μg/mL was predicted to be needed for full activity of PRS-343 in the tumor.

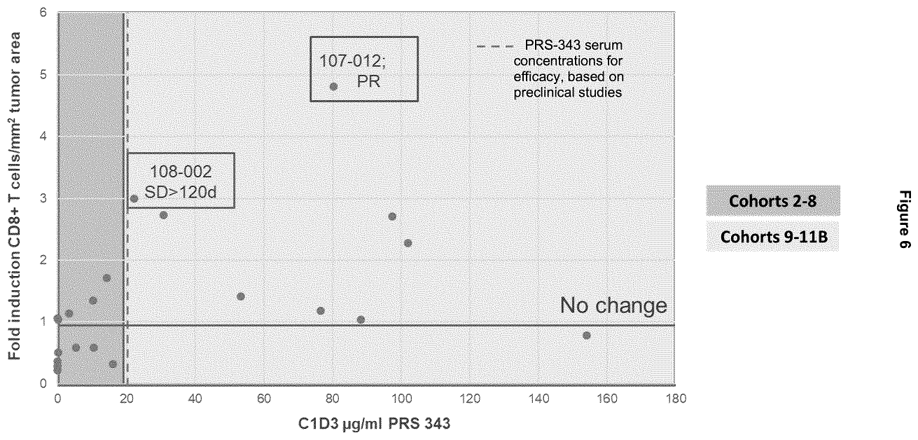

FIG. 6 shows a drug exposure/PD relationship graph. For Cohorts 1 to 8 (dose levels ranging from 0.0005 mg/kg to 1 mg/kg), the drug exposure is below 20 μg/mL. From Cohort 9 onwards (dose levels at 2.5 mg/kg and above), plasma drug levels are above 20 pg/ml.

From Cohort 9 onwards (dose levels at 2.5 mg/kg and above), strong increases in CD8+ T cell infiltration were observed in some patients, most notably for those with long lasting stable disease (SD) (108-002) and partial response (PR) (107-012) who showed a 3- and 4.8-fold induction of CD8+ T cells on treatment, respectively (FIG. 6).

These results demonstrate the 4-1BB arm activity of PRS-343 can lead to increased levels of CD8+ T cell in the tumor benefiting patients and indicate dose levels at 2.5 mg/kg and above are in the active dose range as evidenced by the strong immune-stimulatory effect of PRS-343.

(iv) Drug Activity and Emergent Determinants of Response

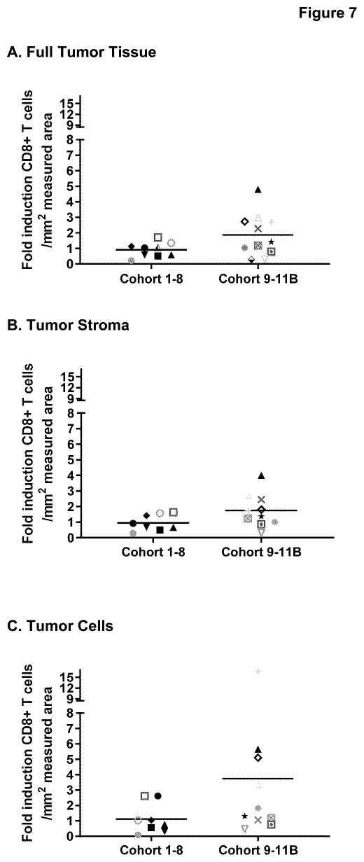

Based on the observation that more pronounced increase of CD8+ T cells is measured in patients receiving doses 2.5 mg/kg from Cohort 9 onwards (FIG. 7), PRS-343 induced increases in CD8+ T cell numbers were quantified for higher dose cohorts (Cohorts 9-11B) and compared to lower dose cohorts (Cohorts 1-8).

On average, in full tumor tissue, a 2-fold induction of CD8+ T cells in high dose cohorts as compared to low dose cohorts were observed (FIG. 7). Additionally, CD8+ T cell changes are more pronounced in the HER2+ tumor cells (FIG. 7B) as compared to the tumor stroma and full tumor tissue (FIGS. 7A and 7C) consistent with the mode of action of a HER2/4-1BB bispecific disclosed herein which forces a proximity of HER2+ tumor cells and 4-1BB-expressing CD8+ T cells.

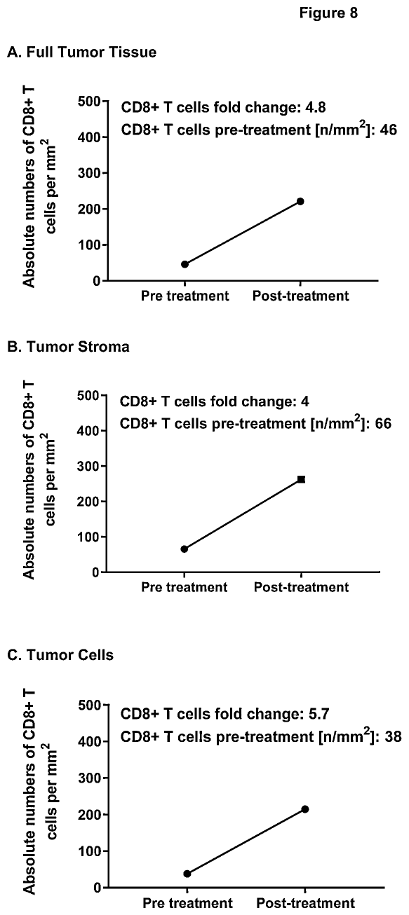

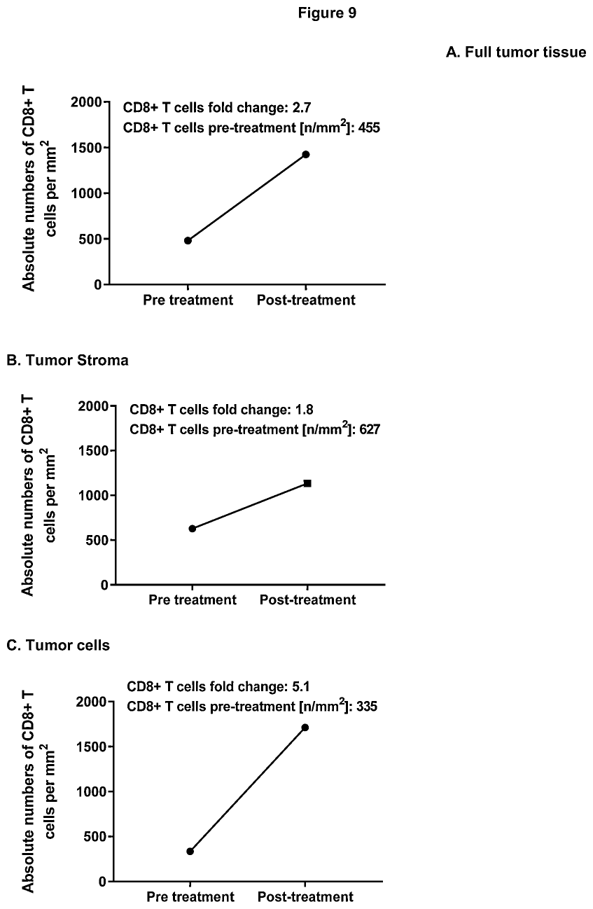

Further evidence for drug activity stems from data showing that the CD8+ T cell increases are particularly strong in patients benefiting from the treatment, e.g., patient 108-002 with SD>120d (FIGS. 7A and 9) and patient 107-012 with PR (FIGS. 7A and 8).

Exemplary results of the responding patients 107-012 and 108-002 are shown in FIG. 8 and FIG. 9, respectively. Surprisingly, patient 107-012 showed very low CD8+ T cell numbers in biopsies prior to treatment—46 CD8+ T cells/mm² of full tumor tissue, which increased on treatment by 4.6-fold. The fold increase of CD8+ T cells for both patients were more pronounced in tumor cells (5.7-fold for patient 107-012 and 5.1-fold for patient 108-002) as compared to tumor stroma (4-fold for patient 107-012 and 1.9-fold for patient 108-002), which is consistent with the mode of action of a HER2/4-1BB bispecific molecule disclosed herein, driving a proximity relationship of HER2+ tumor cells with a 4-1BB+/CD8+ T cells.

Current literature evidence suggests, depending on the indication, that check point molecules need ≥250 CD8+ T cells/mm2 in tumor tissue prior to treatment for the drugs to show efficacy in patients (Blando et al., 2019, Chen et al., 2016, Tumeh et al., 2014). Surprisingly, responding patients 107-012 and 103-012 in Cohort 11B showed very low numbers of CD8+ T cells in biopsies prior to treatment—46 and 110 CD8+ T cells/mm2 in tumor tissue, respectively. This suggests that a 4-1BB based bispecific drug as disclosed herein, can produce patient benefit where standard check point drugs cannot.

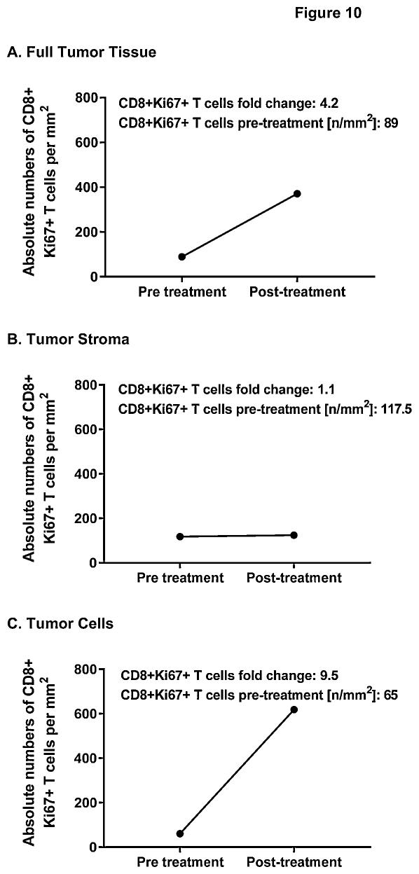

Exemplary results on CD8+Ki67+ T cell expansion of the responding patient 108-002 are also presented herein (FIG. 10). Notably, the CD8+Ki67+ T cell expansion was only observed in tumor cells (FIG. 10C) but not in tumor stroma (FIG. 10B), further suggesting a 4-1BB based bispecific drug as described herein activates CD8+ T cells only in the vicinity of tumor cells.

(v) Tumor Response

From pre-clinical data and PK/PD correlations in the study population, it was estimated that 20 μg/mL is the serum concentration of the drug which results in an efficacious dose in the tumor microenvironment. This serum concentration was reached in Cohort 9. Eighteen evaluable patients are present in Cohorts 9-11B, of which 2 patients recorded a partial response and 8 patients showed stable disease (Table 9).

TABLE 9
Summary of Response at Active Dose Range of PRS-343

| Cohort | 11B | 11 | 10 | 9 | |
|---|---|---|---|---|---|
| Best | 8 mg/kg, | 8 mg/kg, | 5 mg/kg, | 2.5 mg/kg, | |
| Response | Q2W | Q3W | Q3W | Q3W | Total |
| Response | 5 | 4 | 4 | 5 | 18 |
| Evaluable | | | | | |
| Patients | | | | | |
| CR/PR | —/2* | —/— | —/— | —/— | —/2 |
| SD | 3 | 2 | 1 | 2 | 8 |
| PD | — | 2 | 3 | 3 | 8 |
| ORR | 40% | 0% | 0% | 0% | 11% |
| DCR | 100% | 50% | 25% | 40% | 55% |

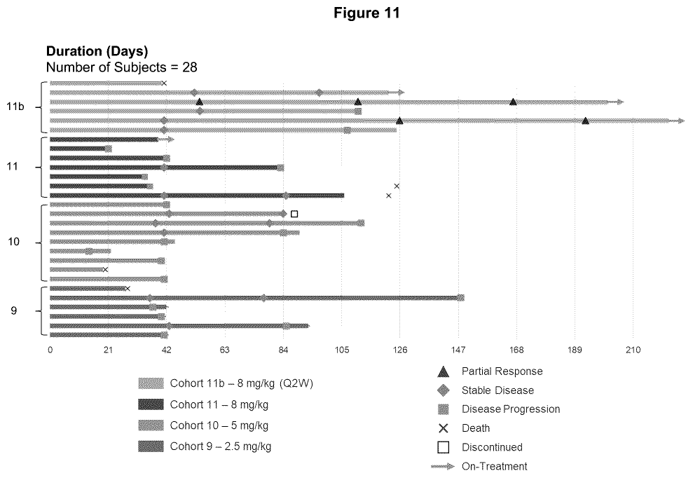

FIG. 11 depicts treatment duration of patients on PRS-343. In Cohort 9 (2.5 mg/kg, Q3W), patients stayed on study (defined as the time between Cycle 1 Day 1 to the End of Treatment visit) for an average of 69 days (standard deviation or SD of 54 days), Cohort 10 (5 mg/kg, Q3W) patients stayed on study for an average of 50 days (SD of 39 days), in Cohort 11 (8 mg/kg, Q3W) patients stayed on study for an average of 49 days (SD of 39 days), and in Cohort 11B (8 mg/kg, Q2W) patients stayed on study for an average of 119 days (SD of 9 days). The increasing length of duration on study with increasing doses may correspond to increased serum concentrations of the drug and increased probability and duration of disease response.

### Example 5. Dose Escalation Study of PRS-343 in Patients with HER2+ Advanced or Metastatic Solid Tumors

This example provides data for Cohorts 1-13 as well as the obinutuzumab (obi) pre-treatment cohort. Example 4 provides data for Cohorts 1-13, and Example 3 provides data for Cohorts 1-11.

### A. Study Objectives and Overview

The study objectives are as described in Example 3.

Patients are allocated to different dose levels in dedicated Cohorts 1 through 13 and receive PRS-343, as described in Examples 4.

The potential of obinutuzumab pre-treatment to reduce formation of ADA is studied in an up to ten patients receiving PRS-343 at a dose of 8 mg/kg per Schedule 2 (Q2W) (corresponding to Cohort 11). Further doses and schedules with B cell depletion may be tested. If obinutuzumab is shown to reduce ADA formation, and no new safety concerns arise this strategy may be used for B cell depletion and reduction of ADA incidence in further patients receiving PRS-343.

Subject inclusion criteria are as described in Example 3, so as the exclusion criteria, with the addition that: 7. Patients with latent or active hepatitis B infection are excluded from the pre-treatment cohort receiving obinutuzumab; 9. Systemic steroid therapy (>10 mg daily prednisone or equivalent) or any other form of immunosuppressive therapy within 7 days prior to the first dose of study treatment (Note: topical, inhaled, nasal and ophthalmic steroids are not prohibited). This criterion does not apply to patients receiving obinutuzumab as pre-treatment.

### B. Study Procedures

The study procedures are as described in Examples 3 and 4.

For subjects enrolled in obinutuzumab pre-treatment cohorts (receiving PRS-343 Q2W at 8 mg/kg), obinutuzumab is administered according to the GAZYVA® (obinutuzumab) package insert or institutional guidelines.

### C. Endpoints and Assessments

The study procedures are as described in Example 3. For laboratory assessments, hepatitis B virus (HBV) infection is also assessed as active and latent infection with HBV are ruled out before obinutuzumab administration.

### Example 6. Dose Escalation Study of PRS-343 in Patients with HER2+ Advanced or Metastatic Solid Tumors

This example provides information on this study for Cohorts 1-13 as well as the obinutuzumab pre-treatment cohort and provides (further) interim data for these cohorts.

### A. Study Objectives and Overview

The study objectives were as described in Examples 3, 4 and 5.

Patients were allocated to different dose levels in dedicated cohorts, as indicated in Table 10, and received PRS-343 administered by intravenous (IV) infusion over 2 hours every 3 weeks (Q3W; dosing on day 1; 21-day cycle), every 2 weeks (Q2W; dosing on days 1 and 15; 28-day cycle) and every week (Q1W; dosing on days 1, 8 and 15; 21-day cycle), respectively.

TABLE 10
Patient cohorts of PRS-343 study

| Cohort | Dose & Regimen |
|---|---|
| 1 | 0.0005 mg/kg Q3W |
| 2 | 0.0015 mg/kg Q3W |
| 3 | 0.005 mg/kg Q3W |
| 4 | 0.015 mg/kg Q3W |
| 5 | 0.05 mg/kg Q3W |
| 6 | 0.15 mg/kg Q3W |
| 7 | 0.5 mg/kg Q3W |
| 8 | 1 mg/kg Q3W |
| 9 | 2.5 mg/kg Q3W |
| 10 | 5 mg/kg Q3W |
| 11 | 8 mg/kg Q3W |
| 11B | 8 mg/kg Q2W |
| 11C | 8 mg/kg Q1W |
| 12B | 12 mg/kg Q2W |
| 13B | 18 mg/kg Q2W |
| Obi + 11B | 8 mg/kg Q2W |

Subject inclusion and exclusion criteria were as described in Example 3. Key inclusion criteria were: diagnosis of HER2+ advanced/metastatic solid tumor malignancy that has progressed on standard therapy or for which no standard therapy is available; HER2+ solid tumors documented by ASCO, CAP or institutional guidelines; patients with breast, gastric and GEJ cancer must have received at least one prior HER2-targeted therapy for advanced/metastatic disease; measurable disease per RECIST v1.1; ECOG 0 or 1; adequate liver, renal, cardiac and bone marrow function. Key exclusion criteria were: ejection fraction below the lower limit of normal with trastuzumab and/or pertuzumab; systemic steroid therapy or any other form of immunosuppressive therapy within seven days prior to registration; known, symptomatic, unstable or progressing CNS primary malignancies; radiation therapy within 21 days prior to registration (limited field radiation to non-visceral structures is allowed, e.g., limb bone metastasis.

### B. Study Procedures

The study procedures were as described in Example 4 (see also Example 5 regarding pre-treatment with obinutuzumab).

### C. Endpoints and Assessments

The study procedures were as described in Examples 3 and 5. In addition, levels of circulating s4-1BB were assessed. s4-1BB has been previously shown to be increased in the sera of patients treated with an anti-4-1BB agonistic monoclonal antibody (Segal et al., 2018).

### D. Data Analysis/Methods

Data analysis and methods were as described in Example 4.

Serum s4-1BB levels were assessed by means of a proprietary enzyme-linked immunosorbent assay (ELISA). An alternative assay for assessing serum s4-1BB levels is described in Segal et al., 2018.

The percentage of PD-L1-positive cells (IC score) was determined by immunohistochemistry (IHC) staining.

### E. Preliminary Results

A total of 74 patients have been treated with PRS-343 administered as a single agent (Tables 10 and 11). The median age at treatment is 63 years and 44 (59%) of the patients were female. 55 (74%) of the treated patients had ECOG PS of 1 and the rest had a PS of 0. This was a previously heavily treated population of patients with 28 or 38% having received 5+ lines of therapy, 11 (15%) having received 4 lines of therapy and 15 or 21% having received 3 lines of therapy. Of the wide range of tumor types studied 27 (36%) had gastroesophageal cancer, 16 (22%) had breast cancer and 10 (14%) had colorectal cancer.

TABLE 11
Baseline characteristics and primary
cancer types of enrolled subjects

| | n (%) |
|---|---|
| Characteristic | |
| Age, Median (range) | 63 (24-92) |
| Gender | |
| F | 44 (59%) |
| M | 30 (41%) |
| ECOG PS | |
| 0 | 19 (26%) |
| 1 | 55 (74%) |
| Prior Therapy Lines | |
| 1 | 9 (12%) |
| 2 | 10 (14%) |
| 3 | 15 (21%) |
| 4 | 11 (15%) |
| 5+ | 28 (38%) |
| Average HER2 Targeting Treatments | |
| Breast | 7 |
| Gastric | 3 |
| Primary Cancer Type | |
| Gastroesophageal | 27 (36%) |
| Breast | 16 (22%) |
| Colorectal | 10 (14%) |
| Gynecological | 9 (12%) |
| Biliary Tract | 7 (9%) |
| Bladder | 2 (3%) |
| Pancreatic | 1 (1%) |
| Other - Cancer of Unknown Origin | 1 (1%) |
| Other - Salivary Duct | 1 (1%) |

Of the treatment related adverse events reported, the most common were infusion related reactions (27 incidents or 19% of all TRAEs), fatigue (11 incidents or 8% of all TRAEs) and nausea in 11 or 8% of all reported TRAEs (Table 12). One TRAE was above grade 3: a grade 4 infusion related reaction in cohort 10 (5 mg/kg PRS-343, Q3W).

TABLE 12
Treatment-related adverse effects (TRAEs)

| Occurred in >1 Patient | N = 145 \| n (%) | % Grade 3 |
|---|---|---|
| Infusion Related Reaction | 27 (19%) | 3 (2%) |
| Fatigue | 11 (8%) | 1 (1%) |
| Nausea | 11 (8%) | 0 |
| Vomiting | 8 (6%) | 0 |
| Chills | 8 (6%) | 0 |
| Anemia | 2 (1%) | 1 (1%) |
| Arthalgia | 2 (1%) | 0 |
| Asthenia | 2 (1%) | 0 |
| Cough | 2 (1%) | 0 |
| Decreased appetite | 2 (1%) | 0 |
| Diarrhea | 6 (4%) | 0 |
| Dizziness | 2 (1%) | 0 |
| Dyspnoea | 3 (2%) | 0 |
| Flushing | 5 (3%) | 2 (1%) |
| Non-cardiac chest pain | 4 (3%) | 0 |
| Paraesthesia | 3 (2%) | 1 (1%) |
| Pruritis | 3 (3%) | 0 |
| Rash | 2 (1%) | 0 |

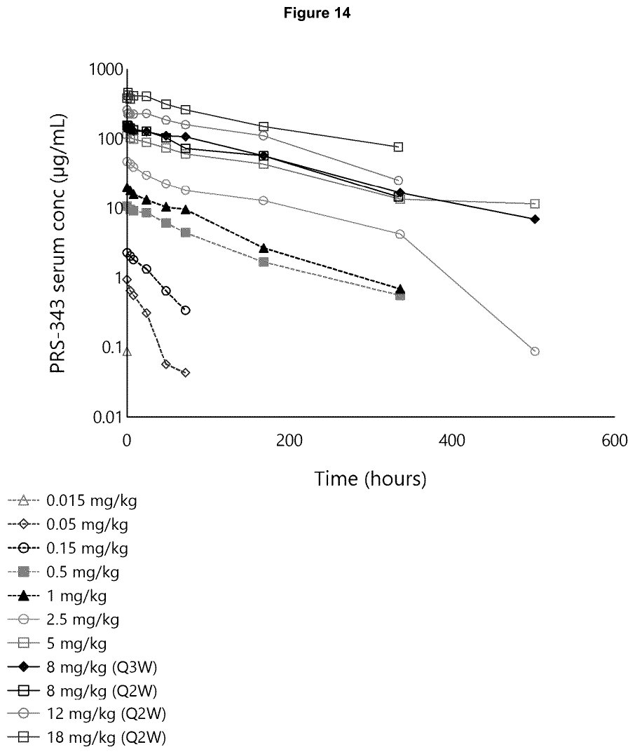

Single dose geometric mean serum concentrations of PRS-343 are shown in FIG. 14. The mean terminal half-life of PRS-343 was approximately five days. 36% of the patients were ADA-positive with titers above 1:150 in cohorts covering the active dose range (>2.5 mg/kg) (data not shown).

Based on clinical data in the study population, it was estimated that 20 μg/mL is the serum concentration of the drug which results in an efficacious dose in the tumor microenvironment. This serum concentration was reached in Cohort 9. 33 evaluable patients are present in Cohorts 9-13B, of which 1 patient recorded a complete response, 3 patients recorded a partial response and 13 patients showed stable disease (Table 13).

TABLE 13
Summary of Response at Active Dose Range of PRS-343

| Cohort | 13B | 12B | 11C | Obi | 11B | 11 | 10 | 9 | |
|---|---|---|---|---|---|---|---|---|---|
| Best | 18 mg/kg, | 12 mg/kg, | 8 mg/kg, | 8 mg/kg, | 8 mg/kg, | 8 mg/kg, | 5 mg/kg, | 2.5 mg/kg | |
| Response | Q2W | Q2W | Q1W | Q2W | Q2W | Q3W | Q3W | Q3W | Total |
| Evaluable | 3 | 2 | 4 | 2 | 7 | 4 | 6 | 5 | 33 |
| Patients | | | | | | | | | |
| CR | 1 | — | — | — | — | — | — | — | 1 |
| PR | — | — | — | — | 3 | • | — | — | 3 |
| SD | — | — | 1 | 1 | 3 | 3 | 3 | 2 | 13 |
| ORR | 33% | 0% | 0% | 0% | 43% | 0% | 0% | 0% | 12% |
| DCR | 33% | 0% | 25% | 50% | 86% | 75% | 50% | 40% | 52% |

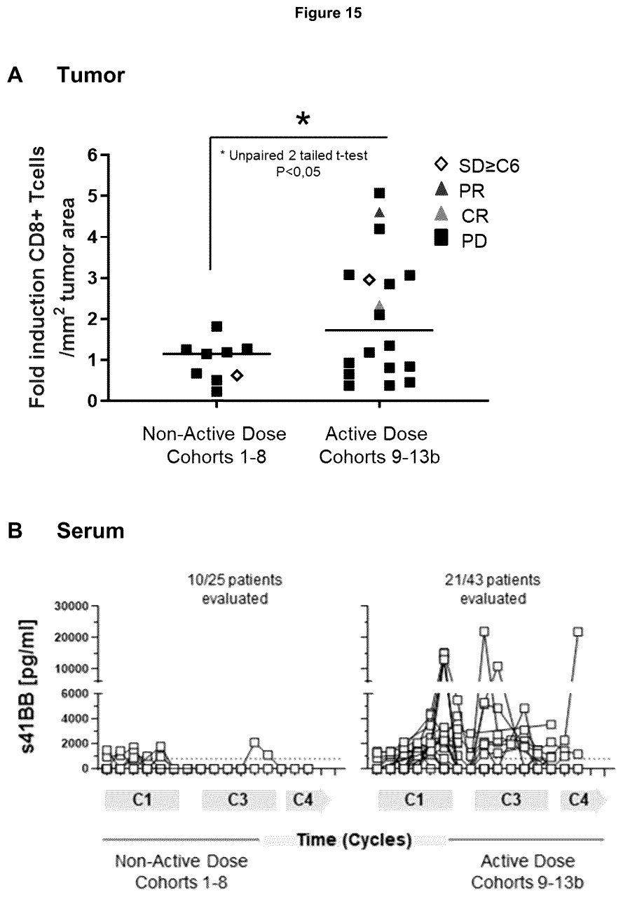

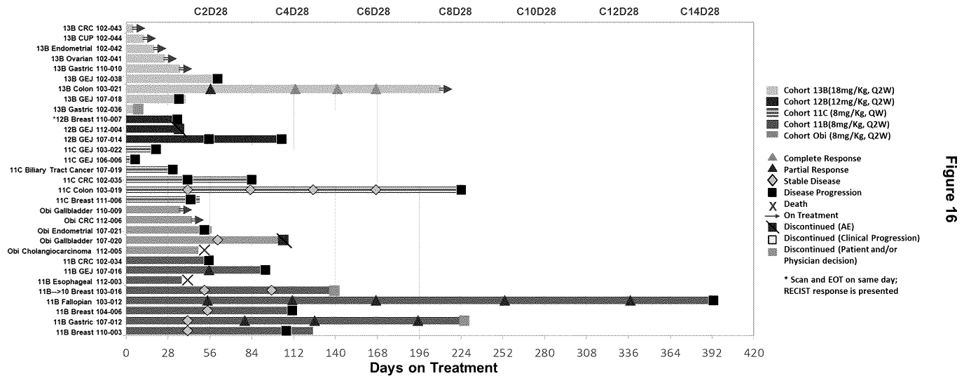

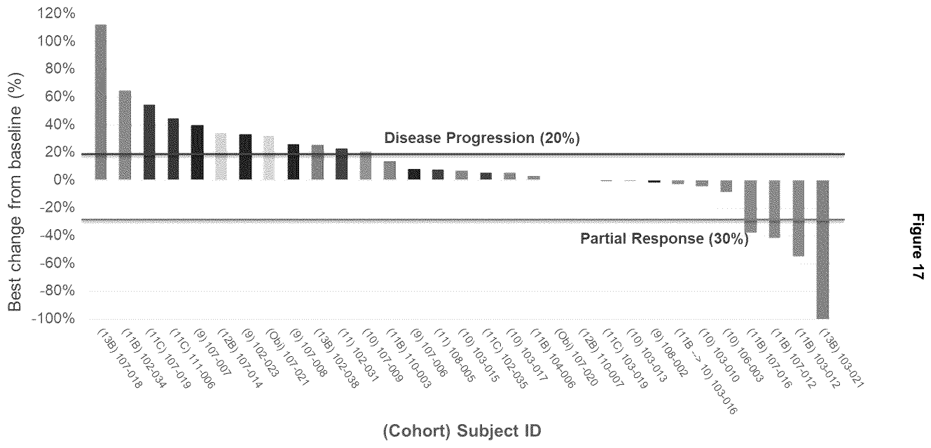

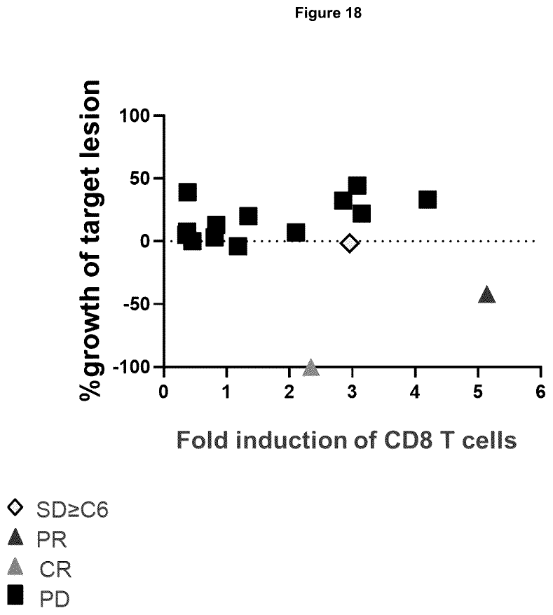

Pre-dose biopsies and post-dose biopsies (cycle 2; days 2-8) were performed. As shown in FIG. 15A, patients treated with active doses of PRS-343 (Cohorts 9-13B) showed increased CD8+ T cells in the tumor tissue. Furthermore, these patient exhibited increased levels of circulating s4-1BB in the serum (FIG. 15B), demonstrating 4-1 BB arm activity of PRS-343. The course of treatment for patients in Cohorts 11B, 11C, 12B, 13B and Obi+11B over time, including the clinical status (where applicable), such as complete response, partial response, stable disease and disease progression, is shown in FIG. 16. FIG. 17 shows the best response in target lesions for Cohorts 9, 10, 11, 11B, 11C, 12B, 13B and Obi+11B. As shown in FIG. 18, patients with prolonged clinical benefit (SD≥C6, PR and CR) exhibited an increase of CD8+ T cells in full tumor tissue.

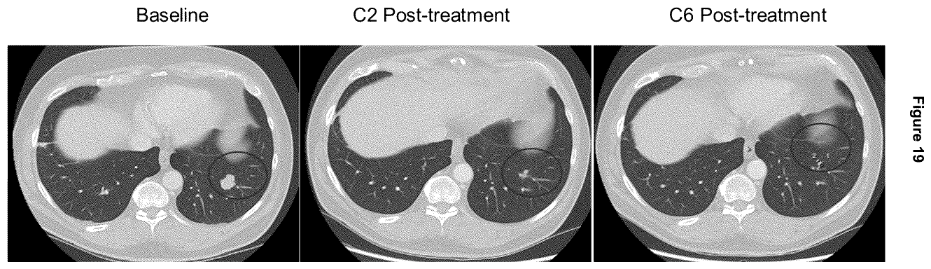

As shown in Tables 13 and 14 as well as FIGS. 16 to 19, one patient of Cohort 13B (18 mg/kg, Q2W) exhibited a complete response upon treatment with PRS-343 (see, in particular, CT scans depicted in FIG. 19). The patient is a 59-year old male with stage 4 rectal adenocarcinoma cancer which had metastasized to the heart and lung (prior therapy lines: 5+; FoundationOne HER2 amplification, in-house testing IHC3+; MSS, TMB low (2 mt/Mb)).

TABLE 14
Rectal cancer patient with confirmed CR

| | | Lesion size (mm) | | | |
|---|---|---|---|---|---|
| | | | C2 | C4 | C6 - |
| | Lesion | | Post- | Post- | Post |
| Lesions | Site | Baseline | treatment | treatment | treatment |
| Target 1 | Lung | 22 | 13 | 0 | 0 |
| % Change | — | — | −41% | −100% | −100% |
| from | | | | | |
| baseline | | | | | |
| Non-target 1 | — | Present | Present | Absent | Absent |

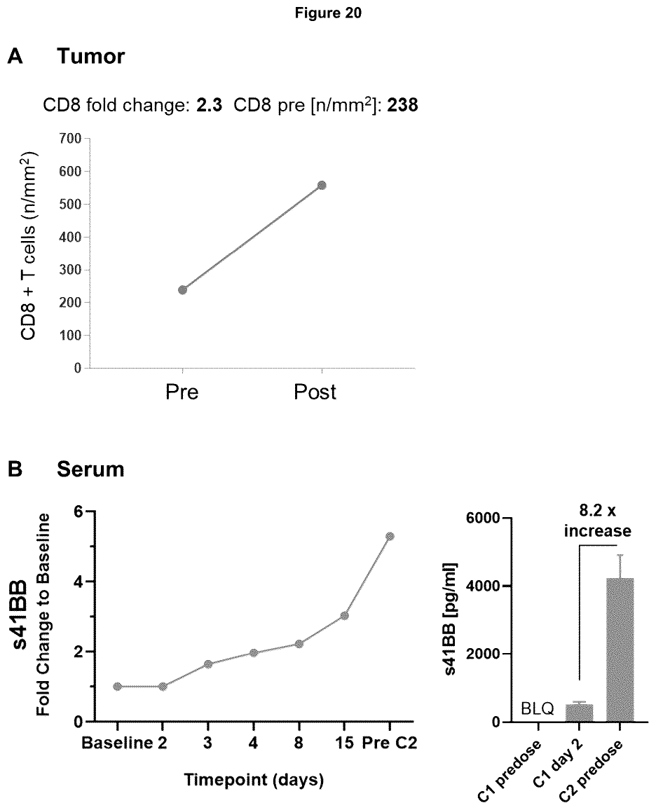

As shown in FIG. 20, post-treatment the patient exhibited increased CD8+ T cell numbers in the tumor (FIG. 20A) and increased circulating s4-1BB levels in the serum, demonstrating 4-1BB arm activity of PRS-343 (FIG. 20B).

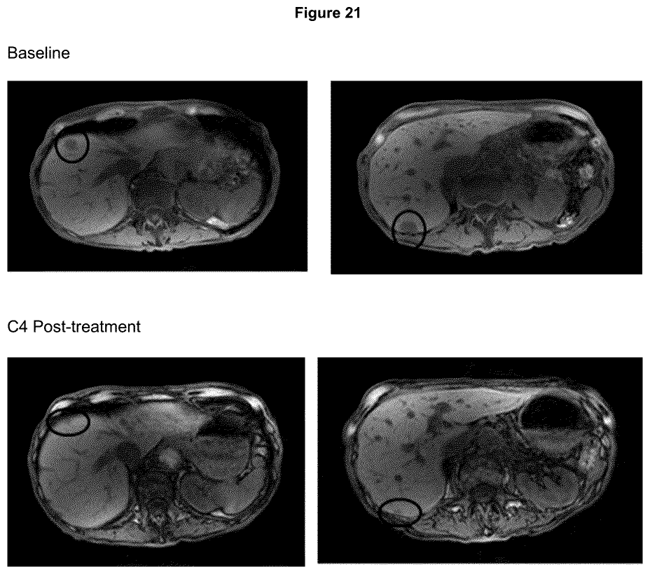

Table 15 shows the treatment outcome for a gastric cancer patient (107-012) of cohort 11B (8 mg/kg, Q2W) with confirmed partial response (see also CT scans in FIG. 21). The patient is an 80-year old woman with stage 4 gastric adenocarcinoma which had metastasized to the liver, lymph node and adrenal glands (prior therapy lines: 2; HER2 IHC3+; PD-L1-positive (CPS=3); NGS: ERBB2 amplification, TP53 mutation, alteration of CDK12 and SF3B1).

TABLE 15
Gastric cancer patient with confirmed PR

| | | Lesion Size (mm) | | | | |
|---|---|---|---|---|---|---|
| | Lesion | | C2 Post- | C3 Post- | C4 Post- | C6 Post- |
| Lesions | Site | Baseline | treatment | treatment | treatment | treatment |
| Target 1 | Liver | 14 | 12 | 10 | 9 | 8 |
| Target 2 | Liver | 20 | 16 | 10 | 8 | 9 |
| Target 3 | Pancreas | 19 | 16 | 14 | 14 | 14 |
| % Change | — | — | −17% | −36% | −42% | −42% |
| from | | | | | | |
| baseline | | | | | | |
| Non-target | Lung | Present | Present | Present | Present | Present |
| 1 | | | | | | |
| Non-target | Stomach | Present | Present | Present | Present | Absent |
| 2 | | | | | | |
| Non-target | Stomach | Present | Present | Present | Present | Absent |
| 3 | | | | | | |

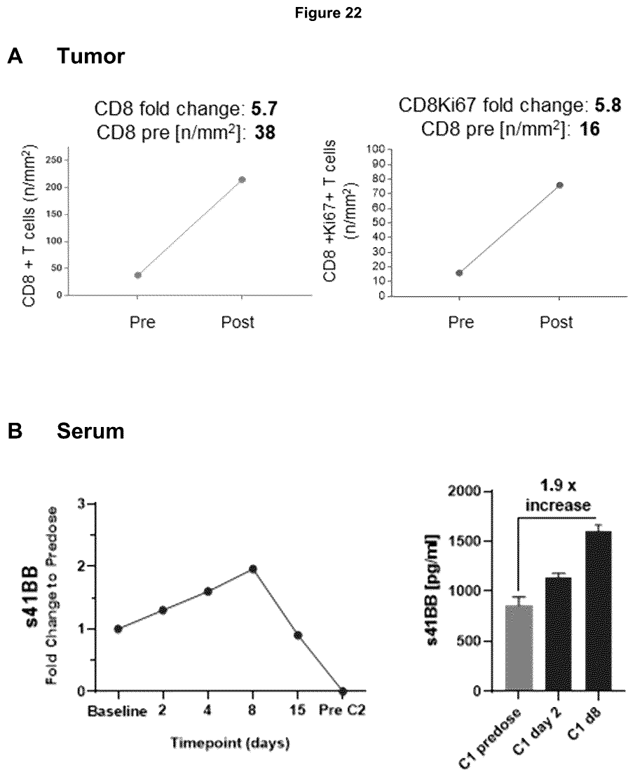

As shown in FIG. 22, post-treatment the patient exhibited increased 008+ T cell numbers and CD8+Ki67+ T cell numbers in the tumor (FIG. 22A) as well as increased circulating s4-1 BB levels in the serum, demonstrating 4-1BB arm activity of PRS-343 (FIG. 22B).

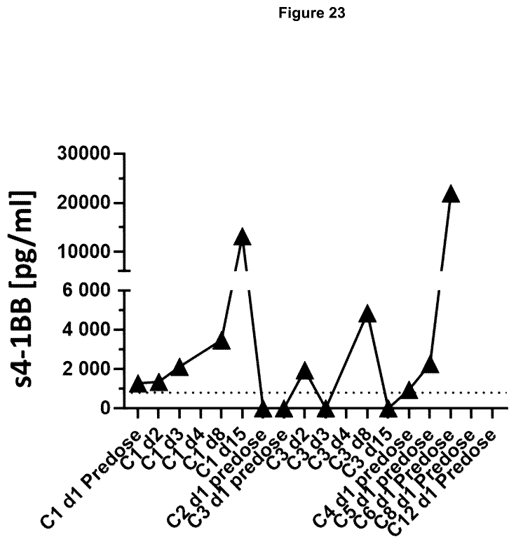

FIG. 23 shows a repeated increase of circulating s4-1BB in the serum of the PR patient 103-012 of cohort 11B (8 mg/kg, Q2W) over the course of multiple treatment cycles. The patient has fallopian tube cancer.

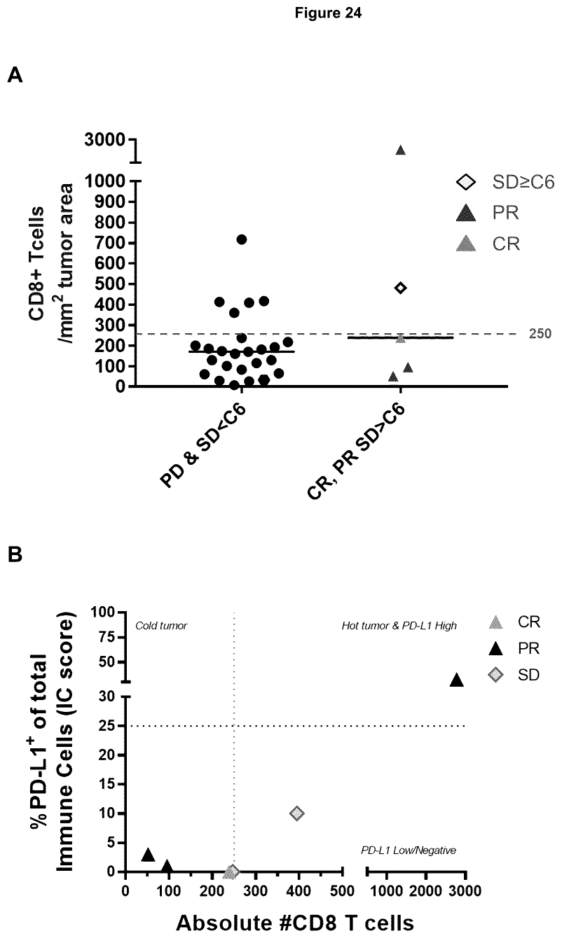

FIG. 24 shows that PRS-343 drives prolonged clinical benefit (including partial response and complete response) in patients with low C08+ T cell counts prior to therapy (<250/mm² tumor area; FIGS. 24A and B) as well as in PD-L1 low/negative patients (<25% PD-L1+ cells of total immune cells (IC score); FIG. 24B).

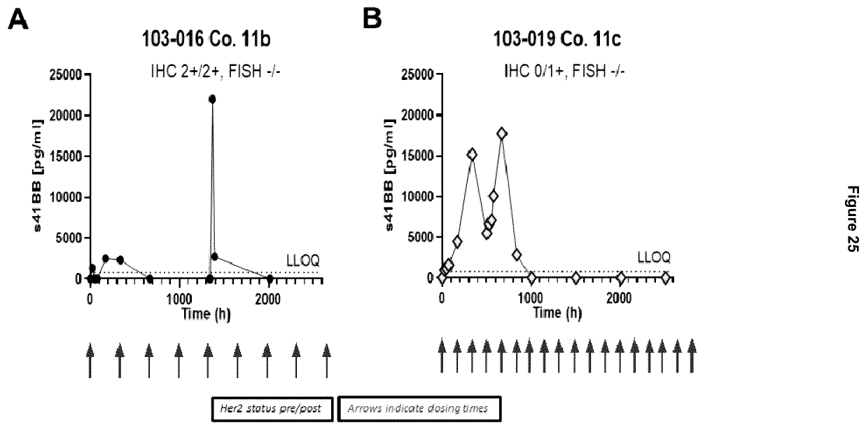

Surprisingly, additional biopsy analysis revealed that, in contrast to archival tissue assessment, the tumors of two clinically benefiting patients, breast cancer patient 103-016 (stable disease at cycles 2 and 4) and colorectal cancer patient 103-019 (stable disease at cycles 2, 4 and 6), were characterized by a low expression of HER2, as indicated by a HER2 status of IHC2+/FISH− and IHC0 or 1+/FISH−, respectively—see FIGS. 25A and B which also show the s4-1BB serum profiles of these patients, demonstrating 4-1BB arm activity of PRS-343. This indicates that PRS-343 is clinically active in the HER2 low setting, i.e., in patients which are considered as being HER2-negative based on their IHC/ISH status and which are typically not considered as being amenable to (systemic) HER2-targeting therapies.

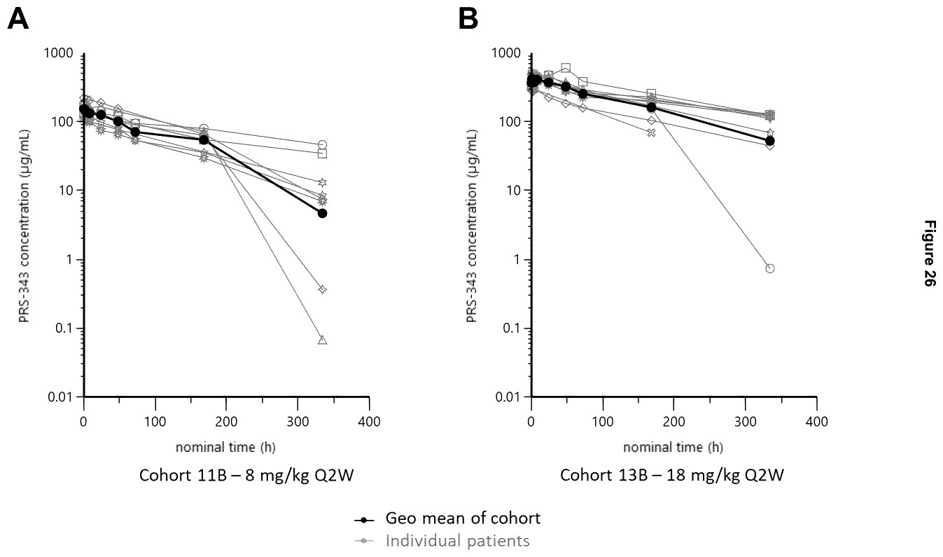

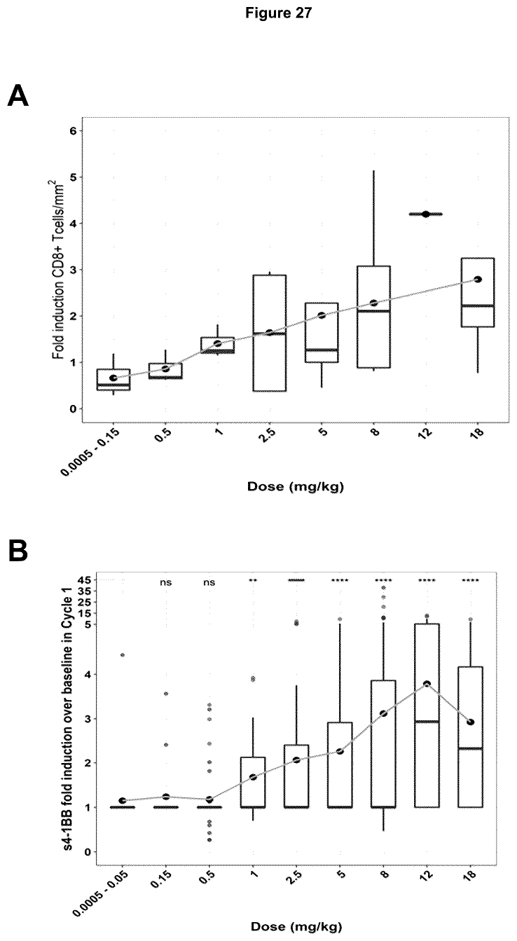

Table 16 below is an updated summary of the clinical response to PRS-343 at the active dose range, showing, inter alia, an increasing clinical benefit from PRS-343 treatment observed in cohort 13B (18 mg/ml, Q2W). Furthermore, the PRS-343 serum concentration-time profiles shown in FIGS. 14 and 26 indicate that a single dose of 18 mg/kg provides a significantly higher exposure to PRS-343 over an extended period of time than a single dose of 8 mg/kg. In addition, FIG. 27A shows dose dependency of CD8+ T cell expansion in full tumor tissue upon treatment with PRS-343, indicating a stronger PD effect with the 18 mg/kg dose. The updated clinical activity data and the PK/PD data, in additional consideration of safety aspects (e.g., with regard to the avoidance of trastuzumab-mediated adverse effects, as described, for example, in Mohan et al., 2018), provide the rationale for having a higher loading dose of, e.g., 18 mg/kg for the initial cycle to maximize PD and PK effects of PRS-343 and then a lower, but still therapeutic, dose of, e.g., 8 mg/kg, in subsequent cycles. The switch to a lower dose, e.g., 8 mg/kg, after the initial loading dose cycle is further supported by the s4-1BB profile of FIG. 27B which shows a drop of s4-1BB serum levels at the 18 mg/kg dose, indicating the potential for overactivation of the 4-1BB pathway when continuing with the higher dose after the initial cycle.

TABLE 16
Updated Summary of Response at Active Dose Range of PRS-343 (Non-Obi Cohorts)

| Cohort | 13B | 12B | 11C | 11B | 11 | 10 | 9 | |
|---|---|---|---|---|---|---|---|---|
| Best | 18 mg/kg, | 12 mg/kg, | 8 mg/kg, | 8 mg/kg, | 8 mg/kg, | 5 mg/kg, | 2.5 mg/kg, | |
| Response | Q2W | Q2W | Q1W | Q2W | Q3W | Q3W | Q3W | Total |
| Evaluable | 8 | 2 | 5 | 7 | 4 | 7 | 5 | 38 |
| Patients | | | | | | | | |
| CR | 1 | — | — | — | — | — | — | 1 |
| PR | 1 | — | — | 3 | • | — | — | 4 |
| SD | 3 | — | 1 | 3 | 3 | 3 | 2 | 15 |
| ORR | 25% | 0% | 0% | 43% | 0% | 0% | 0% | 13% |
| DCR | 63% | 0% | 20% | 86% | 75% | 43% | 40% | 53% |

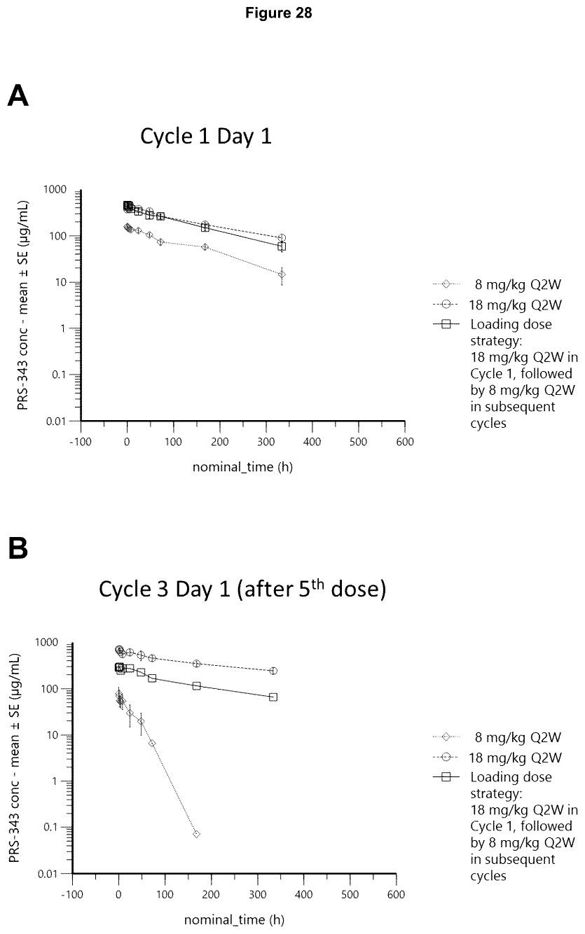

As shown in FIG. 28, additional PK analysis confirmed that a loading dose strategy (18 mg/kg for the initial cycle, 8 mg/kg in subsequent cycles) resulted in an advantageous PRS-343 serum concentration-time profile with prolonged high serum levels of PRS-343 despite the switch to a lower dose after the initial cycle.

In summary, PRS-343 showed an acceptable safety profile in all tested doses and schedules and demonstrated durable anti-tumor activity in a heavily pre-treated patient population across multiple tumor types, including those that are usually not responsive to immune therapy as well as tumors that are characterized by a low expression of HER2. Treatment with PRS-343 resulted in a clear increase in CD8+ T cell numbers and proliferative index in the tumor microenvironment of responders. Increase of soluble 4-1BB levels demonstrated activity of the 4-1BB arm of PRS-343. Based on updated clinical activity data and the totality of safety, PK and PD data, a loading dose strategy was selected, comprising the administration of a first dose, and, subsequently, of a lower second dose within the active dose range of from about 2.5 mg/kg to about 27 mg/kg (e.g., 18 mg/kg and 8 mg/kg, respectively).

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

Equivalents: Those skilled in the art will recognize or be able to ascertain using no more than routine experimentation, many equivalents to the specific embodiments of the invention described herein. Such equivalents are intended to be encompassed by the following claims. All publications, patents and patent applications mentioned in this specification are herein incorporated by reference into the specification to the same extent as if each individual publication, patent or patent application was specifically and individually indicated to be incorporated herein by reference.

### VII. NON-PATENT REFERENCES

1. ALIZADEH, A. A., GENTLES, A. J., ALENCAR, A. J., LIU, C. L., KOHRT, H. E., HOUOT, R., GOLDSTEIN, M. J., ZHAO, S., NATKUNAM, Y., ADVANI, R. H., GASCOYNE, R. D., BRIONES, J., TIBSHIRANI, R. J., MYKLEBUST, J. H., PLEVRITIS, S. K., LOSSOS, I. S. & LEVY, R. 2011. Prediction of survival in diffuse large B-cell lymphoma based on the expression of 2 genes reflecting tumor and microenvironment. Blood, 118, 1350-8.
2. ALTSCHUL, S. F., GISH, W., MILLER, W., MYERS, E. W. & LIPMAN, D. J. 1990. Basic local alignment search tool. J Mol Biol, 215, 403-10.
3. ALTSCHUL, S. F., MADDEN, T. L., SCHAFFER, A. A., ZHANG, J., ZHANG, Z., MILLER, W. & LIPMAN, D. J. 1997. Gapped BLAST and PSI-BLAST: a new generation of protein database search programs. Nucleic Acids Res, 25, 3389-402.
4. BARTLEY, A. N., WASHINGTON, M. K., VENTURA, C. B., ISMAILA, N., COLASACCO, C., BENSON III, A. B., CARRATO, A., GULLEY, M. L., JAIN, D., KAKAR, S., MACKAY, H. J., STREUTKER, C., TANG, L., TROXELL, M., AJANI, J. A. 2016. HER2 testing and clinical decision making in gastroesophageal adenocarcinoma. Arch Pathol Lab Med, 140, 1345-63.
5. BLANDO, J., SHARMA, A., HIGA, M. G., ZHAO, H., VENCE, L., YADAV, S. S., KIM, J., SEPULVEDA, A. M., SHARP, M., MAITRA, A., WARGO, J., TETZLAFF, M., BROADDUS, R., KATZ, M. H. G., VARADHACHARY, G. R., OVERMAN, M., WANG, H., YEE, C., BERNATCHEZ, C., IACOBUZIO-DONAHUE, C., BASU, S., ALLISON, J. P. & SHARMA, P. 2019. Comparison of immune infiltrates in melanoma and pancreatic cancer highlights VISTA as a potential target in pancreatic cancer. Proc Natl Acad Sci USA, 116, 1692-1697.
6. BULLIARD, Y., JOLICOEUR, R., WINDMAN, M., RUE, S. M., ETTENBERG, S., KNEE, D. A., WILSON, N. S., DRANOFF, G. & BROGDON, J. L. 2013. Activating Fc gamma receptors contribute to the antitumor activities of immunoregulatory receptor-targeting antibodies. J Exp Med, 210, 1685-93.
7. BULLIARD, Y., JOLICOEUR, R., ZHANG, J., DRANOFF, G., WILSON, N. S. & BROGDON, J. L. 2014. OX40 engagement depletes intratumoral Tregs via activating FcgammaRs, leading to antitumor efficacy. Immunol Cell Biol, 92, 475-80.
8. CHEN, P. L., ROH, W., REUBEN, A., COOPER, Z. A., SPENCER, C. N., PRIETO, P. A., MILLER, J. P., BASSETT, R. L., GOPALAKRISHNAN, V., WANI, K., DE MACEDO, M. P., AUSTIN-BRENEMAN, J. L., JIANG, H., CHANG, Q., REDDY, S. M., CHEN, W. S., TETZLAFF, M. T., BROADDUS, R. J., DAVIES, M. A., GERSHENWALD, J. E., HAYDU, L., LAZAR, A. J., PATEL, S. P., HWU, P., HWU, W. J., DIAB, A., GLITZA, I. C., WOODMAN, S. E., VENCE, L. M., WISTUBA, II, AMARIA, R. N., KWONG, L. N., PRIETO, V., DAVIS, R. E., MA, W., OVERWIJK, W. W., SHARPE, A. H., HU, J., FUTREAL, P. A., BLANDO, J., SHARMA, P., ALLISON, J. P., CHIN, L. & WARGO, J. A. 2016. Analysis of Immune Signatures in Longitudinal Tumor Samples Yields Insight into Biomarkers of Response and Mechanisms of Resistance to Immune Checkpoint Blockade. Cancer Discov, 6, 827-37.
9. CHESTER, C., SANMAMED, M. F., WANG, J. & MELERO, I. 2018. Immunotherapy targeting 4-1BB: mechanistic rationale, clinical results, and future strategies. Blood, 131, 49-57.
10. DALL'ACQUA, W. F., KIENER, P. A. & WU, H. 2006. Properties of human IgG1s engineered for enhanced binding to the neonatal Fc receptor (FcRn). J Biol Chem, 281, 23514-24.
11. DAWICKI, W. & WATTS, T. H. 2004. Expression and function of 4-1 BB during CD4 versus CD8 T cell responses in vivo. Eur J Immunol, 34, 743-751.
12. EISENHAUER, E. A., THERASSE, P., BOGAERTS, J., SCHWARTZ, L. H., SARGENT, D., FORD, R., DANCEY, J., ARBUCK, S., GWYTHER, S., MOONEY, M., RUBINSTEIN, L., SHANKAR, L., DODD, L., KAPLAN, R., LACOMBE, D. & VERWEIJ, J. 2009. New response evaluation criteria in solid tumours: revised RECIST guideline (version 1.1). Eur J Cancer, 45, 228-47.
13. FLOWER, D. R. 1996. The lipocalin protein family: structure and function. Biochem J, 318 (Pt 1), 1-14.
14. FLOWER, D. R., NORTH, A. C. & SANSOM, C. E. 2000. The lipocalin protein family: structural and sequence overview. Biochim Biophys Acta, 1482, 9-24.
15. GLAESNER, W., VICK, A. M., MILLICAN, R., ELLIS, B., TSCHANG, S. H., TIAN, Y., BOKVIST, K., BRENNER, M., KOESTER, A., PORKSEN, N., ETGEN, G. & BUMOL, T. 2010. Engineering and characterization of the long-acting glucagon-like peptide-1 analogue LY2189265, an Fc fusion protein. Diabetes Metab Res Rev, 26, 287-96.
16. HOLLIGER, P., PROSPERO, T. & WINTER, G. 1993. “Diabodies”: small bivalent and bispecific antibody fragments. Proc Natl Acad Sci USA, 90, 6444-8.
17. LEE, H. W., PARK, S. J., CHOI, B. K., KIM, H. H., NAM, K. O. & KWON, B. S. 2002. 4-1BB promotes the survival of CD8+T lymphocytes by increasing expression of Bcl-xL and Bfl-1. J Immunol, 169, 4882-8.
18. MAKKOUK, A., CHESTER, C. & KOHRT, H. E. 2016. Rationale for anti-CD137 cancer immunotherapy. Eur J Cancer, 54, 112-119.
19. MASSARELLI, E., SEGAL, N. H., RIBRAG, V., MELERO, I., GANGADHAR, T. C., URBA, W., SCHADENDORF, D., FERRIS, R. L., HOUOT, R., MORSCHHAUSER, F., LOGAN, T., LUKE, J. J., SHARFMAN, W., BARLESI, F., OTT, P. A., MANSI, L., KUMMAR, S., SALLES, G., CARPIO, C., MEIER, R., KRISHNAN, S., MCDONALD, D., MAURER, M., GU, X., NEELY, J., SURYAWANSHI, S., LEVY, R. & KHUSHALANI, N. Clinical safety and efficacy assessment of the CD137 agonist urelumab alone and in combination with nivolumab in patients with hematologic and solid tumor malignancies. 31st Annual Meeting and Associated Programs of the Society for Immunotherapy of Cancer (SITC 2016), 2016 National Harbor, MD, USA.
20. MOHAN, N., JIANG, J., DOKMANOVIC, M. & WU, W. J. 2018. Trastuzumab-mediated cardiotoxicity: current understanding, challenges, and frontiers. Antib Ther., 1, 13-17.
21. NEEDLEMAN, S. B. & WUNSCH, C. D. 1970. A general method applicable to the search for similarities in the amino acid sequence of two proteins. J Mol Biol, 48, 443-53.
22. PEARSON, W. R. & LIPMAN, D. J. 1988. Improved tools for biological sequence comparison. Proc Natl Acad Sci USA, 85, 2444-8.
23. SEGAL, N. H., HE, A. R., DOI, T., LEVY, R., BHATIA, S., PISHVAIAN, M. J., CESARI, R., CHEN, Y., DAVIS, C. B., HUANG, B., THALL, A. D. & GOPAL, A. K. 2018. Phase I Study of Single-Agent Utomilumab (PF-05082566), a 4-1BB/CD137 Agonist, in Patients with Advanced Cancer. Clin Cancer Res, 24, 1816-1823.
24. SEGAL, N. H., LOGAN, T. F., HODI, F. S., MCDERMOTT, D., MELERO, I., HAMID, O., SCHMIDT, H., ROBERT, C., CHIARION-SILENI, V., ASCIERTO, P. A., MAIO, M., URBA, W. J., GANGADHAR, T. C., SURYAWANSHI, S., NEELY, J., JURE-KUNKEL, M., KRISHNAN, S., KOHRT, H., SZNOL, M. & LEVY, R. 2017. Results from an Integrated Safety Analysis of Urelumab, an Agonist Anti-CD137 Monoclonal Antibody. Clin Cancer Res, 23, 1929-1936.
25. SILVA, J. P., VETTERLEIN, O., JOSE, J., PETERS, S. & KIRBY, H. 2015. The S228P mutation prevents in vivo and in vitro IgG4 Fab-arm exchange as demonstrated using a combination of novel quantitative immunoassays and physiological matrix preparation. J Biol Chem, 290, 5462-9.
26. SKERRA, A. 2000. Lipocalins as a scaffold. Biochim Biophys Acta, 1482, 337-50.
27. SMITH, T. F. & WATERMAN, M. S. 1981. Identification of common molecular subsequences. J Mol Biol, 147, 195-7.
28. SNELL, L. M., LIN, G. H., MCPHERSON, A. J., MORAES, T. J. & WATTS, T. H. 2011. T-cell intrinsic effects of GITR and 4-1 BB during viral infection and cancer immunotherapy. Immunol Rev, 244, 197-217.
29. TOLCHER, A. W., SZNOL, M., HU-LIESKOVAN, S., PAPADOPOULOS, K. P., PATNAIK, A., RASCO, D. W., DI GRAVIO, D., HUANG, B., GAMBHIRE, D., CHEN, Y., THALL, A. D., PATHAN, N., SCHMIDT, E. V. & CHOW, L. Q. M. 2017. Phase lb Study of Utomilumab (PF-05082566), a 4-1BB/CD137 Agonist, in Combination with Pembrolizumab (MK-3475) in Patients with Advanced Solid Tumors. Clin Cancer Res, 23, 5349-5357.
30. TUMEH, P. C., HARVIEW, C. L., YEARLEY, J. H., SHINTAKU, I. P., TAYLOR, E. J., ROBERT, L., CHMIELOWSKI, B., SPASIC, M., HENRY, G., CIOBANU, V., WEST, A. N., CARMONA, M., KIVORK, C., SEJA, E., CHERRY, G., GUTIERREZ, A. J., GROGAN, T. R., MATEUS, C., TOMASIC, G., GLASPY, J. A., EMERSON, R. O., ROBINS, H., PIERCE, R. H., ELASHOFF, D. A., ROBERT, C. & RIBAS, A. 2014. PD-1 blockade induces responses by inhibiting adaptive immune resistance. Nature, 515, 568-71.
31. VOGEL, C. L., COBLEIGH, M. A., TRIPATHY, D., GUTHEIL, J. C., HARRIS, L. N., FEHRENBACHER, L., SLAMON, D. J., MURPHY, M., NOVOTNY, W. F., BURCHMORE, M., SHAK, S., STEWART, S. J. & PRESS, M. 2002. Efficacy and safety of trastuzumab as a single agent in first-line treatment of HER2-overexpressing metastatic breast cancer. J Clin Oncol, 20, 719-26.
32. WARD, E. S., GUSSOW, D., GRIFFITHS, A. D., JONES, P. T. & WINTER, G. 1989. Binding activities of a repertoire of single immunoglobulin variable domains secreted from Escherichia coli. Nature, 341, 544-6.
33. WOLFF, A. C., HALE HAMMOND, E., ALLISON, K. H., HARVEY, B. E., MANGU, P. B., BARTLETT, J., BILOUS, M., ELLIS, I. O., FITZGIBBONS, P., HANNA, W., JENKINS, R. B., PRESS, M. F., SPEARS, P. A., VANCE, G. H., VIALE, G., MCSHANE, L. M., DOWSETT, M. 2018. Human epidermal growth factor receptor 2 testing in breast cancer. Arch Pathol Lab Med, 142, 1364-82.
34. YAO, S., ZHU, Y. & CHEN, L. 2013. Advances in targeting cell surface signalling molecules for immune modulation. Nat Rev Drug Discov, 12, 130-46.
35. ZALEVSKY, J., CHAMBERLAIN, A. K., HORTON, H. M., KARKI, S., LEUNG, I. W., SPROULE, T. J., LAZAR, G. A., ROOPENIAN, D. C. & DESJARLAIS, J. R. 2010. Enhanced antibody half-life improves in vivo activity. Nat Biotechnol, 28, 157-9.

1. A fusion protein for use in treating a HER2-expressing tumor in a subject, wherein the treatment comprises administering the fusion protein at a first dose and, subsequently, at a second dose, wherein the first dose exceeds the second dose, wherein the fusion protein comprises an antibody specific for HER2 fused at the C-terminus of both heavy chains to the N-terminus of a lipocalin mutein specific for 4-1BB, wherein the antibody comprises:
i. three heavy chain complementarity-determining regions (CDRs) shown in SEQ ID NO: 40, SEQ ID NO: 41, and SEQ ID NO: 42, and three light chain CDRs shown in SEQ ID NO: 43, SEQ ID NO: 44, and SEQ ID NO: 45; and
ii. a heavy chain with at least 95% sequence identity to the amino acid sequence shown in SEQ ID NO: 49, and a light chain with at least 95% sequence identity to the amino acid sequence shown in SEQ ID NO: 50; and
wherein the lipocalin mutein has at least 95% sequence identity to an amino acid sequence shown in SEQ ID NO: 22.

2. The fusion protein for the use of claim 1, wherein the fusion protein is administered at the first dose up to five times, up to four times, up to three times or up to two times.

3. The fusion protein for the use of claim 1 or 2, wherein the fusion protein is administered two times at the first dose.

4. The fusion protein for the use of any one of claims 1-3, wherein the first dose is from about 5 mg/kg to about 27 mg/kg.

5. The fusion protein for the use of any one of claims 1-4, wherein the first dose is from about 12 mg/kg to about 27 mg/kg.

6. The fusion protein for the use of any one of claims 1-5, wherein the first dose is about 18 mg/kg.

7. The fusion protein for the use of any one of claims 1-5, wherein the first dose is about 12 mg/kg.

8. The fusion protein for the use of any one of claims 1-7, wherein the second dose is from about 2.5 mg/kg to about 18 mg/kg.

9. The fusion protein for the use of any one of claims 1-8, wherein the second dose is from about 2.5 mg/kg to about 12 mg/kg.

10. The fusion protein for the use of any one of claims 1-9, wherein the second dose is about 8 mg/kg.

11. The fusion protein for the use of any one of claims 1-9, wherein the second dose is about 5 mg/kg.

12. The fusion protein for the use of any one of claims 1-9, wherein the second dose is about 2.5 mg/kg.

13. The fusion protein for the use of any one of claims 1-12, wherein the treatment comprises administering the fusion protein at an interval of about once every three weeks, about once every two weeks, or about once every week.

14. The fusion protein for the use of any one of claims 1-13, wherein the treatment comprises administering the fusion protein at an interval of about once every week.

15. The fusion protein for the use of any one of claims 1-13, wherein the treatment comprises administering the fusion protein at an interval of about once every two weeks.

16. The fusion protein for the use of any one of claims 1-13, wherein the treatment comprises administering the fusion protein at an interval of about once every three weeks.

17. The fusion protein for the use of any one of claims 1-5, 8, 9 and 13, comprising administering the fusion protein two times at a dose of about 18 mg/kg and, subsequently, at a dose of about 8 mg/kg, wherein the fusion protein is administered at an interval of about once every two weeks.

18. The fusion protein for the use of any one of claims 1-17, wherein the fusion protein has at least about 95% sequence identity to the amino acid sequences shown in SEQ ID NOs: 50 and 51.

19. The fusion protein for the use of any one of claims 1-18, wherein the fusion protein comprises the amino acid sequences shown in SEQ ID NO: 50 and 51.

20. The fusion protein for the use of any one of claims 1-19, wherein the fusion protein comprises two chains having the amino acid sequence shown in SEQ ID NO: 50 and two chains having the amino acid sequence shown in SEQ ID NO: 51.

21. A fusion protein for use in treating a HER2-expressing tumor in a subject, wherein the treatment comprises administering the fusion protein two times at a dose of about 18 mg/kg and, subsequently, at a dose of about 8 mg/kg, wherein the fusion protein is administered at an interval of about once every two weeks,
wherein the fusion protein comprises two chains having the amino acid sequence shown in SEQ ID NO: 50 and two chains having the amino acid sequence shown in SEQ ID NO: 51.

22. The fusion protein for the use of any one of claims 1-21, wherein the treatment is associated with:
a. an at least about 1.5-fold increase of CD8+ T cell numbers in the full tumor tissue;
b. an at least about 1.5-fold increase of CD8+ T cell numbers in tumor cells;
c. an at least about 1.5-fold increase of CD8+Ki67+ T cell numbers in the full tumor tissue;
d. an at least about 1.5-fold increase of CD8+Ki67+ T cell numbers in tumor cells;
e. an increase of CD8+ T cells from a pre-treatment level of less than about 500 per mm2 of a measured area, wherein the measured area is an area of the full tumor tissue, tumor stroma, or tumor cells;
f. an at least 30% decrease in the target lesion;
g. stable disease;
h. a partial response; or
i. a complete response.

23. The fusion protein for the use of any one of claims 1-22, wherein the tumor is selected from the group consisting of gastric cancer, gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer, colon cancer, pancreatic cancer, biliary tract cancer, salivary duct cancer, bladder cancer, and cancer of unknown primary.

24. The fusion protein for the use of any one of claims 1-23, wherein the subject has (i) a pre-treatment level of less than about 250 CD8+ T cells per mm2 of a measured area, wherein the measured area is an area of the full tumor tissue, tumor stroma, or tumor cells, and (ii) a pre-treatment level of less than about 25% PD-L1+ cells of total immune cells.

25. The fusion protein for the use of any one of claims 1-24, wherein the tumor is a HER2-positive (HER2+) tumor.

26. The fusion protein for the use of any one of claims 1-25, wherein the tumor is characterized by a HER2 status of IHC3+, IHC2+/(F)ISH+ or (F)ISH+, preferably IHC3+ or IHC2+/(F)ISH+.

27. The fusion protein for the use of claim 25 or 26, wherein the tumor exhibits HER2 gene amplification.

28. The fusion protein for the use of any one of claims 1-24, wherein the tumor is characterized by a low expression of HER2.

29. The fusion protein for the use of any one of claims 1-24 and 28, wherein the tumor is characterized by a HER2 status of IHC1+ or IHC2+/(F)ISH−.

30. The fusion protein for the use of claim 28 or 29, wherein the tumor does not exhibit HER2 gene amplification.

31. The fusion protein for the use of any one of claims 1-30, wherein the treatment further comprises administering a chemotherapeutic drug, an anti-angiogenic drug, or a combination of both.

32. A fusion protein for use in treating a tumor in a subject,
wherein the tumor is characterized by a low expression of HER2,
wherein the treatment comprises administering the fusion protein at a dose of from about 2.5 mg/kg to about 27 mg/kg,
wherein the fusion protein comprises an antibody specific for HER2 fused at the C-terminus of both heavy chains to the N-terminus of a lipocalin mutein specific for 4-1BB,
wherein the antibody comprises:
ii. three heavy chain complementarity-determining regions (CDRs) shown in SEQ ID NO: 40, SEQ ID NO: 41, and SEQ ID NO: 42, and three light chain CDRs shown in SEQ ID NO: 43, SEQ ID NO: 44, and SEQ ID NO: 45; and
iii. a heavy chain with at least 95% sequence identity to the amino acid sequence shown in SEQ ID NO: 49, and a light chain with at least 95% sequence identity to the amino acid sequence shown in SEQ ID NO: 50; and
wherein the lipocalin mutein has at least 95% sequence identity to an amino acid sequence shown in SEQ ID NO: 22.

33. The fusion protein for the use of claim 32, wherein the tumor is characterized by a HER2 status of IHC1+ or IHC2+/(F)ISH−.

34. The fusion protein for the use of claim 32 or 33, wherein the tumor does not exhibit HER2 gene amplification.

35. The fusion protein for the use of any one of claims 32-34, wherein the fusion protein is administered at an interval of about once every three weeks, about once every two weeks, or about once every week.

36. The fusion protein for the use of any one of claims 32-35, wherein the fusion protein is administered at a dose of about 2.5 mg/kg.

37. The fusion protein for the use of any one of claims 32-35, wherein the fusion protein is administered at a dose of about 5 mg/kg.

38. The fusion protein for the use of any one of claims 32-35, wherein the fusion protein is administered at a dose of about 8 mg/kg.

39. The fusion protein for the use of any one of claims 32-35, wherein the fusion protein is administered at a dose of about 12 mg/kg.

40. The fusion protein for the use of any one of claims 32-35, wherein the fusion protein is administered at a dose of about 18 mg/kg.

41. The fusion protein for the use of any one of claims 32-40, wherein the fusion protein has at least about 95% sequence identity to the amino acid sequences shown in SEQ ID NOs: 50 and 51.

42. The fusion protein for the use of any one of claims 32-41, wherein the fusion protein comprises the amino acid sequences shown in SEQ ID NO: 50 and 51.

43. The fusion protein for the use of any one of claims 32-42, wherein the fusion protein comprises two chains having the amino acid sequence shown in SEQ ID NO: 50 and two chains having the amino acid sequence shown in SEQ ID NO: 51.

44. The fusion protein for the use of any one of claims 32-43, wherein the treatment is associated with:
a. an at least about 1.5-fold increase of CD8+ T cell numbers in the full tumor tissue;
b. an at least about 1.5-fold increase of CD8+ T cell numbers in tumor cells;
c. an at least about 1.5-fold increase of CD8+Ki67+ T cell numbers in the full tumor tissue;
d. an at least about 1.5-fold increase of CD8+Ki67+ T cell numbers in tumor cells;
e. an increase of CD8+ T cells from a pre-treatment level of less than about 500 per mm2 of a measured area, wherein the measured area is an area of the full tumor tissue, tumor stroma, or tumor cells;
f. an at least 30% decrease in the target lesion;
g. stable disease;
h. a partial response; or
i. a complete response.

45. The fusion protein for the use of any one of claims 32-44, wherein the tumor is selected from the group consisting of gastric cancer, gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer, colon cancer, pancreatic cancer, biliary tract cancer, salivary duct cancer, bladder cancer, and cancer of unknown primary.
